(19) United States
(12) Patent Application Publication
(10) Pub. No.: US 2025/0041401 A1
(43) Pub. Date: Feb. 6, 2025

(54) METHODS OF IDENTIFYING HIV PATIENTS SENSITIVE TO THERAPY WITH GP120 V3 GLYCAN-DIRECTED ANTIBODIES

(71) Applicant: Gilead Sciences, Inc., Foster City, CA (US)

(72) Inventors: Brian Moldt, Overijse (BE); Craig S. Pace, Pacifica, CA (US)

(21) Appl. No.: 18/775,289

(22) Filed: Jul. 17, 2024

Related U.S. Application Data
(63) Continuation of application No. 16/877,292, filed on May 18, 2020, now Pat. No. 12,102,677.
(60) Provisional application No. 62/850,994, filed on May 21, 2019.

(51) Int. Cl. A61K 39/21; A61K 31/4745; A61K 31/519; A61P 31/18; C07K 16/10; C12Q 1/70

(52) U.S. Cl. CPC A61K 39/21; A61K 31/4745; A61K 31/519; A61P 31/18; C07K 16/1063; C12Q 1/703; C07K 2317/34; C07K 2317/76; C07K 2317/92; C12Q 2600/106; C12Q 2600/156

(57) ABSTRACT

Provided are methods for identifying patient populations infected with HIV that can be targeted by antibodies that bind to HIV gp120 V3 glycan region.

### CROSS-REFERENCE TO RELATED APPLICATIONS

This application is a continuation of U.S. nonprovisional application Ser. No. 16/877,292, filed on May 18, 2020 and granted as U.S. Pat. No. 12,102,677 on Oct. 1, 2024, which claims the benefit under 35 U.S.C. § 119 (e) of U.S. provisional application No. 62/850,994, filed on May 21, 2019, which are hereby incorporated herein by reference in their entireties for all purposes.

### SEQUENCE LISTING

The instant application contains a Sequence Listing which has been submitted electronically in XML format and is hereby incorporated by reference in its entirety. Said XML copy, created on Aug. 22, 2024, is named 1289-US-CNT_SL.xml and is 558,864 bytes in size.

### BACKGROUND

Human immunodeficiency virus (HIV) infection and related diseases are a major public health problem worldwide. Most currently approved therapies for HIV infection target the viral reverse transcriptase, protease enzymes, and integrase but resistance of HIV to these existing drugs, long term toxicity, and lack of patient adherence to daily dosing regimens have proven to be problems associated with these therapies. Therefore, it is important to discover and develop new HIV drugs.

WO 2009/066702, WO 2012/030904, WO 2014/063059, WO 2016/149698, WO 2017/106346; WO 2018/075564 and WO 2018/125813, McCoy, Retrovirology (2018) 15:70; Sok and Burton, Nat Immunol. 2018 19 (11): 1179-1188; Possas, et al., Expert Opin Ther Pat. 2018 July; 28 (7): 551-560; and Stephenson and Barouch, Curr HIV/AIDS Rep (2016) 13:31-37 describe human anti-HIV antibodies derived from memory B cells of HIV-infected donors, which target the V3 glycan region of gp120, and are capable of inhibiting infection by HIV-1 species from a plurality of clades. The therapeutic use of the antibodies may be limited due to the need to identify patients infected with HIV-1 species that can be targeted by HIV gp120 V3 glycan region antibodies.

### SUMMARY

Provided are methods of identifying patients most likely to benefit from therapy with an antibody targeting the V3 glycan region of HIV gp120.

Accordingly, in one aspect, provided are methods treating or preventing HIV in a human subject in need thereof, the method comprising: a) Identifying a human subject who is infected with an HIV or a population of HIV expressing a gp 120 comprising the following amino acid residues: a glycosylated asparagine at the position corresponding to amino acid residue position 332 (N332glycan), an aspartate at the position corresponding to amino acid residue position 325 (D325), and one or more amino acid residues selected from the group consisting of: a threonine at the position corresponding to amino acid residue position 63 (T63), a leucine at the position corresponding to amino acid residue position 179 (L179), a threonine at the position corresponding to amino acid residue position 320 (T320), and a histidine at the position corresponding to amino acid residue position 330 (H330), wherein the amino acid positions are with reference to SEQ ID NO: 4; and b) Administering to the subject an effective amount of an antibody or antigen-binding fragment thereof that competes with or comprises VH and VL regions that bind to an epitope of gp 120 within the third variable loop (V3) and/or high mannose patch comprising a N332 oligomannose glycan. In some embodiments, the method entails identifying a subject infected with an HIV or a population of HIV expressing a gp120 comprising the following amino acid residues: i. N332glycan, D325 and T63; ii. N332glycan, D325 and L179; iii. N332glycan, D325 and T320; iv. N332glycan, D325 and H330; v. N332glycan, D325, T63 and L179; vi. N332glycan, D325, T63 and T320; vii. N332glycan, D325, T63 and H330; viii. N332glycan, D325, L179 and T320; ix. N332glycan, D325, L179 and H330; x. N332glycan, D325, T320 and H330; xi. N332glycan, D325, T63, T320 and H330; xii. N332glycan, D325, T63, L179 and T320; xiii. N332glycan, D325, T63, L179 and H330; xiv. N332glycan, D325, L179, T320 and H330; or xv. N332glycan, D325, T63, L179, T320 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325 and T63; ii. N332glycan, D325 and L179; iii. N332glycan, D325 and T320; or iv. N332glycan, D325 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, T63 and L179; ii. N332glycan, D325, T63 and T320; iii. N332glycan, D325, T63 and H330; iv. N332glycan, D325, L179 and T320; v. N332glycan, D325, L179 and H330; or vi. N332glycan, D325, T320 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, L179, T320 and H330; ii. N332glycan, D325, T63, T320 and H330; iii. N332glycan, D325, T63, L179 and T320; or iv. N332glycan, D325, T63, L179 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, T63 and H330; ii. N332glycan, D325, T320 and H330; iii. N332glycan, D325, L179, T320 and H330; or iv. N332glycan, D325, T63, L179, T320 and H330, wherein the amino acid positions are with reference to SEQ ID NO: 4. In some embodiments, the subject is infected with an HIV or a population of HIV expressing a gp 120 further comprising one or more of the following amino acid residues: a glycan at amino acid residue 301 (glycan301); a lysine at amino acid residue 677 (K677); an amino acid residue other than tryptophan (e.g., A, C, D, E, F, G, H, I, K, L, M, N, P, Q, R, S, T, V or Y) at position 17 (not_W17); an amino acid residue other than arginine (e.g., A, C, D, E, F, G, H, I, K, L, M, N, P, Q, S, T, V, W or Y) at position 747 (not_R747); an insertion_321.01 (e.g., an insertion of any amino acid (e.g., A, C, D, E, F, G, H, I, K, L, M, N, P, Q, R, S, T, V, W or Y) between position G321 and K322); a glutamic acid at position 429 (E429); a glutamine at position 442 (Q442); an arginine at position 335 (R335); an isoleucine at position 165 (1165); a serine at position 393 (S393); an isoleucine at position 307 (1307); a glycan at position 295 (295 glycan); and/or an asparagine at position 300 (N300), wherein the amino acid positions are with reference to SEQ ID NO: 4. In some embodiments, at least 90%, e.g., at least 91%, at least 92%, at least 93%, at least 94%, at least 95%, at least 96%, at least 97%, at least 98%, at least 99%, or 100%, of the HIV species in the population of HIV comprise the recited amino acid residues. In some embodiments, the administered antibody or antigen-binding fragment thereof competes with or comprises VH and VL regions from an antibody selected from the group consisting of GS-9722 (clipovimab), GS-9721, PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the antibody or antigen-binding fragment thereof competes with or comprises VH and VL regions from an antibody selected from the group consisting of GS-9722, GS-9721, PGT-121, PGT-121.66, PGT-121.414, PGT-124, PGT-134, GS-2872, 10-1074, 10-1074-J, PGT-122 and PGT-123. In some embodiments, the methods comprise administering an antibody comprising an Fc region comprising one or more amino acid substitutions that extend serum half-life. In some embodiments, the methods comprise administering an antibody comprising an Fc region comprising the following amino acids at the indicated positions (EU index numbering): i. Tyrosine at position 252, threonine at position 254 and glutamic acid at position 256 (YTE); or ii. Leucine at position 428 and serine at position 434 (LS). In some embodiments, the methods comprise administering an antibody comprising an Fc region comprising the following amino acids at the indicated positions (EU index numbering): i. Aspartate at position 239 and glutamate at position 332 (DE); ii. Aspartate at position 239, glutamate at position 332 and leucine at position 330 (DEL); iii. Aspartate at position 239, glutamate at position 332, alanine at position 236 (DEA); or iv. Aspartate at position 239, glutamate at position 332, alanine at position 236 and leucine at position 330 (DEAL). In some embodiments, the methods comprise administering an antigen binding fragment. In some embodiments, the antigen binding fragment is selected from the group consisting of scFv, Fab, Fab2, Fab′, F(ab′)₂, Fv, and a diabody. In some embodiments, the antibody is one or more arms of a multi-specific antibody, e.g., a bi-specific antibody. In some embodiments, the human subject is acutely infected with HIV. In some embodiments, the antibody is administered to a human subject having an HIV infection of Fiebig stage IV or earlier, e.g., Fiebig stage III, Fiebig stage II or Fiebig stage I. In some embodiments, the antibody is administered to a human subject who has not seroconverted. In some embodiments, the human subject is recently infected with HIV, e.g., within 1, 2, 3 or 4 weeks, or prior to detection, seroconversion or manifestation of symptoms. In some embodiments, the antibody is administered to a human subject having an HIV infection of Fiebig stage V or Fiebig stage VI. In some embodiments, the human subject is chronically infected with HIV. In some embodiments, the human subject is infected with HIV clade B viruses. In some embodiments, the human subject is infected with HIV clade B viruses and the methods entail identifying a subject infected with an HIV or a population of HIV expressing a gp 120 comprising the following amino acid residues: i. N332glycan, D325, T63 and H330; ii. N332glycan, D325, L179, T320 and H330; or iii. N332glycan, D325, T63, L179, T320 and H330. In some embodiments, the human subject is infected with HIV clade A viruses. In some embodiments, the human subject is infected with HIV clade C viruses. In some embodiments, the methods further comprise administering to the subject one or more additional therapeutic agents for treating an HIV infection. In some embodiments, the subject is not receiving antiretroviral therapy (ART) or ART is discontinued prior to administration of the antibody. In some embodiments, ART is discontinued after one or more administrations of the antibody or antigen-binding fragment thereof. In some embodiments, the methods further comprise administering one or more antiretroviral therapy (ART) agents to the subject. In some embodiments, the methods further comprise administering to the subject a second antibody or antigen-binding fragment thereof that binds to an epitope or region of gp 120 selected from the group consisting of: second variable loop (V2) and/or Env trimer apex; CD4 binding site (CD4bs); gp120/gp41 interface; or silent face of gp120. In some embodiments, the second antibody or antigen-binding fragment thereof binds to an epitope or region of gp 120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the second antibody or antigen-binding fragment thereof binds to an epitope or region of gp 120 in the CD4 binding site (CD4bs) and competes with or comprises VH and VL regions from an antibody selected from the group consisting of b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N6LS (VRC-HIVMAB091-00-AB), N49-P7, NC-Cow1, IOMA, CH235 and CH235.12, N49P6, N49P7, N49P11, N49P9 and N60P25. In some embodiments, the second antibody or antigen-binding fragment thereof binds to an epitope or region of gp 120 in the gp 120/gp41 interface and competes with or comprises VH and VL regions from an antibody selected from the group consisting of PGT-151, CAP248-2B, 35022, 8ANC195, ACS202, VRC34 and VRC34.01. In some embodiments, the second antibody or antigen-binding fragment thereof binds to an epitope or region of the gp 120 silent face and competes with or comprises VH and VL regions from antibody VRC-PG05. In some embodiments, the second antibody or antigen-binding fragment thereof binds to an epitope or region of gp41 in the membrane proximal region (MPER) and competes with or comprises VH and VL regions from an antibody selected from the group consisting of 10E8, 10E8v4, 10E8-5R-100° F., 4E10, DH511.11P, 2F5, 7b2, and LN01. In some embodiments, the second antibody or antigen-binding fragment thereof binds to an epitope or region of the gp41 fusion peptide and competes with or comprises VH and VL regions from an antibody selected from the group consisting of VRC34 and ACS202. In some embodiments, the methods further comprise administering to the subject a TLR agonist. In some embodiments, the TLR agonist is a TLR2 agonist, a TLR3 agonist, a TLR7 agonist, a TLR8 agonist or a TLR9 agonist. In some embodiments, the TLR7 agonist is selected from the group consisting of vesatolimod, imiquimod, and resiquimod. In some embodiments, the methods comprise multiple administrations of the antibody or antigen-binding fragment thereof, optionally with a TLR agonist, at predetermined intervals. In some embodiments, after one or more administrations of the antibody or antigen-binding fragment thereof, the subject does not exhibit symptoms of HIV or AIDS in the absence of anti-retroviral treatment (ART) for at least 6 months, at least 1 year, at least 2 years, at least 3 years, at least 4 years, at least 5 years or longer. In some embodiments, after one or more administrations of the antibody, the subject has an HIV viral load copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

In another aspect, provided are methods of identifying a human subject infected with an HIV or a population of HIV sensitive to an antibody or antigen-binding fragment thereof that competes with or comprises VH and VL regions that bind to an epitope of gp 120 within the third variable loop (V3) and/or high mannose patch comprising a N332 oligomannose glycan. In some embodiments, the methods comprise identifying in a biological sample from the human subject a gp 120 comprising the following amino acid residues: a glycosylated asparagine at the position corresponding to amino acid residue position 332 (N332glycan), an aspartate at the position corresponding to amino acid residue position 325 (D325), and one or more amino acid residues selected from the group consisting of: a threonine at the position corresponding to amino acid residue position 63 (T63), a leucine at the position corresponding to amino acid residue position 179 (L179), a threonine at the position corresponding to amino acid residue position 320 (T320), and a histidine at the position corresponding to amino acid residue position 330 (H330), wherein the amino acid positions are with reference to SEQ ID NO: 4. In some embodiments, the method entails identifying a subject infected with an HIV or a population of HIV expressing a gp 120 comprising the following amino acid residues: i. N332glycan, D325 and T63; ii. N332glycan, D325 and L179; iii. N332glycan, D325 and T320; iv. N332glycan, D325 and H330; v. N332glycan, D325, T63 and L179; vi. N332glycan, D325, T63 and T320; vii. N332glycan, D325, T63 and H330; viii. N332glycan, D325, L179 and T320; ix. N332glycan, D325, L179 and H330; x. N332glycan, D325, T320 and H330; xi. N332glycan, D325, T63, T320 and H330; xii. N332glycan, D325, T63, L179 and T320; xiii. N332glycan, D325, T63, L179 and H330; xiv. N332glycan, D325, L179, T320 and H330; or xv. N332glycan, D325, T63, L179, T320 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325 and T63; ii. N332glycan, D325 and L179; iii. N332glycan, D325 and T320; or iv. N332glycan, D325 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, T63 and L179; ii. N332glycan, D325, T63 and T320; iii. N332glycan, D325, T63 and H330; iv. N332glycan, D325, L179 and T320; v. N332glycan, D325, L179 and H330; or vi. N332glycan, D325, T320 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, L179, T320 and H330; ii. N332glycan, D325, T63, T320 and H330; iii. N332glycan, D325, T63, L179 and T320; or iv. N332glycan, D325, T63, L179 and H330, wherein the amino acid positions are with reference to SEQ ID NO: 4. In some embodiments, the method entails identifying a subject infected with an HIV or a population of HIV expressing a gp 120 comprising the following amino acid residues: i. N332glycan, D325, T63 and H330; ii. N332glycan, D325, T320 and H330; iii. N332glycan, D325, L179, T320 and H330; or iv. N332glycan, D325, T63, L179, T320 and H330, wherein the amino acid positions are with reference to SEQ ID NO: 4. In some embodiments, the subject is infected with an HIV or a population of HIV expressing a gp 120 further comprising one or more of the following amino acid residues: a glycan at amino acid residue 301 (glycan301); a lysine at amino acid residue 677 (K677); an amino acid residue other than tryptophan (e.g., A, C, D, E, F, G, H, I, K, L, M, N, P, Q, R, S, T, V or Y) at position 17 (not_W17); an amino acid residue other than arginine (e.g., A, C, D, E, F, G, H, I, K, L, M, N, P, Q, S, T, V, W or Y) at position 747 (not_R747); an insertion_321.01 (e.g., an insertion of any amino acid (e.g., A, C, D, E, F, G, H, I, K, L, M, N, P, Q, R, S, T, V, W or Y) between position G321 and K322); a glutamic acid at position 429 (E429); a glutamine at position 442 (Q442); an arginine at position 335 (R335); an isoleucine at position 165 (1165); a serine at position 393 (S393); an isoleucine at position 307 (1307); a glycan at position 295 (295 glycan); and/or an asparagine at position 300 (N300), wherein the amino acid positions are with reference to SEQ ID NO: 4. In some embodiments, the antibody or antigen-binding fragment thereof competes with or comprises VH and VL regions from an antibody selected from the group consisting of GS-9722, GS-9721, PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the antibody or antigen-binding fragment thereof competes with or comprises VH and VL regions from an antibody selected from the group consisting of GS-9722, GS-9721, PGT-121, PGT-121.66, PGT-121.414, PGT-124, PGT-134, GS-2872, 10-1074, 10-1074-J, PGT-122 and PGT-123. In some embodiments, the human subject is acutely infected with HIV. In some embodiments, the antibody is administered to a human subject having an HIV infection of Fiebig stage IV or earlier. In some embodiments, the antibody is administered to a human subject who has not seroconverted. In some embodiments, the human subject is recently infected with HIV. In some embodiments, the antibody is administered to a human subject having an HIV infection of Fiebig stage V or Fiebig stage VI. In some embodiments, the human subject is chronically infected with HIV. In some embodiments, the human subject is infected with HIV clade B viruses. In some embodiments, the human subject is infected with HIV clade B viruses and the methods entail identifying a subject infected with an HIV or a population of HIV expressing a gp 120 comprising the following amino acid residues: i. N332glycan, D325, T63 and H330; ii. N332glycan, D325, L179, T320 and H330; or iii. N332glycan, D325, T63, L179, T320 and H330. In some embodiments, the human subject is infected with HIV clade A viruses. In some embodiments, the human subject is infected with HIV clade C viruses.

With respect to further embodiments of the methods described herein, in some embodiments, the gp 120 amino acids are identified in one or more gp 120 polypeptide sequences expressed from an HIV or a population of HIV isolated from the subject. In some embodiments, the gp 120 amino acids are identified in one or more gp 120 polynucleotide sequences encoding a gp120 polypeptide from an HIV or a population of HIV isolated from the subject. In various embodiments, the methods entail performing next generation sequencing (NGS) on polynucleotide sequences encoding gp 120 from a population of HIV. In some embodiments, the gp 120 variants are detected to a frequency level of about 1%, e.g., to a frequency level of about 0.5%, of the virus population. In some embodiments, the gp 120 amino acids are identified in one or more biological samples from the subject, wherein the one or more biological sample are obtained from blood, peripheral blood mononuclear cells (PBMCs), serum, plasma, semen or lymph nodes. In some embodiments, the methods entail identifying a population of HIV RNA in a serum or plasma sample. In some embodiments, the methods further comprise the step of obtaining one or more biological samples from the subject. In some embodiments, two or more biological samples are obtained from the subject. In some embodiments, two or more biological samples are obtained from the same tissue or fluid at two or more different time points. In some embodiments, two or more biological samples are obtained from different tissues or fluids, or from different anatomical locations.

### Definitions

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”. Where the terms “comprise” or “comprising” are used herein, it is understood that the disclosure further includes embodiments wherein these terms are replaced with “consist of” or “consist essentially of” or “consisting of” or “consisting essentially of.”

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment described herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

A “composition” can comprise an active agent, e.g., a contrast agent and a carrier, inert or active, e.g., a pharmaceutically acceptable carrier, diluent or excipient. A composition may be a pharmaceutical composition. In particular embodiments, the compositions are sterile, substantially free of endotoxins or non-toxic to recipients at the dosage or concentration employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

A “biological sample” or “sample” refers to any fluid, cellular or solid tissue sample from a subject that has or is suspected of having detectable HIV.

A “subject,” “individual” or “patient” refers to any mammal, including humans and non-human primates. In particular embodiments, the mammal is human.

The term “buffer” as used herein denotes a pharmaceutically acceptable excipient, which stabilizes the pH of a pharmaceutical preparation. Suitable buffers are well known in the art. Suitable pharmaceutically acceptable buffers include but are not limited to acetate-buffers, histidine-buffers, citrate-buffers, succinate-buffers, tris-buffers and phosphate-buffers. In certain embodiments, the concentration of the buffer is from about 0.01 mM to about 1000 mM, about 0.1 mM to about 1000 mM, about 0.1 mM to about 500 mM, about 0.1 to about 200 mM, about 0.1 to about 100 mM, about 1 mM to about 1000 mM, about 1 mM to about 500 mM, about 1 mM to about 200 mM, about 1 mM to about 100 mM, about 1 mM to about 50 mM, about 2 mM to about 60 mM, about 4 mM to about 60 mM, or about 4 mM to about 40 mM, about 5 mM to about 20 mM, or about 5 mM to about 25 mM.

“Optional” or “optionally” means that the subsequently described event of circumstances may or may not occur, and that the description includes instances where said event or circumstance occurs and instances in which it does not.

“Pharmaceutical composition” refers to a formulation of a compound and a medium generally accepted in the art for the delivery of the biologically active compound to mammals, e.g., humans. Such a medium may include any pharmaceutically acceptable carriers, diluents or excipients therefore.

“Effective amount” or “therapeutically effective amount” refers to that amount of an antibody or antigen-binding fragment thereof that, when administered alone or in combination with another therapeutic agent to a cell, tissue, or subject is sufficient to effect treatment or a beneficial result in the subject. The amount which constitutes an “effective amount” will vary depending on the antibody or antigen-binding fragment thereof and its specific use, and potentially also the condition and its severity, the manner of administration, and the age of the subject to be treated, but can be determined routinely by one of ordinary skill in the art having regard to his own knowledge and to this disclosure. A therapeutically effective dose further refers to that amount of the antibody or antigen-binding fragment thereof sufficient to treat, prevent or ameliorate an infection or disease condition or the progression of an infection or disease, and that amount sufficient to effect an increase in rate of treatment, healing, prevention or amelioration of such conditions. When applied to an individual antibody or antigen-binding fragment thereof administered alone, a therapeutically effective dose refers to that active ingredient alone. When applied to a combination, a therapeutically effective dose refers to combined amounts of the active ingredients that result in the therapeutic effect, whether administered in combination, serially or simultaneously.

“Treat,” “treating” or “treatment” as used herein covers the treatment of the disease, injury, or condition of interest, e.g., HIV-1 infection, in a subject, e.g., a mammal, such as a human, having the disease or condition of interest, and includes: (i) inhibiting progression of the disease, injury, or condition, i.e., arresting its development; (ii) reducing or relieving the disease, injury, or condition, i.e., causing regression of the disease or condition; or (iii) relieving the symptoms resulting from the disease, injury, or condition. As used herein, the terms “disease,” “disorder,” and “condition” may be used interchangeably. As used herein, “inhibition,” “treatment,” “treating,” and “ameliorating” are used interchangeably and refer to, e.g., stasis of symptoms, prolongation of survival, partial or full amelioration of symptoms, and partial or full eradication of a condition, disease or disorder.

As used herein, “prevent” or “prevention” includes (i) preventing or inhibiting the disease, injury, or condition from occurring in a subject, in particular, when such subject is predisposed to the condition but has not yet been diagnosed as having it; or (ii) reducing the likelihood that the disease, injury, or condition will occur in the subject.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity.

The term “human antibody” refers to antibodies containing sequences of human origin, except for possible non-human CDR regions, and does not imply that the full structure of an Ig molecule be present, only that the antibody has minimal immunogenic effect in a human.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8 (10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′) 2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain typically interact to define an antigen-binding site on the surface of the VH-VL dimer. Generally, the six CDRs collectively confer antigen-binding specificity to the antibody, although there are examples of antigen-binding specificity being maintained when one or more of the six CDRs are deleted or modified, e.g., by altering the amino acid sequence of the one or more CDRs, e.g., by amino acid insertion, deletion or substitution. In addition, even a single variable domain (or half of an Fv comprising only three CDRs specific for an antigen) has the ability to recognize and bind antigen, although at a lower affinity than the entire binding site. Residues other than those present in the CDRs may also be important for or play a role in antigen binding and/or specificity as shown for PGT121 and closely related somatic variants which interact with the gp120 antigen using residues in light chain framework 3 (Julien et al. Science 342:1477-83 (2013); Julien et al. PLOS Pathog. 9: c1003342 (2013)) These residues in part arise from an unusual three amino acid insertion which extends an otherwise short surface loop in PGT121 and related somatic variants (e.g. PGT122, PGT123, PGT124, PGT133, PGT134, 10-1074) that contacts both the N332 linked glycan and protein residues on HIV Env, effectively forming an additional (e.g. a fourth) complementarity determining region (CDR) loop in the PGT121 light chain between LC CDRs 2 and 3.

The term “hypervariable region” refers to the amino acid residues of an antibody that are typically responsible for antigen-binding. The hypervariable region generally comprises amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop” VCDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with AHo; Honneger, A. and Plunkthun, A. J. Mol. Biol. 309:657-670 (2001)).

The “Fab” fragment is a region on an antibody that binds to antigens. It is composed of one constant and one variable domain of each of the heavy and light chain. These domains shape the paratope—the antigen-binding site—at the amino terminal end of the monomer. The two variable domains bind the epitope on their specific antigens. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their variable or constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “scFv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen-binding.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al, Proc. Natl. Acad. Sci. USA, 90:6444-6448 (1993).

An “isolated” antibody or antigen-binding fragment thereof is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 95% by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

An antibody or antigen-binding fragment thereof that “specifically binds to” or is “specific for” a particular polypeptide or an epitope on a particular polypeptide is one that binds to that particular polypeptide or epitope on a particular polypeptide without substantially binding to any other polypeptide or polypeptide epitope. In some embodiments, the antibody of the present disclosure specifically binds to an antigen, e.g., an HIV-1 gp120 polypeptide, with dissociation constant Kd equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM, in the form of monoclonal antibody, scFv, Fab, or other form of antibody measured at a temperature of about 4° C., 25° C., 37° C., or 42° C. Affinities of antibodies can be readily determined using conventional techniques, for example, those described by Scatchard et al. (Ann. N. Y. Acad. Sci. USA 51:660 (1949), ELISA assays, biolayer interferometry (BLI) assays, and surface plasmon resonance (SPR) assays). Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

As used herein, an antibody that “internalizes” is one that is taken up by (i.e., enters) the cell upon binding to an antigen on a mammalian cell {e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

An “antibody that inhibits the growth of infected cells” or a “growth inhibitory” antibody is one that binds to and results in measurable growth inhibition of infected cells expressing or capable of expressing an HIV1 epitope bound by an antibody. Preferred growth inhibitory antibodies inhibit growth of infected cells by greater than 20%, preferably from about 20% to about 50%, and even more preferably, by greater than 50% (e.g., from about 50% to about 100%) as compared to the appropriate control, the control typically being infected cells not treated with the antibody being tested. Growth inhibition can be measured at an antibody concentration of about 0.1 to about 30 μg/ml or about 0.5 nM to about 200 nM in cell culture, where the growth inhibition is determined 1-10 days after exposure of the infected cells to the antibody. Growth inhibition of infected cells in vivo can be determined in various ways known in the art. The antibody is growth inhibitory in vivo if administration of the antibody at about 1 μg/kg to about 100 mg/kg body weight results in reduction the percent of infected cells or total number of infected cells within about 5 days to 3 months from the first administration of the antibody, preferably within about 5 to 30 days.

An antibody that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). Preferably the cell is an infected cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody that induces apoptosis is one that results in about 2- to 50-fold, preferably about 5- to 50-fold, and most preferably about 10- to 50-fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis (e.g., antibody-dependent cell-mediated phagocytosis (ADCP)); down regulation of cell surface receptors (e.g., B cell receptor); and B cell activation.

“Antibody-dependent cell-mediated cytotoxicity” or “ADCC” refers to a form of cytotoxicity in which secreted or exogenously administered Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ACC, NK cells, express FcγRIII only, whereas monocytes express FcγRI, FcγRII and FcγRIII. FcR expression on hematopoietic cells is summarized in Table 4 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the antibody or antigen-binding fragment thereof may be assessed in vivo, e.g., in an animal model such as that disclosed in Clynes et al., Proc. Natl. Acad. Sci. (USA) 95:652-656 (1998).

“Fc receptor” or “FcR” describes a receptor that binds to the Fc region of an antibody. In certain embodiments, the FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof, and FcγRIIC, which includes the FcγRIIB extracellular domain fused to an activating cytoplasmic region. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Dacron, Annu. Rev. Immunol. 15:203-234 (1997)). FcRs are reviewed in Ravetch and Kinet, Annu. Rev. Immunol 9:457-92 (1991); Capel et al, Immunomethods 4:25-34 (1994); and de Haas et al, J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al, J. Immunol. 117:587 (1976) and Kim et al, J. Immunol. 24:249 (1994)), and which plays a role in salvaging IgG from lysosomal degradation by FcRn dependent recycling following endocytosis. FcRn binding following pinocytosis in endothelial cells has been shown to be important for sustaining the prolonged pharmacokinetic half-life of antibodies. Assessment of pH dependent human FcRn binding of antibodies in vitro may be performed to provide a prediction of potential for favorable clinical pharmacokinetics (Datta-Mannan and Wroblewski, Drug Metab. Dispos. 42:1867-1872 (2014)).

“Human effector cells” are leukocytes that express one or more FcRs and perform effector functions. Preferably, the cells express at least FcγRIII and perform ADCC effector function. Examples of human leukocytes that mediate ADCC include PBMC, NK cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) that are bound to their cognate antigen. To assess complement activation, a CDC assay, e.g., as described in Gazzano-Santoro et al, J. Immunol. Methods 202:163 (1996), may be performed.

A “neutralizing antibody” is one that can neutralize the ability of that pathogen to initiate and/or perpetuate an infection in a host and/or in target cells in vitro. Described herein are neutralizing monoclonal human antibodies and antigen-binding fragments thereof, wherein the antibody recognizes an antigen from HIV, e.g., a gp120 polypeptide. In certain embodiments, a “neutralizing antibody” may inhibit the entry of HIV-1 virus, e.g., SF162 and/or JR-CSF, with a neutralization index>1.5 or >2.0 (Kostrikis L G et al./Virol. 1996; 70 (1): 445-458). By “broadly neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. In particular embodiments, a broad neutralizing antibody may neutralize multiple HIV-1 species belonging to at least 2, 3, 4, 5, or 6 different clades. In certain embodiments, the inhibitory concentration of the monoclonal antibody may be less than about 0.0001 μg/ml, less than about 0.001 μg/ml, less than about 0.01 μg/ml, less than about 0.1 μg/ml, less than about 0.5 μg/ml, less than about 1.0 μg/ml, less than about 5 μg/ml, less than about 10 μg/ml, less than about 25 μg/ml, less than about 50 μg/ml, or less than about 100 μg/ml to neutralize about 50% of the input virus in the neutralization assay.

HIV viruses are divided into specific groups, M, N, O and P, of which M is the “major” group and responsible for majority of HIV/AIDS globally. Based on their genetic sequence, Group M is further subdivided into subtypes (also called clades) with prevalence in distinct geographical locations.

A Group M “subtype” or “clade” is a subtype of HIV-1 group M defined by genetic sequence data. Examples of Group M subtypes include Subtypes A-K. Some of the subtypes are known to be more virulent or are resistant to different medications. There are also “circulating recombinant forms” or CRFs derived from recombination between viruses of different subtypes, which are each given a number. CRF12_BF, for example, is a recombination between subtypes B and F. Subtype A is common in West Africa. Subtype B is the dominant form in Europe, the Americas, Japan, Thailand, and Australia. Subtype C is the dominant form in Southern Africa, Eastern Africa, India, Nepal, and parts of China. Subtype D is generally only seen in Eastern and central Africa. Subtype E has never been identified as a nonrecombinant, only recombined with subtype A as CRF01_AE. Subtype F has been found in central Africa, South America and Eastern Europe. Subtype G (and the CRF02_AG) have been found in Africa and central Europe. Subtype H is limited to central Africa. Subtype I was originally used to describe a strain that is now accounted for as CRF04_cpx, with the cpx for a “complex” recombination of several subtypes. Subtype J is primarily found in North, Central and West Africa, and the Caribbean Subtype K is limited to the Democratic Republic of Congo and Cameroon. These subtypes are sometimes further split into sub-subtypes such as A1 and A2 or F1 and F2. In 2015, the strain CRF19, a recombinant of subtype A, subtype D and subtype G, with a subtype D protease was found to be strongly associated with rapid progression to AIDS in Cuba.

“HIV tropism” refers to the specificity of an HIV virus for a particular host cell, determined in part by the interaction of viral surface structures with receptors present on the surface of the host cell. HIV tropism of a patient's virus may be measured, e.g. by sequencing analysis or by the TROFILE® assay (monogrambio.com) (see, e.g., Lee, et al, AIDS Res Hum Retroviruses. (2013) 29 (6): 979-84).

HIV can infect a variety of cells such as CD4+ helper T cells and macrophages that express the CD4 molecule on their surface. HIV-1 entry to macrophages and T helper cells is mediated not only through interaction of the virion envelope glycoprotein, (e.g., gp120) with the CD4 molecule on the target cells but also with its chemokine coreceptors. Macrophage (M-tropic) strains of HIV-1, or non-syncitia-inducing strains (NSI) use the beta-chemokine receptor CCR5 for entry and are thus able to replicate in macrophages and CD4+ T-cells. These strains are called R5 viruses. This CCR5 coreceptor is used by almost all primary HIV-1 isolates regardless of viral genetic subtype. T-tropic isolates, or syncitia-inducing (SI) strains replicate in primary CD4+ T-cells as well as in macrophages and use the alpha-chemokine receptor, CSCR4, for entry. These strains are called X4 viruses. Viruses that use only the CCR5 receptor are termed R5, those that only use CXCR4 are termed X4, and those that use both, X4R5 or dual/mixed-tropism. However, the use of a coreceptor alone does not explain viral tropism, as not all R5 viruses are able to use CCR5 on macrophages for a productive infection.

Also described herein are “non-neutralizing antibodies,” which in certain embodiments are antibodies that bind to one or more strains of virus but do not neutralize the virus. However, in terms of Fc-mediated killing, the non-neutralizing antibody could still eliminate cells expressing viral antigens that are bound but not neutralized by the antibody. Thus, in certain embodiments, an antibody can bind a viral antigen and eliminate virally infected cells without neutralizing the virus.

The term “nucleic acid molecule” refers to a polymeric form of nucleotides and includes both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to

A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences described herein and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art.

The term “variant” may also refer to any naturally occurring or engineered molecule comprising one or more nucleotide or amino acid mutations. In one embodiment, the molecule is an antibody. For example, somatic variants may encompass all related naturally occurring antibodies that are part of or derived from the same B-cell lineage. Engineered variants may encompass all single mutations or combinatorial mutations made to an antibody.

Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed antibodies and antigen-binding fragments thereof, or corresponding DNA sequences that encode said polypeptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E. W. and Muller W. (1988) CABIOS 4:11-17; Robinson, E. D. (1971) Comb. Theor 77:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

“Homology” refers to the percentage of residues in the polynucleotide or polypeptide sequence variant that are identical to the non-variant sequence after aligning the sequences and introducing gaps, if necessary, to achieve the maximum percent homology.

“Binding affinity” may refer to a binding dissociate constant (Kd) or an apparent affinity (e.g., EC50) value.

### BRIEF DESCRIPTION OF THE DRAWINGS

FIG. 1 illustrates the number of screened subjects from the Zurich Primary HIV Infection Cohort Study with a genotype predicting sensitivity to GS-9722 (elipovimab). Pre-ART plasma samples from 92 individuals were analyzed in the GenoSure HIV Envelope RNA Assay. “None,” indicates all screened individuals without selection for specific amino acids in the HIV envelope gene. Amino acid positions indicated for each category.

FIG. 2 illustrates the number of screened clade B subjects from the Zurich Primary HIV Infection Cohort Study with a genotype predicting sensitivity to GS-9722. Pre-ART plasma samples from 59 clade B infected individuals were analyzed in the GenoSure HIV Envelope RNA Assay. “None,” indicates all screened individuals without selection for specific amino acids in the HIV envelope gene. Amino acid positions indicated for each category.

FIG. 3 illustrates the sensitivity to GS-9722 for swarm viruses derived from pre-ART plasma samples from the Zurich Primary HIV Infection Cohort Study. Virus from 29 samples with positive predictive values of 80.7% or higher were analyzed in the PHENOSENSE® HIV Entry Assay (Monogram Biosciences). Amino acid positions indicated for each category.

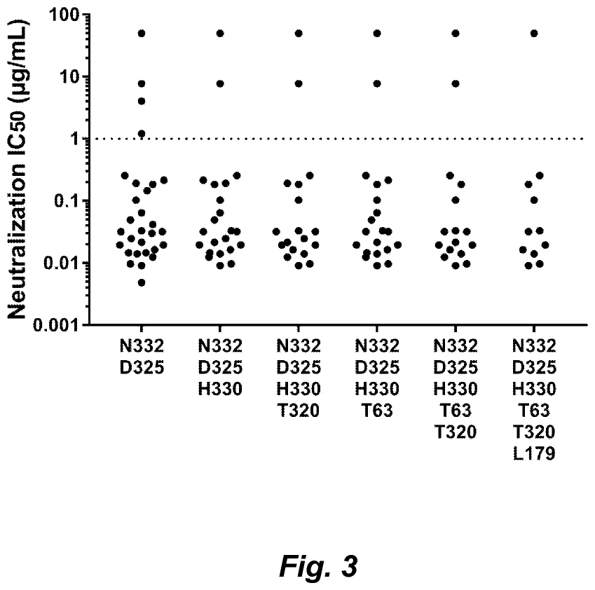

FIG. 4 illustrates the sensitivity to GS-9722 for viruses subcloned from swarm viruses derived from pre-ART plasma samples from the Zurich Primary HIV Infection Cohort Study. Twenty individual viruses from four pre-ART plasma samples, where swarm viruses were predicted sensitive by genotyping and tested sensitive by phenotyping, were analyzed in the PHENOSENSE® HIV Entry Assay (Monogram Biosciences). Solid line indicates IC50 for swarm virus.

### DETAILED DESCRIPTION

### 1. Introduction

The present methods are based, in part, on the unexpected discovery of HIV-infected patient populations who are responsive to the administration of an anti-HIV gp120 V3-glycan directed antibody or antigen-binding fragment thereof, in the absence of co-administration of additional anti-HIV antibodies directed against other HIV antigens (e.g., gp41) or non-overlapping epitopes of the same HIV antigen (e.g., directed against gp 120 in the region of the CD4 binding site or V2 apex region). Such patients are infected with a species of HIV having a gp 120 protein that is bound by a V3-glycan directed antibody or antigen-binding fragment thereof.

Generally, the methods entail identifying a human subject who is infected with an HIV or a population of HIV expressing a gp 120 comprising: a glycosylated asparagine at the position corresponding to amino acid residue position 332 (N332glycan), an aspartate at the position corresponding to amino acid residue position 325 (D325), and one or more amino acid of: a threonine at the position corresponding to amino acid residue position 63 (T63), a leucine at the position corresponding to amino acid residue position 179 (L179), a threonine at the position corresponding to amino acid residue position 320 (T320), and a histidine at the position corresponding to amino acid residue position 330 (H330), wherein the amino acid positions are with reference to SEQ ID NO: 4 (i.e., residues 1-511 of NCBI Ref Seq No. NP_057856.1). In various embodiments, the glycan is an oligomannose.

2. Identification of Subjects Responsive to Treatment with an Anti-HIV Gp120 V3-Glycan Directed Antibody or Antigen-Binding Fragment Thereof.

In some embodiments, the patient is identified by receiving a report of the HIV species infecting the patient that identifies the HIV gp120 amino acids residues present at the designated amino acid positions of interest, e.g., at positions 332 and 325, and one or more amino acid positions from the group consisting of: 63, 179, 320 and 330, wherein the amino acid positions are with reference to SEQ ID NO: 4. In some embodiments, the patient is identified by conducting one or more assays (e.g., polynucleotide or polypeptide sequencing) to determine the amino acid sequence(s) of the gp 120 or the amino acid residues present at the designated amino acid positions of interest of the gp 120 protein(s) of the HIV species infecting the patient. Identification of the full length or partial sequences of the gp 120 proteins obtained from the subject can be determined at the polynucleotide or polypeptide level. In some embodiments, the amino acids present at the gp 120 residue positions of interest are determined at the polypeptide level.

In various embodiments, the methods entail identifying a subject infected with an HIV or a population of HIV expressing a gp 120 comprising N332glycan, D325 and T63, wherein the amino acid positions are with reference to SEQ ID NO: 4.

In various embodiments, the methods entail identifying a subject infected with an HIV or a population of HIV expressing a gp 120 comprising N332glycan, D325 and L179, wherein the amino acid positions are with reference to SEQ ID NO: 4.

In various embodiments, the methods entail identifying a subject infected with an HIV or a population of HIV expressing a gp 120 comprising N332glycan, D325 and T320, wherein the amino acid positions are with reference to SEQ ID NO: 4.

In various embodiments, the methods entail identifying a subject infected with an HIV or a population of HIV expressing a gp 120 comprising N332glycan, D325 and H330, wherein the amino acid positions are with reference to SEQ ID NO: 4.

In various embodiments, the methods entail identifying a subject infected with an HIV or a population of HIV expressing a gp 120 comprising N332glycan, D325, T63 and L179, wherein the amino acid positions are with reference to SEQ ID NO: 4.

In various embodiments, the methods entail identifying a subject infected with an HIV or a population of HIV expressing a gp 120 comprising N332glycan, D325, T63 and T320, wherein the amino acid positions are with reference to SEQ ID NO: 4.

In some embodiments, the subject is infected with HIV clade B viruses. In various embodiments, the methods entail identifying a subject infected with an HIV or a population of HIV expressing a gp 120 comprising N332glycan, D325, T63 and H330, wherein the amino acid positions are with reference to SEQ ID NO: 4. In various embodiments, the methods entail identifying a subject infected with an HIV or a population of HIV expressing a gp 120 comprising N332glycan, D325, T63, L179, T320 and H330, wherein the amino acid positions are with reference to SEQ ID NO: 4.

In various embodiments, the methods entail identifying a subject infected with an HIV or a population of HIV expressing a gp120 comprising N332glycan, D325, T320 and H330, wherein the amino acid positions are with reference to SEQ ID NO: 4.

In various embodiments, the methods entail identifying a subject infected with an HIV or a population of HIV expressing a gp120 comprising N332glycan, D325, L179, T320 and H330, wherein the amino acid positions are with reference to SEQ ID NO: 4. In some embodiments, the subject is infected with HIV clade A and/or HIV clade C viruses. In some embodiments, the subject is infected with HIV clade A, clade B and/or HIV clade C viruses.

In various embodiments, the methods entail identifying a subject infected with an HIV or a population of HIV expressing a gp 120 comprising N332glycan, D325, T63, L179 and T320, wherein the amino acid positions are with reference to SEQ ID NO: 4.

In various embodiments, the methods entail identifying a subject infected with an HIV or a population of HIV expressing a gp 120 comprising N332glycan, D325, T63, L179 and H330, wherein the amino acid positions are with reference to SEQ ID NO: 4.

In some embodiments, the subject is infected with an HIV or a population of HIV expressing a gp 120 further comprising one or more of the following amino acid residues: a glycan at amino acid residue 301 (glycan301); a lysine at amino acid residue 677 (K677); an amino acid residue other than tryptophan (Trp, W) (e.g., alanine (Ala, A); cysteine (Cys, C); aspartate or aspartic acid (Asp, D); glutamate or glutamic acid (Glu, E); phenylalanine (Phe, F); glycine (Gly, G); histidine (His, H); isoleucine (Ile, I); lysine (Lys, K); leucine (Leu, L); methionine (Met, M); asparagine (Asn, N); proline (Pro, P); glutamine (Gln, Q); arginine (Arg, R); serine (Ser, S); Threonine (Thr, T); valine (Val, V) or tyrosine (Tyr, Y)) at position 17 (not_W17); an amino acid residue other than arginine (e.g., A, C, D, E, F, G, H, I, K, L, M, N, P, Q, S, T, V, W or Y) at position 747 (not_R747); an insertion_321.01 (e.g., an insertion of any amino acid (e.g., A, C, D, E, F, G, H, I, K, L, M, N, P, Q, R, S, T, V, W or Y) between position G321 and K322); a glutamic acid at position 429 (E429); a glutamine at position 442 (Q442); an arginine at position 335 (R335); an isoleucine at position 165 (1165); a serine at position 393 (S393); an isoleucine at position 307 (1307); a glycan at position 295 (295 glycan); and/or an asparagine at position 300 (N300), wherein the amino acid positions are with reference to SEQ ID NO: 4.

gp120

Envelope glycoprotein gp120 (or gp120) is a 120 kDa glycoprotein that is part of the outer layer of HIV. It presents itself as viral membrane spikes consisting of three molecules of gp120 linked together and anchored to the membrane by gp41 protein. Gp120 is essential for viral infection as it facilitates HIV entry into the host cell through its interaction with cell surface receptors. These receptors include DC-SIGN, Heparan Sulfate Proteoglycan, and the CD4 receptor. Binding to CD4 on helper T-cells induces the start of a cascade of conformational changes in gp 120 and gp41 that lead to the fusion of the virus with the host cell membrane.

Gp120 is encoded by the HIV env gene. The env gene encodes a gene product of around 850 amino acids. The primary env product is the protein gp160, which gets cleaved to gp120 (about 480 amino acids) and gp41 (about 345 amino acids) in the endoplasmic reticulum by the cellular protease furin.

The V3 glycan site on gp 120 is formed partly by a section of the CCR5 co-receptor site and partly by the surrounding camouflaging glycans (so-called “high mannose patch”) (Sok, et al., Immunity (2016) 45, 31-45). Broadly neutralizing antibodies (bnAbs) to the V3 glycan site are the most common of all Abs found in HIV infection (Walker, et al., PLOS Pathog. (2010) 6: e1001028 (2010); Landais, et al., PLOS Pathog. (2016) 12: e1005369; Georgiev, et al. Science (2013) 340:751-756). A consensus sequence of the V3 region of gp120 (Milich et al., J Virol., 67 (9): 5623-5634 (1993) is provided below:

(SEQ ID NO: 1)
CTRPNNNTRKSIHIGPGRAFYTTGEIIGDIRQAHC.

The amino acid sequence of an exemplary gp160 polypeptide of HIV clone WITO is provided below (the V3 hypervariable loop is boldened and the N332 potential N-linked glycosylation site is boldened and underlined):

(SEQ ID NO: 2)
MKVMGTKKNYQHLWRWGIMLLGMLMMSSAAEQLWVTVYYGVPVWREANTT
LFCASDAKAYDTEVHNVWATHACVPTDPNPQEVVMGNVTEDFNMWKNNMV
EQMHEDIISLWDQSLKPCVKLTPLCVTLHCTNVTISSTNGSTANVIMREE
MKNCSFNTTTVIRDKIQKEYALFYKLDIVPIEGKNTNTSYRLINCNTSVI
TQACPKVSFEPIPIHYCAPAGFAILKCNNKTFNGKGPCRNVSTVQCTHGI
KPVVSTQLLLNGSLAEEDIIIRSENFTNNGKNIIVQLKEPVKINCTRPGN
NTRRSINIGPGRAFYATGAIIGDIRKAHCNISTEQWNNTLTQIVDKLREQ
FGNKTIIFNQSSGGDPEVVMHTFNCGGEFFYCNSTQLFNSTWFNNGTSTW
NSTADNITLPCRIKQVINMWQEVGKAMYAPPIRGQIDCSSNITGLILTRD
GGSNSSQNETFRPGGGNMKDNWRSELYKYKVVKIEPLGIAPTRAKRRVVQ
REKRAVTLGAVFLGFLGAAGSTMGAASLTLIVQARLLLSGIVQQQSNLLR
AIEAQQHMLQLTVWGIKQLQARVLAIERYLKDQQLLGIWGCSGKLICTTT
VPWNTSWSNKSYDYIWNNMTWMQWEREIDNYTGFIYTLIEESQNQQEKNE
LELLELDKWASLWNWFNITNWLWYIKLFIMIIGGLVGLRIVCAVLSIVNR
VRQGYSPLSFQTRLPNPRGPDRPEETEGEGGERDRDRSARLVNGFLAIIW
DDLRSLCLFSYHRLRDLLLIVARVVEILGRRGWEILKYWWNLLKYWSQEL
KNSAVSLLNVTAIAVAEGTDRVIEIVQRAVRAILHIPTRIRQGFERALL

The amino acid sequence of an exemplary gp160 polypeptide of HIV clone identified in NCBI Ref Seq No. NP_057856.1 is provided below (the V3 hypervariable loop is boldened and the N332 potential N-linked glycosylation site is boldened and underlined):

(SEQ ID NO: 3)
MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLTSCNTSV
ITQACPKVSFEPIPIHYCAPAGFAILKCNNKTFNGTGPCTNVSTVQCTHG
IRPVVSTQLLLNGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINMWQKVGKAMYAPPISGQIRCSSNIT
GLLLTRDGGNSNNESEIFRPGGGDMRDNWRSELYKYKVVKIEPLGVAPTK
AKRRVVQREKRAVGIGALFLGFLGAAGSTMGAASMTLTVQARQLLSGIVQ
QQNNLLRAIEAQQHLLQLTVWGIKQLQARILAVERYLKDQQLLGIWGCSG
KLICTTAVPWNASWSNKSLEQIWNHTTWMEWDREINNYTSLIHSLIEESQ
NQQEKNEQELLELDKWASLWNWFNITNWLWYIKLFIMIVGGLVGLRIVFA
VLSIVNRVRQGYSPLSFQTHLPTPRGPDRPEGIEEEGGERDRDRSIRLVN
GSLALIWDDLRSLCLFSYHRLRDLLLIVTRIVELLGRRGWEALKYWWNLL
QYWSQELKNSAVSLLNATAIAVAEGTDRVIEVVQGACRAIRHIPRRIRQG
LERILL

The amino acid sequence of an exemplary gp120 polypeptide of HXB2 subtype B HIV-1 isolate (GenBank Accession No. K0345; corresponding to residues 1-511 of NCBI Ref Seq No. NP_057856.1) is provided below (the V3 hypervariable loop is boldened and the N332 potential N-linked glycosylation site is boldened and underlined; signal peptide is underlined):

(SEQ ID NO: 4)
MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLTSCNTSV
ITQACPKVSFEPIPIHYCAPAGFAILKCNNKTFNGTGPCTNVSTVQCTHG
IRPVVSTQLLLNGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINMWQKVGKAMYAPPISGQIRCSSNIT
GLLLTRDGGNSNNESEIFRPGGGDMRDNWRSELYKYKVVKIEPLGVAPTK
AKRRVVQREKR

The amino acid sequence of an exemplary gp120 polypeptide is provided below:

(SEQ ID NO: 5)
AEQLWVTVYYGVPVWREANTTLFCASDAKAYDTEVHNVWATHACVPTDPN
PQEVVMGNVTEDFNMWKNNMVEQMHEDIISLWDQSLKPCVKLTPLCVTLH
CTNVTISSTNGSTANVTMREEMKNCSFNTTTVIRDKIQKEYALFYKLDIV
PIEGKNTNTSYRLINCNTSVITQACPKVSFEPIPIHYCAPAGFAILKCNN
KTFNGKGPCRNVSTVQCTHGIKPVVSTQLLLNGSLAEEDIIIRSENFTNN
GKNIIVQLKEPVKINCTRPGNNTRRSINIGPGRAFYATGAIIGDIRKAHC
NISTEQWNNTLTQIVDKLREQFGNKTIIFNQSSGGDPEVVMHTFNCGGEF
FYCNSTQLFNSTWFNNGTSTWNSTADNITLPCRIKQVINMWQEVGKAMYA
PPIRGQIDCSSNITGLILTRDGGSNSSQNETFRPGGGNMKDNWRSELYKY
KVVKIEPLGIAPTRAKRRVVQREKR.

The amino acid sequence of another exemplary gp120 polypeptide (see, bioafrica.net/proteomics/ENV-GP120prot.html) is provided below:

(SEQ ID NO: 6)
TEKLWVTVYY GVPVWKEATT TLFCASDAKA YDTEVHNVWA
THACVPTDPN PQEVVLVNVT ENFNMWKNDM VEQMHEDIIS
LWDQSLKPCV KLTPLCVSLK CTDLKNDTNT NSSSGRMIME
KGEIKNCSFN ISTSIRGKVQ KEYAFFYKLD IIPIDNDTTS
YKLTSCNTSV ITQACPKVSF EPIPIHYCAP AGFAILKCNN
KTFNGTGPCT NVSTVQCTHG IRPVVSTQLL LNGSLAEEEV
VIRSVNFTDN AKTIIVQLNT SVEINCTRPN NNTRKRIRIQ
RGPGRAPVTI GKIGNMRQAH CNISRAKWNN TLKQIASKLR
EQFGNNKTII FKQSSGGDPE IVTHSFNCGG EFFYCNSTQL
FNSTWFNSTW STEGSNNTEG SDTITLPCRI KQIINMWQKV
GKAMYAPPIS GQIRCSSNIT GLLLTRDGGN SNNESEIFRP
GGGDMRDNWR SELYKYKVVK IEPLGVAPTK AKRRVVQREK R

Genomic diversity among independent human immunodeficiency virus type 1 (HIV-1) isolates, to a lesser degree among sequential isolates from the same patients, and even within a single patient isolate is a well-known feature of HIV-1. Although this sequence heterogeneity is distributed throughout the genome, most of the heterogeneity is located in the env gene. Comparison of predicted amino acid sequences from several different isolates has shown that sequence heterogeneity is clustered in five variable regions (designated V1 through V5) of the surface glycoprotein, gp120. The V3 region, although only 35 amino acids long, exhibits considerable sequence variability. Interestingly, despite this variability, the V3 region includes determinants that mediate interactions with CD4⁺ cells. The increase in gp120 variability results in higher levels of viral replication, suggesting an increase in viral fitness in individuals infected by diverse HIV-1 variants. Variability in potential N-linked glycosylation sites (PNGSs) also result in increased viral fitness. PNGSs allow for the binding of long-chain carbohydrates to the high variable regions of gp120. Thus, the number of PNGSs in env might affect the fitness of the virus by providing more or less sensitivity to neutralizing antibodies.

### Biological Sample

The HIV gp120 amino acid residues of interest are determined from HIV present or suspected to be present in a biological sample from the subject. The biological sample can be from a solid tissue or biological fluid of the subject known or suspected to contain HIV. In various embodiments, the biological sample comprises or is from blood, peripheral blood mononuclear cells (PBMCs), serum, plasma, semen or lymph nodes. In some embodiments, the biological sample comprises or is from bile, blood, blood plasma, serum, breast milk, feces, pus, saliva, sebum, semen, sweat, tears, urine, or vomit. In patients whose virus levels are suppressed, e.g., by antiretroviral (ART) therapy, the biological sample comprises solid tissue or biological fluid of the subject known or suspected to contain an HIV reservoir, e.g., solid tissues and/or biological fluids comprising latently HIV-infected CD4+ T cells (including memory and non-memory effector CD4+ T cells), hematopoietic progenitors of CD4+ T cells, γδT cells (including memory and non-memory effector γδT cells), natural killer (NK) cells, myeloid cells (including monocytes and macrophages), hematopoietic progenitors of myeloid cells and follicular dendritic cells. Anatomical reservoirs that may harbor latently HIV-infected cells include lymphoid tissues, the brain and the central nervous system, the gastrointestinal tract and the gut-associated lymphoid tissue (GALT), genital tract, lungs and skin. Tissues and cells found to harbor latently HIV infected cells and HIV reservoirs are described, e.g., in Kuo, et al., Curr Opin HIV AIDS. (2018) 13 (2): 137-142; Mzingwane, et al., Rev Med Virol. (2017) March; 27 (2), doi: 10.1002/rmv.1924 (PMID 28128885); Churchill, et al., Nat Rev Microbiol. (2016) 14 (1): 55-60; Barton, et al., Trends Microbiol. (2016) 24 (5): 345-355, which are hereby incorporated herein by reference in their entireties for all purposes.

In some embodiments, multiple biological samples are evaluated from a single patient. For example, in some embodiments two or more biological samples from two or more different tissues or two or more different anatomical reservoirs are evaluated from a single patient.

### Stage of Infection

In various embodiments, the human subject is an adult, a juvenile or an infant. The subject may be symptomatic (e.g., viremic) or asymptomatic (e.g., acutely infected or ART suppressed). In some embodiments, the human subject is acutely infected or recently infected with HIV. In certain embodiments, the subject has not seroconverted. In some embodiments, the human subject is chronically infected with HIV. The subject many or may not be receiving a regimen of antiretroviral therapy (ART).

Patients can be categorized into Fiebig stages I-VI, which are based on a sequential gain in positive HIV-1 clinical diagnostic assays (viral RNA measured by PCR, p24 and p31 viral antigens measured by enzyme-linked immunosorbent assay (ELISA). p24 antigen is a viral core protein that transiently appears in the blood during the ramp-up phase once HIV-1 RNA levels rise above 10,000 copies/mL and before the development of detectable HIV antibodies. In Fiebig stage I, during ramp-up viremia, only HIV-1 RNA in the blood can be detected. Fiebig stage II commences about 7 days later, when results of tests to detect p24 antigen become positive. In Fiebig stage III, within about 5 days after p24 antigen test results become positive, IgM anti-HIV-1 antibodies can be detected with sufficiently sensitive enzyme immunoassays (EIAs) (e.g., third-generation EIAs). Stage III typically occurs 1-2 weeks after the onset of acute retroviral symptoms. Fiebig stage IV represents the development of an indeterminate Western blot test and occurs about 3 days after EIA tests show positive results. Conversion to a clearly positive Western blot test, Fiebig stage V, generally occurs after another 7 days, or about 1 month after initial infection. Fiebig stages of HIV infection are described, e.g., in Fiebig, et al., AIDS. (2003) 17 (13): 1871-9; Cohen, et al., J Infect Dis. (2010) 202 Suppl 2: S270-7; and McMichacl, et al., Nature Reviews Immunology (2010) 10:11-23, which are hereby incorporated herein by reference in their entireties for all purposes. In some embodiments, the biological sample evaluated is from a human subject having an HIV infection of Fiebig stage IV or earlier, e.g., Fiebig stage I, Fiebig stage II, Fiebig stage III or Fiebig stage IV. In some embodiments, the biological sample evaluated is from a human subject having an HIV infection of an HIV infection of Fiebig stage V or Fiebig stage VI.

In some embodiments, the methods further comprise the step of obtaining the biological sample from the subject. In some embodiments, the methods entail receiving a report of the HIV gp120 amino acids residues present at the designated positions of interest, e.g., at 332 and 325, and one or more amino acid positions from the group consisting of: 63, 179, 320 and 330, wherein the amino acid positions are with reference to SEQ ID NO: 4.

### Determining Gp120 Amino Acids of Interest

Determination of the amino acid residues at HIV gp120 sequences of a subject at the designated positions of interest, e.g., at 332 and 325, and one or more amino acid positions from the group consisting of: 63, 179, 320 and 330, wherein the amino acid positions are with reference to SEQ ID NO: 4, can be done at the polynucleotide or polypeptide level. At the level of the polynucleotide, HIV RNA or proviral DNA isolated from one or more biological samples can be sequenced using methods known in the art. In some embodiments, HIV RNA or proviral DNA isolated from two or more biological samples of a subject are sequenced. In some embodiments, the two or more biological samples are obtained from different tissue sources (e.g., blood, peripheral blood mononuclear cells, lymph nodes and/or semen). In some embodiments, the two or more biological samples are obtained at different time points, e.g., 1, 2, 3, 4, 5, 6, 7 or 8 weeks apart, 3, 4, 5, 6, 7, 8, 9, 10, 11 or 12 months apart.

As appropriate, primers that anneal to and amplify the HIV env coding sequence, and particularly the V3 variable region of gp 120, can be used. In some embodiments, nested sets of primers can be used. In various embodiments, the RNA is sequenced directly or reverse-transcriptase polymerase chain reaction (RT-PCR) can be performed. In some embodiments, Sanger sequencing can be performed, e.g., when sequencing to determine amino acid residues in the V3 region, or when sequencing a sample from a patient in an early Fiebig stage of disease, e.g., prior to Fiebig stage III, e.g., Fiebig stages I or II. In various embodiments, single genome amplification (SGA) and sequencing is performed. Methods for single genome amplification (SGA) and sequencing of plasma HIV virion RNA, are described, e.g., in Salazar-Gonzalez, et al. (2008) J Virol 82:3952-3970; and Keele, et al., Proc Natl Acad Sci USA. (2008) 105 (21): 7552-7. Application of SGA to determining amino acid sequence variance in HIV gp120 sequences, and which can be employed in the herein described methods, is described, e.g., in Bar, et al., N Engl J Med. (2016) 375 (21): 2037-2050; and Mendoza, et al., Nature. (2018) 561 (7724): 479-484. In various embodiments, high throughput, Next Generation Sequencing (NGS), massively parallel or deep sequencing techniques are employed to sequence gp120, including at least the V3 variable region, from a population of HIV species in one or more biological samples from a single patient or subject. In such cases, multiple nucleic acid sequences encoding at least the V3 variable region of gp 120 are sequenced and aligned. In some embodiments, the full-length of gp120 is sequenced. Illustrative platforms for performing NGS sequencing that can be used for determining the gp 120 sequences of HIV species in one or more biological samples from a patient include Illumina (Solexa) (illumina.com), Ion torrent: Proton/PGM sequencing (thermofisher.com), SOLID (thermofisher.com), and Single Molecule, Real-Time (SMRT) Sequencing (Pacific Biosciences, pacb.com). Methods for isolating and sequencing HIV gp120, including at least the V3 glycan region, from patients, and which can be applied in the present methods, are described in, e.g., Shioda, et al., J Virol. (1997) 71 (7): 4871-81; Colón, et al., J Virol Antivir Res. (2015) 4 (3). pii: 143 (PMID: 27358904); Kafando, et al., PLOS One. (2017) 12 (12): c0189999; Hebberecht, et al., PLOS One. (2018) 13 (4): c0195679, Andrews, et al., Sci Rep. (2018) 8 (1): 5743 and Landais, et al. Immunity. (2017) 47 (5): 990-1003. As appropriate, shorter sequence reads of the nucleic acid sequences (“contigs”) can be assembled into longer sequences, including at least the V3 variable region of gp120. Methods of contig assembly of HIV genomic sequences that can be applied in the present methods are described, e.g., in Huang, et al., Bioinformation. (2018) 14 (8): 449-454; Hiener, et al., J Vis Exp. (2018) October 16; (140). doi: 10.3791/58016; and Wymant, et al., Virus Evol. (2018) May 18; 4 (1): vey007. doi: 10.1093/ve/vey007.

In some embodiments, at least 90%, at least 91%, at least 92%, at least 93%, at least 94%, at least 95%, at least 96%, at least 97%, at least 98%, at least 99%, or 100%, of the sequenced V3 variable region of gp 120 in a population of HIV obtained from one or more biological samples in a single patient comprise an amino acid sequence comprising a glycosylated asparagine at the position corresponding to amino acid residue position 332 (N332glycan), an aspartate at the position corresponding to amino acid residue position 325 (D325), and one or more of a threonine at the position corresponding to amino acid residue position 63 (T63), a leucine at the position corresponding to amino acid residue position 179 (L179), a threonine at the position corresponding to amino acid residue position 320 (T320), and a histidine at the position corresponding to amino acid residue position 330 (H330), wherein the amino acid positions are with reference to SEQ ID NO: 4. As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer. In some embodiments, HIV gp 120 variants are detected to a frequency level about 1% (e.g., 1% mutant or variant frequency) of the virus population. In some embodiments, HIV gp120 variants are detected to a frequency level of about 0.5% of the virus population. As a rule of thumb, reliable detection of variants at 1% frequency will require HIV RNA levels of at least 1000 copies/mL. See, e.g., Casadellà, et al., Virus Research 239 (2017) 69-81; Noguera-Julian, et al., J Infect Dis. (2017) 216 (suppl_9): S829-S833 and Lee, et al., Sci Rep. (2020) 10 (1): 1634.

### 3. Administration of an Anti-HIV Gp120 V3-Glycan Directed Antibody or Antigen-Binding Fragment Thereof

In certain embodiments, the methods entail administration of an anti-HIV antibody or antigen-binding fragment thereof, or antigen binding molecule, that targets the V3 glycan binding region of gp120.

HIV-1 is the main family of HIV and accounts for 95% of all infections worldwide. HIV-2 is mainly seen in a few West African countries.

HIV viruses are divided into specific groups, M, N, O and P, of which M is the “major” group and responsible for majority of HIV/AIDS globally. Based on their genetic sequence, Group M is further subdivided into subtypes (also called clades) with prevalence in distinct geographical locations.

A Group M “subtype” or “clade” is a subtype of HIV-1 group M defined by genetic sequence data. Examples of Group M subtypes include Subtypes A-K. Some of the subtypes are known to be more virulent or are resistant to different medications. There are also “circulating recombinant forms” or CRFs derived from recombination between viruses of different subtypes, which are each given a number. CRF12_BF, for example, is a recombination between subtypes B and F. Subtype A is common in West Africa. Subtype B is the dominant form in Europe, the Americas, Japan, Thailand, and Australia. Subtype C is the dominant form in Southern Africa, Eastern Africa, India, Nepal, and parts of China. Subtype D is generally only seen in Eastern and central Africa. Subtype E has never been identified as a nonrecombinant, only recombined with subtype A as CRF01_AE. Subtype F has been found in central Africa, South America and Eastern Europe. Subtype G (and the CRF02_AG) have been found in Africa and central Europe. Subtype H is limited to central Africa. Subtype I was originally used to describe a strain that is now accounted for as CRF04_cpx, with the cpx for a “complex” recombination of several subtypes. Subtype J is primarily found in North, Central and West Africa, and the Caribbean Subtype K is limited to the Democratic Republic of Congo and Cameroon. These subtypes are sometimes further split into sub-subtypes such as A1 and A2 or F1 and F2. In 2015, the strain CRF19, a recombinant of subtype A, subtype D, and subtype G, with a subtype D protease was found to be strongly associated with rapid progression to AIDS in Cuba.

This disclosure provides, inter alia, methods entailing administration of human anti-HIV neutralizing antibodies (e.g., broadly neutralizing Abs) that target the V3-Glycan region of the gp120 polypeptide on the surface of HIV-infected cells. Neutralizing antibodies against viral envelope proteins provide adaptive immune defense against HIV-1 exposure by blocking the infection of susceptible cells. Broad neutralization indicates that the antibodies can neutralize HIV-1 isolates from different clades. Thus, the anti-HIV gp120 V3 glycan directed antibodies or antigen-binding fragments described herein have cross-clade binding activity.

### Antibodies and Antigen-Binding Fragments Thereof Directed to the V3 Glycan Region of HIV Gp120

In certain embodiments of the methods described herein, the subject is administered an antibody or antigen-binding fragment thereof, or an antigen-binding molecule that binds to HIV gp120 protein within the V3 Glycan region, e.g., an epitope or region of gp120 third variable loop (V3) and/or high mannose patch comprising a N332 oligomannose glycan. In certain embodiments, the administered antibody or antigen-binding fragment thereof, or an antigen-binding molecule binds to HIV-1 antigens expressed on a cell surface and eliminates or kills the infected cell.

In certain embodiments, the administered antibody or antigen-binding fragment thereof, or an antigen-binding molecule, is or is derived from human neutralizing antibodies (e.g., monoclonal) that target HIV-1. A “neutralizing antibody” is one that can neutralize the ability of HIV to initiate and/or perpetuate an infection in a host and/or in target cells in vitro. The disclosure provides neutralizing monoclonal human antibodies, wherein the antibody recognizes an antigen from HIV, e.g., a gp 120 polypeptide. In certain embodiments, a “neutralizing antibody” may inhibit the entry of HIV-1 virus, e.g., SF162 and/or JR-CSF, with a neutralization index>1.5 or >2.0 (Kostrikis L G et al., J. Virol., 70 (1): 445-458 (1996)).

In some embodiments, the administered antibody or antigen-binding fragment thereof, or an antigen-binding molecule, is or is derived from human broadly neutralizing antibodies (e.g., monoclonal) that target HIV-1. By “broadly neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. In particular embodiments, a broad neutralizing antibody may neutralize multiple HIV-1 species belonging to at least 2, 3, 4, 5, or 6 different clades. In certain embodiments, the inhibitory concentration of the anti-HIV gp120 V3 glycan directed antibody or antigen-binding fragment may be less than about 0.0001 μg/ml, less than about 0.001 μg/ml, less than about 0.01 μg/ml, less than about 0.1 μg/ml, less than about 0.5 μg/ml, less than about 1.0 μg/ml, less than about 5 μg/ml, less than about 10 μg/ml, less than about 25 μg/ml, less than about 50 μg/ml, or less than about 100 μg/ml to neutralize about 50% of the input virus in the neutralization assay.

Illustrative broadly neutralizing antibodies that bind to gp 120 in the third variable loop (V3) and/or high mannose patch comprising a N332 oligomannose glycan and which can be used in the herein described methods include without limitation GS-9722 (clipovimab), GS-9721, PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp 120 in the third variable loop (V3) and/or high mannose patch comprising a N332 oligomannose glycan and which can be used in the herein described methods are described, e.g., in WO 2012/030904; WO 2014/063059; WO 2016/149698; WO 2017/106346; WO 2018/075564, WO 2018/125813; WO 2018/237148, WO 2019/226829, WO 2020/023827, WO2020/056145 and Kerwin, et al., J Pharm Sci. 2020 January; 109 (1): 233-246, which are hereby incorporated herein by reference in their entireties for all purposes.

Illustrative sequences of complementarity determining regions (CDRs) of the antibody or antigen-binding fragments, targeting HIV gp120 V3 glycan region, are provided in Tables A1-A4. Illustrative sequences of the VH and VL of the antibody or antigen-binding fragments, targeting HIV gp120 V3 glycan region, are provided in Table B.

TABLE A1
CDRs (Kabat) for Anti-HIV gp120 V3 Glycan-Directed Antibodies or Antigen-Binding Fragments
Thereof

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 1 | DSYWS | YVHKSGDTNYSPSLKS | TLHGRRIYGIVAFNEW | GEKSLGSRAVQ | NNQDRPS | HIWDSRVPTK |
| | SEQ ID NO: 7 | SEQ ID NO: 8 | FTYFYMDV | SEQ ID | SEQ ID | WV |
| | | | SEQ ID NO: 9 | NO: 10 | NO: 11 | SEQ ID |
| | | | | | | NO: 12 |
| 2 | DSYWS | YVHKSGDTNYNPSLKS | TLHGRRIYGIVAFNEW | GEKSLGSRAVQ | NNQDRPS | HIWDSRVPTK |
| | SEQ ID NO: 7 | SEQ ID NO: 13 | FTYFYMDV | SEQ ID | SEQ ID | WV |
| | | | SEQ ID NO: 9 | NO: 10 | NO: 11 | SEQ ID |
| | | | | | | NO: 12 |
| 3 | NYYWT | YISDRESATYNPSLNS | ARRGQRIYGVVSFGEF | GRQALGSRAVQ | NNQDRPS | HMWDSRSGFS |
| | SEQ ID | SEQ ID NO: 15 | FYYYSMDV | SEQ ID | SEQ ID | WS |
| | NO: 14 | | SEQ ID NO: 16 | NO: 17 | NO: 11 | SEQ ID |
| | | | | | | NO: 18 |
| 4 | NYYWT | YISDRETTTYNPSLNS | ARRGQRIYGVVSFGEF | GRQALGSRAVQ | NNQDRPS | HMWDSRSGFS |
| | SEQ ID | SEQ ID NO: 19 | FYYYYMDV | SEQ ID | SEQ ID | WS |
| | NO: 14 | | SEQ ID NO: 20 | NO: 17 | NO: 11 | SEQ ID |
| | | | | | | NO: 18 |
| 5 | GRFWS | YFSDTDRSEYNPSLRS | AQQGKRIYGIVSFGEF | GERSRGSRAVQ | NNQDRPA | HYWDSRSPIS |
| | SEQ ID | SEQ ID NO: 22 | FYYYYMDA | SEQ ID | SEQ ID | WI |
| | NO: 21 | | SEQ ID NO: 23 | NO: 24 | NO: 25 | SEQ ID |
| | | | | | | NO: 26 |
| 6 | GRFWS | YFSDTDRSEYNPSLRS | AQQGKRIYGIVSFGEL | GERSRGSRAVQ | NNQDRPA | HYWDSRSPIS |
| | SEQ ID | SEQ ID NO: 22 | FYYYYMDA | SEQ ID | SEQ ID | WI |
| | NO: 21 | | SEQ ID NO: 27 | NO: 24 | NO: 25 | SEQ ID |
| | | | | | | NO: 26 |
| 7 | DNYWS | YVHDSGDTNYNPSLKS | TKHGRRIYGVVAFKEW | GEESLGSRSVI | NNNDRPS | HIWDSRRPTN |
| | SEQ ID | SEQ ID NO: 29 | FTYFYMDV | SEQ ID | SEQ ID | WV |
| | NO: 28 | | SEQ ID NO: 30 | NO: 31 | NO: 32 | SEQ ID |
| | | | | | | NO: 33 |
| 8 | DAYWS | YVHHSGDTNYNPSLKR | ALHGKRIYGIVALGEL | GKESIGSRAVQ | NNQDRPA | HIYDARGGTN |
| | SEQ ID | SEQ ID NO: 35 | FTYFYMDV | SEQ ID | SEQ ID | WV |
| | NO: 34 | | SEQ ID NO: 36 | NO: 37 | NO: 25 | SEQ ID |
| | | | | | | NO: 38 |
| 9 | ACTYFWG | SLSHCQSFWGSGWTFH | FDGEVLVYNHWPKPAW | NGTATNFVS | GVDKRPP | GSLVGNWDVI |
| | SEQ ID | NPSLKS | VDL | SEQ ID | SEQ ID | SEQ ID |
| | NO: 39 | SEQ ID NO: 40 | SEQ ID NO: 41 | NO: 42 | NO: 43 | NO: 44 |
| 10 | ACDYFWG | GLSHCAGYYNTGWTYH | FDGEVLVYHDWPKPAW | TGTSNRFVS | GVNKRPS | SSLVGNWDVI |
| | SEQ ID | NPSLKS | VDL | SEQ ID | SEQ ID | SEQ ID |
| | NO: 45 | SEQ ID NO: 46 | SEQ ID NO: 47 | NO: 48 | NO: 49 | NO: 50 |
| 11 | ACDYFWG | SLSHCAGYYNSGWTYH | FGGDVLVYHDWPKPAW | TGNINNFVS | GVNKRPS | GSLAGNWDVV |
| | SEQ ID | NPSLKS | VDL | SEQ ID | SEQ ID | SEQ ID |
| | NO: 45 | SEQ ID NO: 51 | SEQ ID NO: 52 | NO: 53 | NO: 49 | NO: 54 |
| 12 | ACNSFWG | SLSHCASYWNRGWTYH | FGGEVLRYTDWPKPAW | TGTSNNFVS | DVNKRPS | GSLVGNWDVI |
| | SEQ ID | NPSLKS | VDL | SEQ ID | SEQ ID | SEQ ID |
| | NO: 55 | SEQ ID NO: 56 | SEQ ID NO: 57 | NO: 58 | NO: 59 | NO: 44 |
| 13 | GCDYFWG | GLSHCAGYYNTGWTYH | FDGEVLVYNDWPKPAW | TGTSNNFVS | GVNKRPS | GSLVGNWDVI |
| | SEQ ID | NPSLKS | VDL | SEQ ID | SEQ ID | SEQ ID |
| | NO: 61 | SEQ ID NO: 46 | SEQ ID NO: 63 | NO: 58 | NO: 49 | NO: 44 |
| 14 | TGHYYWG | HIHYTTAVLHNPSLKS | SGGDILYYYEWQKPHW | NGTSSDIGGWN | EVNKRPS | SSLFGRWDVV |
| | SEQ ID | SEQ ID NO: 65 | FSP | FVS | SEQ ID | SEQ ID |
| | NO: 64 | | SEQ ID NO: 66 | SEQ ID | NO: 68 | NO: 69 |
| | | | | NO: 67 | | |
| 15 | GTDWGENDFHY | SIHWRGRTTHYKTSFR | HKYHDIFRVVPVAGWF | RASQNVKNNLA | DASSRAG | QQYEEWPRT |
| | G | S | DP | SEQ ID | SEQ ID | SEQ ID |
| | SEQ ID | SEQ ID NO: 71 | SEQ ID NO: 72 | NO: 73 | NO: 74 | NO: 75 |
| | NO: 70 | | | | | |
| 16 | GGEWGDSDYHW | SIHWRGTTHYNAPFRG | HKYHDIVMVVPIAGWF | RASQSVKNNLA | DTSSRAS | QQYEEWPRT |
| | G | SEQ ID NO: 77 | DP | SEQ ID | SEQ ID | SEQ ID |
| | SEQ ID | | SEQ ID NO: 78 | NO: 79 | NO: 80 | NO: 75 |
| | NO: 76 | | | | | |
| 17 | GGEWGDKDYHW | SIHWRGTTHYKESLRR | HRHHDVFMLVPIAGWF | RASQNINKNLA | ETYSKIA | QQYEEWPRT |
| | G | SEQ ID NO: 82 | DV | SEQ ID | SEQ ID | SEQ ID |
| | SEQ ID | | SEQ ID NO: 83 | NO: 84 | NO: 62 | NO: 75 |
| | NO: 81 | | | | | |
| 18 | SDHSWT | DIHYNGATTYNPSLRS | NAIRIYGVVALGEWFH | SGAPLTSRFTY | RSSQRSS | QSSDTSDSYK |
| | SEQ ID | SEQ ID NO: 86 | YGMDV | SEQ ID | SEQ ID | M |
| | NO: 85 | | SEQ ID NO: 87 | NO: 88 | NO: 89 | SEQ ID |
| | | | | | | NO: 90 |
| 19 | SDHSWT | DVHYNGDNTYNPSLRG | NVIRVFGVISLGEWFH | SGPPLASRYTY | RDRQFPS | QSSDTSDSYK |
| | SEQ ID | SEQ ID NO: 91 | YGMDV | SEQ ID | SEQ ID | M |
| | NO: 85 | | SEQ ID NO: 92 | NO: 93 | NO: 94 | SEQ ID |
| | | | | | | NO: 90 |
| 20 | SDHSWT | DVHYNGDTTYNPSLRG | NVIRVFGVISLGEWFH | SGPPLASRYTY | RDRQFPS | QSSDTSDSYK |
| | SEQ ID | SEQ ID NO: 96 | YGMDV | SEQ ID NO: | SEQ ID | M |
| | NO: 85 | | SEQ ID NO: 92 | 93 | NO: 94 | SEQ ID |
| | | | | | | NO: 90 |
| 21 | SDHSWT | DIHYNGATTYNPSLRS | NAIRIYGVVALGEWFH | SGAALTSRFTY | RTSQRSS | QSSDTSDSYK |
| | SEQ ID | SEQ ID NO: 86 | YGMDV | SEQ ID | SEQ ID | M |
| | NO: 85 | | SEQ ID NO: 87 | NO: 97 | NO: 98 | SEQ ID |
| | | | | | | NO: 90 |
| 22 | SDHSWT | DIHYGGDITYNPSLRS | NVIRVFGVIALGEWFH | SGPPLASRYCY | RDRQFSS | QSSDINDSYK |
| | SEQ ID | SEQ ID NO: 99 | YGMDV | SEQ ID | SEQ ID | M |
| | NO: 85 | | SEQ ID NO: 100 | NO: 101 | NO: 102 | SEQ ID |
| | | | | | | NO: 95 |
| 23 | SDHSWT | DIHYGGDITYNPSLRS | NVIRVFGVIALGEWFH | SGPPLASRYCY | RDRQFSS | QSSDTSDSFK |
| | SEQ ID | SEQ ID NO: 99 | YGMDV | SEQ ID | SEQ ID | M |
| | NO: 85 | | SEQ ID NO: 100 | NO: 101 | NO: 102 | SEQ ID |
| | | | | | | NO: 103 |
| 24 | SDHSWT | DIHYGGDITYNPSLRS | NVIRVFGVIALGEWFH | SGPPLATRYCY | RDRQFSS | QSSDTSDSYK |
| | SEQ ID | SEQ ID NO: 99 | YGMDV | SEQ ID | SEQ ID | M |
| | NO: 85 | | SEQ ID NO: 100 | NO: 104 | NO: 102 | SEQ ID |
| | | | | | | NO: 90 |
| 25 | SDHSWT | DIHYNGDKTYNPSLRG | NVIRVFGVISLGEWFH | SGPPLASRYTY | RDRQFPS | QSSDTSDSYK |
| | SEQ ID | SEQ ID NO: 105 | YGMDV | SEQ ID | SEQ ID | M |
| | NO: 85 | | SEQ ID NO: 92 | NO: 93 | NO: 94 | SEQ ID |
| | | | | | | NO: 90 |
| 26 | SDHSWT | DIHYGGDITYNPSLRS | NVIRVFGVIALGEWFH | SGPPLASRYCY | RDRQFSS | QSSDNSDSFK |
| | SEQ ID | SEQ ID NO: 99 | YGMDV | SEQ ID | SEQ ID | M |
| | NO: 85 | | SEQ ID NO: 100 | NO: 101 | NO: 102 | SEQ ID |
| | | | | | | NO: 107 |
| 27 | DYAMA | FMRGWAYGGSAQFAAF | EQRNKDYRYGQEGFGY | RASHFIANYVN | ESSTLQR | QQSHSPPVT |
| | SEQ ID | AVG | SYGMDV | SEQ ID | SEQ ID | SEQ ID |
| | NO: 108 | SEQ ID NO: 109 | SEQ ID NO: 110 | NO: 111 | NO: 112 | NO: 113 |
| 28 | DYAMA | FIRGWAYGQAAQYGKS | EQRGGDGRYSGDGFGY | RASHFIANYVN | QSWTLNR | QQSHSPPLS |
| | SEQ ID | ASG | PYGMDV | SEQ ID | SEQ ID | SEQ ID |
| | NO: 108 | SEQ ID NO: 114 | SEQ ID NO: 115 | NO: 111 | NO: 116 | NO: 117 |
| 29 | DYAMA | FIRGWAYGQSAQYGKS | EQRGANGRYGGDGFGY | RASHFIANYVN | ESSTLNR | QQSHSPPVS |
| | SEQ ID | ASG | SYGMDV | SEQ ID | SEQ ID | SEQ ID |
| | NO: 108 | SEQ ID NO: 118 | SEQ ID NO: 119 | NO: 111 | NO: 120 | NO: 121 |

TABLE A2
CDRs (Chothia) for Anti-HIV gp120 V3 Glycan-Directed Antibodies or Antigen-Binding
Fragments Thereof

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 30 | GASISD | YVHKSGDTN | TLHGRRIYGIVAFNEWF | GEKSLGSRAVQ | NNQDRP | HIWDSRVPTK |
| | SEQ ID | SEQ ID NO: 124 | TYFYMDV | SEQ ID NO: 10 | S | WV |
| | NO: 123 | | SEQ ID NO: 9 | | SEQ ID | SEQ ID |
| | | | | | NO: 11 | NO: 12 |
| 31 | GDSMNN | YISDRESAT | ARRGQRIYGVVSFGEFF | GRQALGSRAVQ | NNQDRP | HMWDSRSGFS |
| | SEQ ID | SEQ ID NO: 126 | YYYSMDV | SEQ ID NO: 17 | S | WS |
| | NO: 125 | | SEQ ID NO: 16 | | SEQ ID | SEQ ID |
| | | | | | NO: 11 | NO: 18 |
| 32 | GGSISN | YISDRETTT | ARRGQRIYGVVSFGEFF | GRQALGSRAVQ | NNQDRP | HMWDSRSGFS |
| | SEQ ID | SEQ ID NO: 128 | YYYYMDV | SEQ ID NO:17 | S | WS |
| | NO: 127 | | SEQ ID NO: 20 | | SEQ ID | SEQ ID |
| | | | | | NO: 11 | NO: 18 |
| 33 | NGSVSG | YFSDTDRSE | AQQGKRIYGIVSFGEFF | GERSRGSRAVQ | NNQDRP | HYWDSRSPIS |
| | SEQ ID | SEQ ID NO: 130 | YYYYMDA | SEQ ID NO: 24 | A | WI |
| | NO: 129 | | SEQ ID NO: 23 | | SEQ ID | SEQ ID |
| | | | | | NO: 25 | NO: 26 |
| 34 | NGSVSG | YFSDTDRSE | AQQGKRIYGIVSFGELF | GERSRGSRAVQ | NNQDRP | HYWDSRSPIS |
| | SEQ ID | SEQ ID NO: 130 | YYYYMDA | SEQ ID NO: 24 | A | WI |
| | NO: 129 | | SEQ ID NO: 27 | | SEQ ID | SEQ ID |
| | | | | | NO: 25 | NO: 26 |
| 35 | GTLVRD | YVHDSGDTN | TKHGRRIYGVVAFKEWF | GEESLGSRSVI | NNNDRP | HIWDSRRPTN |
| | SEQ ID | SEQ ID NO: 132 | TYFYMDV | SEQ ID NO: 31 | S | WV |
| | NO: 131 | | SEQ ID NO: 30 | | SEQ ID | SEQ ID |
| | | | | | NO: 32 | NO: 33 |
| 36 | GASIND | YVHHSGDTN | ALHGKRIYGIVALGELF | GKESIGSRAVQ | NNQDRP | HIYDARGGTN |
| | SEQ ID | SEQ ID NO: 134 | TYFYMDV | SEQ ID NO: 37 | A | WV |
| | NO: 133 | | SEQ ID NO: 36 | | SEQ ID | SEQ ID |
| | | | | | NO: 25 | NO: 38 |
| 37 | GESTGACT | SLSHCQSFWGSGW | FDGEVLVYNHWPKPAWV | NGTATNFVS | GVDKRP | GSLVGNWDVI |
| | SEQ ID | TF | DL | SEQ ID NO: 42 | P | SEQ ID |
| | NO: 135 | SEQ ID NO: 136 | SEQ ID NO: 41 | | SEQ ID | NO: 44 |
| | | | | | NO: 43 | |
| 38 | GDSTAACD | GLSHCAGYYNTGW | FDGEVLVYHDWPKPAWV | TGTSNRFVS | GVNKRP | SSLVGNWDVI |
| | SEQ ID | TY | DL | SEQ ID NO: 48 | S | SEQ ID |
| | NO: 137 | SEQ ID NO: 138 | SEQ ID NO: 47 | | SEQ ID | NO: 50 |
| | | | | | NO: 49 | |
| 39 | GDSTAACD | SLSHCAGYYNSGW | FGGDVLVYHDWPKPAWV | TGNINNFVS | GVNKRP | GSLAGNWDVV |
| | SEQ ID | TY | DL | SEQ ID NO: 53 | S | SEQ ID |
| | NO: 137 | SEQ ID NO: 106 | SEQ ID NO: 52 | | SEQ ID | NO: 54 |
| | | | | | NO: 49 | |
| 40 | GDSTAACN | SLSHCASYWNRGW | FGGEVLRYTDWPKPAWV | TGTSNNFVS | DVNKRP | GSLVGNWDVI |
| | SEQ ID | TYHNPSLKS | DL | SEQ ID NO: 58 | S | SEQ ID |
| | NO: 139 | SEQ ID NO: 56 | SEQ ID NO: 57 | | SEQ ID | NO: 44 |
| | | | | | NO: 59 | |
| 41 | GDSTAGCD | GLSHCAGYYNTGW | FDGEVLVYNDWPKPAWV | TGTSNNFVS | GVNKRP | GSLVGNWDVI |
| | SEQ ID | TY | DL | SEQ ID NO: 58 | S | SEQ ID |
| | NO: 141 | SEQ ID NO: 138 | SEQ ID NO: 63 | | SEQ ID | NO: 44 |
| | | | | | NO: 49 | |
| 42 | GESINTGH | HIHYTTAVL | SGGDILYYYEWQKPHWF | NGTSSDIGGWNF | EVNKRP | SSLFGRWDVV |
| | SEQ ID | SEQ ID NO: 143 | SP | VS | S | SEQ ID |
| | NO: 142 | | SEQ ID NO: 66 | SEQ ID NO: 67 | SEQ ID | NO: 69 |
| | | | | | NO: 68 | |
| 43 | GGSMRGTDWGEN | SIHWRGRTTH | HKYHDIFRVVPVAGWFD | RASQNVKNNLA | DASSRA | QQYEEWPRT |
| | D | SEQ ID NO: 145 | P | SEQ ID NO: 73 | G | SEQ ID |
| | SEQ ID | | SEQ ID NO: 72 | | SEQ ID | NO: 75 |
| | NO: 144 | | | | NO: 74 | |
| 44 | GGSIRGGEWGDS | SIHWRGTTH | HKYHDIVMVVPIAGWFD | RASQSVKNNLA | DTSSRA | QQYEEWPRT |
| | D | SEQ ID NO: 147 | P | SEQ ID NO: 79 | S | SEQ ID |
| | SEQ ID | | SEQ ID NO: 78 | | SEQ ID | NO: 75 |
| | NO: 146 | | | | NO: 80 | |
| 45 | GDSIRGGEWGDK | SIHWRGTTH | HRHHDVFMLVPIAGWFD | RASQNINKNLA | ETYSKI | QQYEEWPRT |
| | D | SEQ ID NO: 147 | V | SEQ ID NO: 84 | A | SEQ ID |
| | SEQ ID | | SEQ ID NO: 83 | | SEQ ID | NO: 75 |
| | NO: 148 | | | | NO: 62 | |
| 46 | QDSRPSDH | HYNGA | NAIRIYGVVALGEWFHY | SGAPLTSRFTY | RSSQRS | QSSDTSDSYK |
| | SEQ ID | SEQ ID NO: 150 | GMDV | SEQ ID NO: 88 | S | M |
| | NO: 149 | | SEQ ID NO: 87 | | SEQ ID | SEQ ID |
| | | | | | NO: 89 | NO: 90 |
| 47 | NDSRPSDH | HYNGA | NAIRIYGVVALGEWFHY | SGAPLTSRFTY | RSSQRS | QSSDTSDSYK |
| | SEQ ID | SEQ ID NO: 150 | GMDV | SEQ ID NO: 88 | S | M |
| | NO: 151 | | SEQ ID NO: 87 | | SEQ ID | SEQ ID |
| | | | | | NO: 89 | NO: 90 |
| 48 | GDSRPSDH | HYNGD | NVIRVFGVISLGEWFHY | SGPPLASRYTY | RDRQFP | QSSDTSDSYK |
| | SEQ ID | SEQ ID NO: 153 | GMDV | SEQ ID NO: 93 | S | M |
| | NO: 152 | | SEQ ID NO: 92 | | SEQ ID | SEQ ID |
| | | | | | NO: 94 | NO: 90 |
| 49 | NDSRPSDH | HYNGA | NAIRIYGVVALGEWFHY | SGAALTSRFTY | RTSQRS | QSSDTSDSYK |
| | SEQ ID | SEQ ID NO: 150 | GMDV | SEQ ID NO: 97 | S | M |
| | NO: 151 | | SEQ ID NO: 87 | | SEQ ID | SEQ ID |
| | | | | | NO: 98 | NO: 90 |
| 50 | GDSRPSDH | HYGGD | NVIRVFGVIALGEWFHY | SGPPLASRYCY | RDRQFS | QSSDINDSYK |
| | SEQ ID | SEQ ID NO: 122 | GMDV | SEQ ID | S | M |
| | NO: 152 | | SEQ ID NO: 100 | NO: 101 | SEQ ID | SEQ ID |
| | | | | | NO: 102 | NO: 95 |
| 51 | GDSRPSDH | HYGGD | NVIRVFGVIALGEWFHY | SGPPLASRYCY | RDRQFS | QSSDTSDSFK |
| | SEQ ID | SEQ ID NO: 122 | GMDV | SEQ ID | S | M |
| | NO: 152 | | SEQ ID NO: 100 | NO: 101 | SEQ ID | SEQ ID |
| | | | | | NO: 102 | NO: 103 |
| 52 | GDSRPSDH | HYGGD | NVIRVFGVIALGEWFHY | SGPPLATRYCY | RDRQFS | QSSDTSDSYK |
| | SEQ ID | SEQ ID NO: 122 | GMDV | SEQ ID | S | M |
| | NO: 152 | | SEQ ID NO: 100 | NO: 104 | SEQ ID | SEQ ID |
| | | | | | NO: 102 | NO: 90 |
| 53 | GDSRPSDH | HYGGD | NVIRVFGVIALGEWFHY | SGPPLASRYCY | RDRQFS | QSSDNSDSFK |
| | SEQ ID | SEQ ID NO: 122 | GMDV | SEQ ID | S | M |
| | NO: 152 | | SEQ ID NO: 100 | NO: 101 | SEQ ID | SEQ ID |
| | | | | | NO: 102 | NO: 107 |
| 54 | GFYFPDY | RGWAYGGS | EQRNKDYRYGQEGFGYS | RASHFIANYVN | ESSTLQ | QQSHSPPVT |
| | SEQ ID | SEQ ID NO: 155 | YGMDV | SEQ ID | R | SEQ ID |
| | NO: 154 | | SEQ ID NO: 110 | NO: 111 | SEQ ID | NO: 113 |
| | | | | | NO: 112 | |
| 55 | DFYFPDY | RGWAYGQA | EQRGGDGRYSGDGFGYP | RASHFIANYVN | QSWTLN | QQSHSPPLS |
| | SEQ ID | SEQ ID NO: 157 | YGMDV | SEQ ID | R | SEQ ID |
| | NO: 156 | | SEQ ID NO: 115 | NO: 111 | SEQ ID | NO: 117 |
| | | | | | NO: 116 | |
| 56 | DFYFPDY | RGWAYGQS | EQRGANGRYGGDGFGYS | RASHFIANYVN | ESSTLN | QQSHSPPVS |
| | SEQ ID | SEQ ID NO: 159 | YGMDV | SEQ ID | R | SEQ ID |
| | NO: 158 | | SEQ ID NO: 119 | NO: 111 | SEQ ID | NO: 121 |
| | | | | | NO: 120 | |

TABLE A3
CDRs (IMGT) for Anti-HIV gp120 V3 Glycan-Directed Antibodies or Antigen-Binding Fragments
Thereof

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 57 | GASISDSY | VHKSGDT | ARTLHGRRIYGIVAFNEWFTYFYM | SLGSRA | NNQ | HIWDSRVPTKW |
| | SEQ ID | SEQ ID | DV | SEQ ID | SEQ ID | V |
| | NO: 160 | NO: 161 | SEQ ID NO: 162 | NO: 163 | NO: 164 | SEQ ID |
| | | | | | | NO: 12 |
| 58 | GDSMNNYY | ISDRESA | ATARRGQRIYGVVSFGEFFYYYSM | ALGSRA | NNQ | HMWDSRSGFSW |
| | SEQ ID | SEQ ID | DV | SEQ ID | SEQ ID | S |
| | NO: 165 | NO: 166 | SEQ ID NO: 167 | NO: 168 | NO: 164 | SEQ ID |
| | | | | | | NO: 18 |
| 180 | GDSMNNYY | ISDRESA | ARARRGQRIYGVVSFGEFFYYYSM | ALGSRA | NNQ | HMWDSRSGFSW |
| | SEQ ID | SEQ ID | DV | SEQ ID | SEQ ID | S |
| | NO: 165 | NO: 166 | SEQ ID NO: 461 | NO: 168 | NO: 164 | SEQ ID |
| | | | | | | NO: 18 |
| 59 | GGSISNYY | ISDRETT | ATARRGQRIYGVVSFGEFFYYYYM | ALGSRA | NNQ | HMWDSRSGFSW |
| | SEQ ID | SEQ ID | DV | SEQ ID | SEQ ID | S |
| | NO: 169 | NO: 170 | SEQ ID NO: 171 | NO: 168 | NO: 164 | SEQ ID |
| | | | | | | NO: 18 |
| 60 | NGSVSGRF | FSDTDRS | ARAQQGKRIYGIVSFGELFYYYYM | SRGSRA | NNQ | HYWDSRSPISW |
| | SEQ ID | SEQ ID | DA | SEQ ID | SEQ ID | I |
| | NO: 172 | NO: 173 | SEQ ID NO: 174 | NO: 175 | NO: 164 | SEQ ID |
| | | | | | | NO: 26 |
| 61 | NGSVSGRF | FSDTDRS | ARAQQGKRIYGIVSFGEFFYYYYM | SRGSRA | NNQ | HYWDSRSPISW |
| | SEQ ID | SEQ ID | DA | SEQ ID | SEQ ID | I |
| | NO: 172 | NO: 173 | SEQ ID NO: 176 | NO: 175 | NO: 164 | SEQ ID |
| | | | | | | NO: 26 |
| 62 | GTLVRDNY | VHDSGDT | ATTKHGRRIYGVVAFKEWFTYFYM | SIGSRA | NNQ | HIYDARGGTNW |
| | SEQ ID | SEQ ID | DV | SEQ ID | SEQ ID | V |
| | NO: 177 | NO: 178 | SEQ ID NO: 179 | NO: 180 | NO: 164 | SEQ ID |
| | | | | | | NO: 38 |
| 63 | GASINDAY | VHHSGDT | ARALHGKRIYGIVALGELFTYFYM | SLGSRS | NNN | HIWDSRRPTNW |
| | SEQ ID | SEQ ID | DV | SEQ ID | SEQ ID | V |
| | NO: 181 | NO: 182 | SEQ ID NO: 183 | NO: 184 | NO: 185 | SEQ ID |
| | | | | | | NO: 33 |
| 64 | GESTGACTY | LSHCQSFWGSGW | ARFDGEVLVYNHWPKPAWVDL | ATNF | GVD | GSLVGNWDVI |
| | F | T | SEQ ID NO: 188 | SEQ ID | SEQ ID | SEQ ID |
| | SEQ ID | SEQ ID | | NO: 189 | NO: 190 | NO: 44 |
| | NO: 186 | NO: 187 | | | | |
| 65 | GDSTAACDY | LSHCAGYYNTGW | ARFDGEVLVYHDWPKPAWVDL | SNRF | GVN | SSLVGNWDVI |
| | F | T | SEQ ID NO: 193 | SEQ ID | SEQ ID | SEQ ID |
| | SEQ ID | SEQ ID | | NO: 194 | NO: 195 | NO: 50 |
| | NO: 191 | NO: 192 | | | | |
| 66 | GDSTAACDY | LSHCAGYYNSGW | ARFGGDVLVYHDWPKPAWVDL | INNF | GVN | GSLAGNWDVV |
| | F | T | SEQ ID NO: 197 | SEQ ID | SEQ ID | SEQ ID |
| | SEQ ID | SEQ ID | | NO: 198 | NO: 195 | NO: 54 |
| | NO: 191 | NO: 196 | | | | |
| 67 | GDSTAACNS | LSHCASYWNRGW | ARFGGEVLRYTDWPKPAWVDL | SNNF | DVN | GSLVGNWDVI |
| | F | T | SEQ ID NO: 201 | SEQ ID | SEQ ID | SEQ ID |
| | SEQ ID | SEQ ID | | NO: 202 | NO: 399 | NO: 44 |
| | NO: 199 | NO: 200 | | | | |
| 68 | GDSTAGCDY | LSHCAGYYNTGW | ARFDGEVLVYNDWPKPAWVDL | SNNF | GVN | GSLVGNWDVI |
| | F | T | SEQ ID NO: 205 | SEQ ID | SEQ ID | SEQ ID |
| | SEQ ID | SEQ ID | | NO: 202 | NO: 195 | NO: 44 |
| | NO: 203 | NO: 204 | | | | |
| 69 | GESINTGHY | IHYTTAV | VRSGGDILYYYEWQKPHWFSP | SSDIGGWNF | EVN | SSLFGRWDVV |
| | Y | SEQ ID | SEQ ID NO: 208 | SEQ ID | SEQ ID | SEQ ID |
| | SEQ ID | NO: 207 | | NO: 209 | NO: 210 | NO: 69 |
| | NO: 206 | | | | | |
| 70 | GGSMRGTDW | IHWRGRTT | ARHKYHDIFRVVPVAGWFDP | QNVKNN | DAS | QQYEEWPRT |
| | GENDFH | SEQ ID | SEQ ID NO: 213 | SEQ ID | SEQ ID | SEQ ID |
| | SEQ ID | NO: 212 | | NO: 214 | NO: 215 | NO: 75 |
| | NO: 211 | | | | | |
| 71 | GGSIRGGEW | IHWRGTT | VKHKYHDIVMVVPIAGWFDP | QSVKNN | DTS | QQYEEWPRT |
| | GDSDYH | SEQ ID | SEQ ID NO: 218 | SEQ ID | SEQ ID | SEQ ID |
| | SEQ ID | NO: 217 | | NO: 219 | NO: 220 | NO: 75 |
| | NO: 216 | | | | | |
| 72 | GDSIRGGEW | IHWRGTT | ARHRHHDVFMLVPIAGWFDV | QNINKN | ETY | QQYEEWPRT |
| | GDKDYH | SEQ ID | SEQ ID NO: 60 | SEQ ID | SEQ ID | SEQ ID |
| | SEQ ID | NO: 217 | | NO: 223 | NO: 224 | NO: 75 |
| | NO: 221 | | | | | |
| 73 | QDSRPSDHS | IHYNGAT | NAIRIYGVVALGEWFHYGMDV | PLTSRF | RSS | QSSDTSDSYKM |
| | SEQ ID | SEQ ID | SEQ ID NO: 87 | SEQ ID | SEQ ID | SEQ ID |
| | NO: 225 | NO: 226 | | NO: 227 | NO: 228 | NO: 90 |
| 74 | NDSRPSDHS | IHYNGAT | NAIRIYGVVALGEWFHYGMDV | PLTSRF | RSS | QSSDTSDSYKM |
| | SEQ ID | SEQ ID | SEQ ID NO: 87 | SEQ ID | SEQ ID | SEQ ID |
| | NO: 229 | NO: 226 | | NO: 227 | NO: 228 | NO: 90 |
| 75 | GDSRPSDHS | VHYNGDN | NVIRVFGVISLGEWFHYGMDV | PLASRY | RDR | QSSDTSDSYKM |
| | SEQ ID | SEQ ID | SEQ ID NO: 92 | SEQ ID | SEQ ID | SEQ ID |
| | NO: 230 | NO: 231 | | NO: 232 | NO: 233 | NO: 90 |
| 76 | GDSRPSDHS | VHYNGDT | NVIRVFGVISLGEWFHYGMDV | PLASRY | RDR | QSSDTSDSYKM |
| | SEQ ID | SEQ ID | SEQ ID NO: 92 | SEQ ID | SEQ ID | SEQ ID |
| | NO: 230 | NO: 234 | | NO: 232 | NO: 233 | NO: 90 |
| 77 | NDSRPSDHS | IHYNGAT | NAIRIYGVVALGEWFHYGMDV | ALTSRF | RTS | QSSDTSDSYKM |
| | SEQ ID | SEQ ID | SEQ ID NO: 87 | SEQ ID | SEQ ID | SEQ ID |
| | NO: 229 | NO: 226 | | NO: 235 | NO: 398 | NO: 90 |
| 78 | GDSRPSDHS | IHYGGDI | NVIRVFGVIALGEWFHYGMDV | PLASRY | RDR | QSSDINDSYKM |
| | SEQ ID | SEQ ID | SEQ ID NO: 100 | SEQ ID | SEQ ID | SEQ ID |
| | NO: 230 | NO: 236 | | NO: 232 | NO: 233 | NO: 95 |
| 79 | GDSRPSDHS | IHYGGDI | NVIRVFGVIALGEWFHYGMDV | PLASRY | RDR | QSSDTSDSFKM |
| | SEQ ID | SEQ ID | SEQ ID NO: 100 | SEQ ID | SEQ ID | SEQ ID |
| | NO: 230 | NO: 236 | | NO: 232 | NO: 233 | NO: 103 |
| 80 | GDSRPSDHS | IHYGGDI | NVIRVFGVIALGEWFHYGMDV | PLATRY | RDR | QSSDTSDSYKM |
| | SEQ ID | SEQ ID | SEQ ID NO: 100 | SEQ ID | SEQ ID | SEQ ID |
| | NO: 230 | NO: 236 | | NO: 237 | NO: 233 | NO: 90 |
| 81 | GDSRPSDHS | IHYNGDK | NVIRVFGVISLGEWFHYGMDV | PLASRY | RDR | QSSDTSDSYKM |
| | SEQ ID | SEQ ID | SEQ ID NO: 92 | SEQ ID | SEQ ID | SEQ ID |
| | NO: 230 | NO: 238 | | NO: 232 | NO: 233 | NO: 90 |
| 82 | GDSRPSDHS | IHYGGDI | NVIRVFGVIALGEWFHYGMDV | PLASRY | RDR | QSSDNSDSFKM |
| | SEQ ID | SEQ ID | SEQ ID NO: 100 | SEQ ID | SEQ ID | SEQ ID |
| | NO: 230 | NO: 236 | | NO: 232 | NO: 233 | NO: 107 |
| 83 | GFYFPDYA | MRGWAYGGSA | EQRNKDYRYGQEGFGYSYGMDV | HFIANY | ESS | QQSHSPPVT |
| | SEQ ID | SEQ ID | SEQ ID NO: 110 | SEQ ID | SEQ ID | SEQ ID |
| | NO: 239 | NO: 240 | | NO: 241 | NO: 242 | NO: 113 |
| 84 | DFYFPDYA | IRGWAYGQAA | EQRGGDGRYSGDGFGYPYGMDV | HFIANY | QSW | QQSHSPPLS |
| | SEQ ID | SEQ ID | SEQ ID NO: 115 | SEQ ID | SEQ ID | SEQ ID |
| | NO: 243 | NO: 244 | | NO: 241 | NO: 245 | NO: 117 |
| 85 | DFYFPDYA | IRGWAYGQSA | EQRGANGRYGGDGFGYSYGMDV | HFIANY | ESS | QQSHSPPVS |
| | SEQ ID | SEQ ID | SEQ ID NO: 119 | SEQ ID | SEQ ID | SEQ ID |
| | NO: 243 | NO: 246 | | NO: 241 | NO: 247 | NO: 121 |

TABLE A4
CDRs (Honegger) for Anti-HIV gp120 V3 Glycan-Directed Antibodies or Antigen-Binding Fragments
Thereof

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 86 | VSGASISDSY | VHKSGDTNYSPSLKS | TLHGRRIYGIV | EKSLGSRA | NNQDRPSGIPER | WDSRVPTKW |
| | SEQ ID | R | AFNEWFTYFYM | SEQ ID | SEQ ID | SEQ ID |
| | NO: 248 | SEQ ID NO: 249 | D | NO: 251 | NO: 252 | NO: 253 |
| | | | SEQ ID | | | |
| | | | NO: 250 | | | |
| 87 | VSGASISDSY | VHKSGDTNYNPSLKS | TLHGRRIYGIV | EKSLGSRA | NNQDRPSGIPER | WDSRVPTKW |
| | SEQ ID | R | AFNEWFTYFYM | SEQ ID | SEQ ID | SEQ ID |
| | NO: 248 | SEQ ID NO: 254 | D | NO: 251 | NO: 252 | NO: 253 |
| | | | SEQ ID | | | |
| | | | NO: 250 | | | |
| 88 | VSGDSMNNYY | ISDRESATYNPSLNS | ARRGQRIYGVV | RQALGSRA | NNQDRPSGIPER | WDSRSGFSW |
| | SEQ ID | R | SFGEFFYYYSM | SEQ ID | SEQ ID | SEQ ID |
| | NO: 255 | SEQ ID NO: 256 | D | NO: 258 | NO: 252 | NO: 259 |
| | | | SEQ ID | | | |
| | | | NO: 257 | | | |
| 89 | VSGGSISNYY | ISDRETTTYNPSLNS | ARRGQRIYGVV | RQALGSRA | NNQDRPSGIPER | WDSRSGFSW |
| | SEQ ID | R | SFGEFFYYYYM | SEQ ID | SEQ ID | SEQ ID |
| | NO: 260 | SEQ ID NO: 261 | D | NO: 258 | NO: 252 | NO: 259 |
| | | | SEQ ID | | | |
| | | | NO: 262 | | | |
| 90 | VSNGSVSGRF | FSDTDRSEYNPSLRS | AQQGKRIYGIV | ERSRGSRA | NNQDRPAGVSER | WDSRSPISW |
| | SEQ ID | R | SFGELFYYYYM | SEQ ID | SEQ ID | SEQ ID |
| | NO: 263 | SEQ ID NO: 264 | D | NO: 266 | NO: 267 | NO: 268 |
| | | | SEQ ID | | | |
| | | | NO: 265 | | | |
| 91 | VSNGSVSGRF | FSDTDRSEYNPSLRS | AQQGKRIYGIV | ERSRGSRA | NNQDRPAGVSER | WDSRSPISW |
| | SEQ ID | R | SFGEFFYYYYM | SEQ ID | SEQ ID | SEQ ID |
| | NO: 263 | SEQ ID NO: 264 | D | NO: 266 | NO: 267 | NO: 268 |
| | | | SEQ ID | | | |
| | | | NO: 397 | | | |
| 92 | VSGASINDAY | VHHSGDTNYNPSLKR | ALHGKRIYGIV | KESIGSRA | NNQDRPAGVPER | YDARGGTNW |
| | SEQ ID | R | ALGELFTYFYM | SEQ ID | SEQ ID | SEQ ID |
| | NO: 269 | SEQ ID NO: 270 | D | NO: 272 | NO: 273 | NO: 274 |
| | | | SEQ ID | | | |
| | | | NO: 271 | | | |
| 93 | VSGTLVRDNY | VHDSGDTNYNPSLKS | TKHGRRIYGVV | EESLGSRS | NNNDRPSGIPDR | WDSRRPTNW |
| | SEQ ID | R | AFKEWFTYFYM | SEQ ID | SEQ ID | SEQ ID |
| | NO: 275 | SEQ ID NO: 276 | D | NO: 278 | NO: 279 | NO: 280 |
| | | | SEQ ID | | | |
| | | | NO: 277 | | | |
| 94 | VSGESTGACTYF | LSHCQSFWGSGWTFH | FDGEVLVYNHW | GTATNF | GVDKRPPGVPDR | LVGNWDV |
| | SEQ ID | NPSLKSR | PKPAWVD | SEQ ID | SEQ ID | SEQ ID |
| | NO: 281 | SEQ ID NO: 282 | SEQ ID | NO: 284 | NO: 285 | NO: 286 |
| | | | NO: 283 | | | |
| 95 | VSGDSTAACDYF | LSHCAGYYNTGWTYH | FDGEVLVYHDW | GTSNRF | GVNKRPSGVPDR | LVGNWDV |
| | SEQ ID | NPSLKSR | PKPAWVD | SEQ ID | SEQ ID | SEQ ID |
| | NO: 287 | SEQ ID NO: 288 | SEQ ID | NO: 290 | NO: 291 | NO: 286 |
| | | | NO: 289 | | | |
| 96 | VSGDSTAACDYF | LSHCAGYYNSGWTYH | FGGDVLVYHDW | GNINNF | GVNKRPSGVPDR | LAGNWDV |
| | SEQ ID | NPSLKSR | PKPAWVD | SEQ ID | SEQ ID | SEQ ID |
| | NO: 287 | SEQ ID NO: 292 | SEQ ID | NO: 294 | NO: 295 | NO: 296 |
| | | | NO: 293 | | | |
| 97 | VSGDSTAACNSF | LSHCASYWNRGWTYH | FGGEVLRYTDW | GTSNNF | DVNKRPSGVPDR | LVGNWDV |
| | SEQ ID | NPSLKSR | PKPAWVD | SEQ ID | SEQ ID | SEQ ID |
| | NO: 297 | SEQ ID NO: 298 | SEQ ID | NO: 300 | NO: 301 | NO: 286 |
| | | | NO: 299 | | | |
| 98 | VSGDSTAGCDYF | LSHCAGYYNTGWTYH | FDGEVLVYNDW | GTSNNF | GVNKRPSGVPDR | LVGNWDV |
| | SEQ ID | NPSLKSR | PKPAWVD | SEQ ID | SEQ ID | SEQ ID |
| | NO: 302 | SEQ ID NO: 288 | SEQ ID | NO: 300 | NO: 295 | NO: 286 |
| | | | NO: 303 | | | |
| 99 | VSGESINTGHYY | IHYTTAVLHNPSLKS | SGGDILYYYEW | GTSSDIGGWN | EVNKRPSGVPGR | LFGRWDV |
| | SEQ ID | R | QKPHWFS | F | SEQ ID | SEQ ID |
| | NO: 304 | SEQ ID NO: 305 | SEQ ID | SEQ ID | NO: 308 | NO: 309 |
| | | | NO: 306 | NO: 307 | | |
| 100 | VSGGSMRGTDWG | IHWRGRITHYKTSFR | HKYHDIFRVVP | ASQNVKNN | DASSRAGGIPDR | YEEWPR |
| | ENDFH | SR | VAGWFD | SEQ ID | SEQ ID | SEQ ID |
| | SEQ ID | SEQ ID NO: 311 | SEQ ID | NO: 313 | NO: 314 | NO: 315 |
| | NO: 310 | | NO: 312 | | | |
| 101 | ASGGSIRGGEWG | IHWRGTTHYNAPFRG | HKYHDIVMVVP | ASQSVKNN | DTSSRASGIPAR | YEEWPR |
| | DSDYH | R | IAGWFD | SEQ ID | SEQ ID | SEQ ID |
| | SEQ ID | SEQ ID NO: 317 | SEQ ID | NO: 319 | NO: 320 | NO: 315 |
| | NO: 316 | | NO: 318 | | | |
| 102 | VSGDSIRGGEWG | IHWRGTTHYKESLRR | HRHHDVFMLVP | ASQNINKN | ETYSKIAAFPAR | YEEWPR |
| | DKDYH | R | IAGWFD | SEQ ID | SEQ ID | SEQ ID |
| | SEQ ID | SEQ ID NO: 322 | SEQ ID | NO: 324 | NO: 325 | NO: 315 |
| | NO: 321 | | NO: 323 | | | |
| 103 | VSQDSRPSDHS | IHYNGATTYNPSLRS | NAIRIYGVVAL | GAPLTSRF | RSSQRSSGWSGR | SDTSDSYK |
| | SEQ ID | R | GEWFHYGMD | SEQ ID | SEQ ID | SEQ ID |
| | NO: 326 | SEQ ID NO: 327 | SEQ ID | NO: 329 | NO: 330 | NO: 331 |
| | | | NO: 328 | | | |
| 104 | VSNDSRPSDHS | IHYNGATTYNPSLRS | NAIRIYGVVAL | GAPLTSRF | RSSQRSSGWSGR | SDTSDSYK |
| | SEQ ID | R | GEWFHYGMD | SEQ ID | SEQ ID | SEQ ID |
| | NO: 332 | SEQ ID NO: 327 | SEQ ID | NO: 329 | NO: 330 | NO: 331 |
| | | | NO: 328 | | | |
| 105 | VFGDSRPSDHS | VHYNGDNTYNPSLRG | NVIRVFGVISL | GPPLASRY | RDRQFPSGVSGR | SDTSDSYK |
| | SEQ ID | R | GEWFHYGMD | SEQ ID | SEQ ID | SEQ ID |
| | NO: 333 | SEQ ID NO: 334 | SEQ ID | NO: 336 | NO: 337 | NO: 338 |
| | | | NO: 335 | | | |
| 106 | VFGDSRPSDHS | VHYNGDTTYNPSLRG | NVIRVFGVISL | GPPLASRY | RDRQFPSGVSGR | SDTSDSYK |
| | SEQ ID | R | GEWFHYGMD | SEQ ID | SEQ ID | SEQ ID |
| | NO: 333 | SEQ ID NO: 339 | SEQ ID | NO: 336 | NO: 337 | NO: 338 |
| | | | NO: 335 | | | |
| 107 | VSNDSRPSDHS | IHYNGATTYNPSLRS | NAIRIYGVVAL | GAALTSRF | RTSQRSSGWSGR | SDTSDSYK |
| | SEQ ID | R | GEWFHYGMD | SEQ ID | SEQ ID | SEQ ID |
| | NO: 332 | SEQ ID NO: 327 | SEQ ID | NO: 340 | NO: 341 | NO: 338 |
| | | | NO: 328 | | | |
| 108 | ISGDSRPSDHS | IHYGGDITYNPSLRS | NVIRVFGVIAL | GPPLASRY | RDRQFSSGMSGR | SDINDSYK |
| | SEQ ID | R | GEWFHYGMD | SEQ ID | SEQ ID | SEQ ID |
| | NO: 342 | SEQ ID NO: 343 | SEQ ID | NO: 336 | NO: 345 | NO: 346 |
| | | | NO: 344 | | | |
| 109 | ISGDSRPSDHS | IHYGGDITYNPSLRS | NVIRVFGVIAL | GPPLASRY | RDRQFSSGISGR | SDTSDSFK |
| | SEQ ID | R | GEWFHYGMD | SEQ ID | SEQ ID | SEQ ID |
| | NO: 342 | SEQ ID NO: 343 | SEQ ID | NO: 336 | NO: 347 | NO: 348 |
| | | | NO: 344 | | | |
| 110 | ISGDSRPSDHS | IHYGGDITYNPSLRS | NVIRVFGVIAL | GPPLATRY | RDRQFSSGVSGR | SDTSDSYK |
| | SEQ ID | R | GEWFHYGMD | SEQ ID | SEQ ID | SEQ ID |
| | NO: 342 | SEQ ID NO: 343 | SEQ ID | NO: 349 | NO: 350 | NO: 338 |
| | | | NO: 344 | | | |
| 111 | VFGDSRPSDHS | IHYNGDKTYNPSLRG | NVIRVFGVISL | GPPLASRY | RDRQFPSGVSGR | SDTSDSYK |
| | SEQ ID | R | GEWFHYGMD | SEQ ID | SEQ ID | SEQ ID |
| | NO: 333 | SEQ ID NO: 351 | SEQ ID | NO: 336 | NO: 337 | NO: 338 |
| | | | NO: 335 | | | |
| 112 | ISGDSRPSDHS | IHYGGDITYNPSLRS | NVIRVFGVIAL | GPPLASRY | RDRQFSSGISGR | SDNSDSFK |
| | SEQ ID | R | GEWFHYGMD | SEQ ID | SEQ ID | SEQ ID |
| | NO: 342 | SEQ ID NO: 343 | SEQ ID | NO: 336 | NO: 347 | NO: 352 |
| | | | NO: 344 | | | |
| 113 | ASGFYFPDYA | MRGWAYGGSAQFAAF | EQRNKDYRYGQ | ASHFIANY | ESSTLQRGVPSR | SHSPPV |
| | SEQ ID | AVGK | EGFGYSYGMD | SEQ ID | SEQ ID | SEQ ID |
| | NO: 353 | SEQ ID NO: 354 | SEQ ID | NO: 356 | NO: 357 | NO: 358 |
| | | | NO: 355 | | | |
| 114 | AFDFYFPDYA | IRGWAYGQAAQYGKS | EQRGGDGRYSG | ASHFIANY | QSWTLNRGIPSR | SHSPPL |
| | SEQ ID | ASGR | DGFGYPYGMD | SEQ ID | SEQ ID | SEQ ID |
| | NO: 359 | SEQ ID NO: 360 | SEQ ID | NO: 356 | NO: 362 | NO: 363 |
| | | | NO: 361 | | | |
| 115 | AFDFYFPDYA | IRGWAYGQSAQYGKS | EQRGANGRYGG | ASHFIANY | ESSTLNRGVPSR | SHSPPV |
| | SEQ ID | ASGR | DGFGYSYGMD | SEQ ID | SEQ ID | SEQ ID |
| | NO: 359 | SEQ ID NO: 364 | SEQ ID | NO: 356 | NO: 366 | NO: 358 |
| | | | NO: 365 | | | |

TABLE B
VH/VL for Anti-HIV gp120 V3 Glycan-Directed Antibodies or Antigen-Binding Fragments
Thereof

| | SEQ | | SEQ | |
|---|---|---|---|---|
| Ab | ID | | ID | |
| Name | NO | VH | NO | VL |
| 150 | 400 | QMQLQESGPGLVKPSETLSLTCSVSGASISDSYWSWI | 401 | SDISVAPGETARISCGEKSLGSRAVQWYQHRA |
| | | RRSPGKGLEWIGYVHKSGDTNYSPSLKSRVNLSLDTS | | GQAPSLIIYNNQDRPSGIPERFSGSPDSPFGT |
| | | KNQVSLSLVAATAADSGKYYCARTLHGRRIYGIVAFN | | TATLTITSVEAGDEADYYCHIWDSRVPTKWVF |
| | | EWFTYFYMDVWGNGTQVTVSS | | GGGTTLTVL |
| 151 | 402 | QMQLQESGPGLVKPSETLSLTCSVSGASISDSYWSWI | 403 | SDISVAPGETARISCGEKSLGSRAVQWYQHRA |
| | | RRSPGKGLEWIGYVHKSGDTNYNPSLKSRVHLSLDTS | | GQAPSLIIYNNQDRPSGIPERFSGSPDFRPGT |
| | | KNQVSLSLTGVTAADSGKYYCARTLHGRRIYGIVAFN | | TATLTITSVEAGDEADYYCHIWDSRVPTKWVF |
| | | EWFTYFYMDVWGTGTQVTVSS | | GGGTTLTVL |
| 181 | 462 | QMQLQESGPGLVKPSETLSLTCSVSGASISDSYWSWI | 463 | SDISVAPGETARISCGEKSLGSRAVQWYQHRA |
| | | RRSPGKGLEWIGYVHKSGDTNYNPSLKSRVHLSLDTS | | GQAPSLIIYNNQDRPSGIPERFSGSPDSRPGT |
| | | KNQVSLSLTGVTAADSGKYYCARTLHGRRIYGIVAFN | | TATLTITSVEAGDEADYYCHIWDSRVPTKWVF |
| | | EWFTYFYMDVWGTGTQVTVSS | | GGGTTLTVL |
| 182 | 464 | QMQLQESGPGLVKPSETLSLTCSVSGASISDSYWSWI | 465 | SDISVAPGETARISCGEKSLGSRAVQWYQQRA |
| | | RQPPGKGLEWIGYVHKSGDTNYSPSLKSRVNLSLDTS | | GQAPSLIIYNNQDRPSGIPERFSGSPDSGFGT |
| | | KNQVSLSLSAATAADSGVYYCARTLHGRRIYGIVAFN | | TATLTITSVEAGDEADYYCHIWDSRVPTKWVF |
| | | EWFTYFYMDVWGNGTQVTVSS | | GGGTTLTVL |
| 152 | 402 | QMQLQESGPGLVKPSETLSLTCSVSGASISDSYWSWI | 404 | SDISVAPGETARISCGEKSLGSRAVQWYQHRA |
| | | RRSPGKGLEWIGYVHKSGDTNYNPSLKSRVHLSLDTS | | GQAPSLIIYNNQDRPSGIPERFSGSPDSRPGT |
| | | KNQVSLSLTGVTAADSGKYYCARTLHGRRIYGIVAFN | | TATLTITSVEAGDEADYYCHIWDSRVPTKWVF |
| | | EWFTYFYMDVWGTGTQVTVSS | | GGGTTLTVL |
| 153 | 405 | QVQLQESGPGLVKPSETLSVTCSVSGDSMNNYYWTWI | 406 | SYVRPLSVALGETARISCGRQALGSRAVQWYQ |
| | | RQSPGKGLEWIGYISDRESATYNPSLNSRVVISRDTS | | HRPGQAPILLIYNNQDRPSGIPERFSGTPDIN |
| | | KNQLSLKLNSVTPADTAVYYCATARRGQRIYGVVSFG | | FGTRATLTISGVEAGDEADYYCHMWDSRSGFS |
| | | EFFYYYSMDVWGKGTTVTVSS | | WSFGGATRLTVL |
| 183 | 466 | QVQLQESGPGLVKPSETLSVTCSVSGDSMNNYYWTWI | 467 | SPVRPLSVALGETARISCGRQALGSRAVQWYQ |
| | | RQSPGKGLEWIGYISDRESATYNPSLNSRVTISRDTS | | HRPGQAPILLIYNNQDRPSGIPERFSGTPDIN |
| | | KNQFSLKLNSVTPADTAVYYCARARRGQRIYGVVSFG | | FGTRATLTISGVEAGDEADYYCHMWDSRSGFS |
| | | EFFYYYSMDVWGKGTTVTVSS | | WSFGGATRLTVL |
| 154 | 407 | QVQLQESGPGLVRPSETLSVICIVSGGSISNYYWTWI | 408 | SYVSPLSVALGETARISCGRQALGSRAVQWYQ |
| | | RQSPGKGLEWIGYISDRETTTYNPSLNSRAVISRDTS | | HKPGQAPILLIYNNQDRPSGIPERFSGTPDIN |
| | | KNQLSLQLRSVTTADTAIYFCATARRGQRIYGVVSFG | | FGTTATLTISGVEVGDEADYYCHMWDSRSGFS |
| | | EFFYYYYMDVWGKGTAVTVSS | | WSFGGATRLTV |
| 155 | 409 | QVHLQESGPGLVTPSETLSLICTVSNGSVSGRFWSWI | 410 | SLNPLSLAPGATAKIPCGERSRGSRAVQWYQQ |
| | | RQSPGRGLEWIGYFSDTDRSEYNPSLRSRLTLSVDRS | | KPGQAPTLIIYNNQDRPAGVSERFSGNPDVAI |
| | | KNQLSLRLKSVTAADSATYYCARAQQGKRIYGIVSFG | | GVTATLTISRVEVGDEADYYCHYWDSRSPISW |
| | | EFFYYYYMDAWGKGTPVTVSS | | IFGGGTQLTVL |
| 156 | 411 | QVHLQESGPGLVTPSETLSLICTVSNGSVSGRFWSWI | 412 | SLNPLSLAPGATAKIPCGERSRGSRAVQWYQQ |
| | | RQSPGRGLEWIGYFSDTDRSEYNPSLRSRLTLSVDRS | | KPGQAPTLIIYNNQDRPAGVSERFSGNPDVAI |
| | | KNQLSLKLKSVTAADSATYYCARAQQGKRIYGIVSFG | | GVTATLTISRVEVGDEGDYYCHYWDSRSPISW |
| | | ELFYYYYMDAWGKGTPVTVSS | | IFAGGTQLTVL |
| 157 | 413 | QVHLQESGPGLVKPSETLSLTCNVSGTLVRDNYWSWI | 414 | TFVSVAPGQTARITCGEESLGSRSVIWYQQRP |
| | | RQPLGKQPEWIGYVHDSGDTNYNPSLKSRVHLSLDKS | | GQAPSLIIYNNNDRPSGIPDRFSGSPGSTFGT |
| | | KNLVSLRLTGVTAADSAIYYCATTKHGRRIYGVVAFK | | TATLTITSVEAGDEADYYCHIWDSRRPTNWVF |
| | | EWFTYFYMDVWGKGTSVTVSS | | GEGTTLIVL |
| 158 | 415 | QLHLQESGPGLVKPPETLSLTCSVSGASINDAYWSWI | 416 | SSMSVSPGETAKISCGKESIGSRAVQWYQQKP |
| | | RQSPGKRPEWVGYVHHSGDTNYNPSLKRRVTFSLDTA | | GQPPSLIIYNNQDRPAGVPERFSASPDFRPGT |
| | | KNEVSLKLVDLTAADSATYFCARALHGKRIYGIVALG | | TATLTITNVDAEDEADYYCHIYDARGGTNWVF |
| | | ELFTYFYMDVWGKGTAVTVSS | | DRGTTLTVL |
| 159 | 417 | QSQLQESGPRLVEASETLSLTCNVSGESTGACTYFWG | 418 | QSALTQPPSASGSPGQSITISCNGTATNFVSW |
| | | WVRQAPGKGLEWIGSLSHCQSFWGSGWTFHNPSLKSR | | YQQFPDKAPKLIIFGVDKRPPGVPDRFSGSRS |
| | | LTISLDTPKNQVFLKLTSLTAADTATYYCARFDGEVL | | GTTASLTVSRLQTDDEAVYYCGSLVGNWDVIF |
| | | VYNHWPKPAWVDLWGRGIPVTVSS | | GGGTTLTVL |
| 160 | 419 | QPQLQESGPGLVEASETLSLTCTVSGDSTAACDYFWG | 420 | QSALTQPPSASGSPGQSISISCTGTSNRFVSW |
| | | WVRQPPGKGLEWIGGLSHCAGYYNTGWTYHNPSLKSR | | YQQHPGKAPKLVIYGVNKRPSGVPDRFSGSKS |
| | | LTISLDTPKNQVFLKLNSVTAADTAIYYCARFDGEVL | | GNTASLTVSGLQTDDEAVYYCSSLVGNWDVIF |
| | | VYHDWPKPAWVDLWGRGTLVTVSS | | GGGTKLTVL |
| 161 | 421 | QLQMQESGPGLVKPSETLSLSCTVSGDSIRGGEWGDK | 422 | EIVMTQSPDTLSVSPGETVTLSCRASQNINKN |
| | | DYHWGWVRHSAGKGLEWIGSIHWRGITHYKESLRRRV | | LAWYQYKPGQSPRLVIFETYSKIAAFPARFVA |
| | | SMSIDTSRNWFSLRLASVTAADTAVYFCARHRHHDVF | | SGSGTEFTLTINNMQSEDVAVYYCQQYEEWPR |
| | | MLVPIAGWFDVWGPGVQVTVSS | | TFGQGTKVDIK |
| 162 | 423 | QLQLQESGPGLVKPSETLSLICTVSGGSMRGTDWGEN | 424 | EIVMTQSPPTLSVSPGETATLSCRASQNVKNN |
| | | DFHYGWIRQSSAKGLEWIGSIHWRGRTTHYKTSFRSR | | LAWYQLKPGQAPRLLIFDASSRAGGIPDRFSG |
| | | AILSIDTSNNRFSLTFSFVTAADTAVYYCARHKYHDI | | SGYGTDFTLIVNSVQSEDFGDYFCQQYEEWPR |
| | | FRVVPVAGWFDPWGQGLLVTVSS | | TFGQGTKVDIK |
| 163 | 425 | EVHLEESGPGLVRPSETLSLICTASGGSIRGGEWGDS | 426 | EIMMTQSPAILSVSPGDRATLSCRASQSVKNN |
| | | DYHWGWVRHSPEKGLEWIGSIHWRGITHYNAPFRGRG | | LAWYQKRPGQAPRLLIFDTSSRASGIPARFSG |
| | | RLSIDLSRNQFSLRLTSVTAEDTAVYYCVKHKYHDIV | | GGSGTEFTLTVNSMQSEDFATYYCQQYEEWPR |
| | | MVVPIAGWFDPWGQGLQVTVSS | | TFGQGTKVEIK |
| 164 | 427 | QPQLQESGPGLVEASETLSLTCTVSGDSTAACDYFWG | 428 | QSALTQPPSASGSPGQSITISCTGNINNFVSW |
| | | WVRQPPGKGLEWIGSLSHCAGYYNSGWTYHNPSLKSR | | YQQHPGKAPKLVIYGVNKRPSGVPDRFSGSKS |
| | | LTISLDTPKNQVFLKLNSVTAADTAIYYCARFGGDVL | | GNAASLTVSGLQTDDEAVYYCGSLAGNWDVVF |
| | | VYHDWPKPAWVDLWGRGVLVTVSS | | GGGTKLTVL |
| 165 | 429 | QPQLQESGPTLVEASETLSLTCAVSGDSTAACNSFWG | 430 | QSALTQPPSASGSPGQSITISCTGTSNNFVSW |
| | | WVRQPPGKGLEWVGSLSHCASYWNRGWTYHNPSLKSR | | YQQHAGKAPKLVIYDVNKRPSGVPDRFSGSKS |
| | | LTLALDTPKNLVFLKLNSVTAADTATYYCARFGGEVL | | GNTASLTVSGLQTDDEAVYYCGSLVGNWDVIF |
| | | RYTDWPKPAWVDLWGRGTLVTVSS | | GGGTKLTVL |
| 166 | 431 | QPQLQESGPGLVEASETLSLTCTVSGDSTAGCDYFWG | 432 | QSALTQPPSASGSPGQSITISCTGTSNNFVSW |
| | | WVRQPPGKGLEWIGGLSHCAGYYNTGWTYHNPSLKSR | | YQQHPAKAPKLVIYGVNKRPSGVPDRFSGSKS |
| | | LTISLDTPKNQVFLKLNSVTAADTAIYYCARFDGEVL | | GNTASLTVSGLQTDDEAVYYCGSLVGNWDVIF |
| | | VYNDWPKPAWVDLWGRGTLVTVSS | | GGGTKLTVL |
| 167 | 433 | QVQLQESGPGLVKPAETLSLTCSVSGESINTGHYYWG | 434 | QSALTQPPSASGSLGQSVTISCNGTSSDIGGW |
| | | WVRQVPGKGLEWIGHIHYTTAVLHNPSLKSRLTIKIY | | NFVSWYQQFPGRAPRLIIFEVNKRPSGVPGRF |
| | | TLRNQITLRLSNVTAADTAVYHCVRSGGDILYYYEWQ | | SGSKSGNSASLTVSGLQSDDEGQYFCSSLFGR |
| | | KPHWFSPWGPGIHVTVSS | | WDVVFGGGTKLTVL |
| 168 | 435 | QVQLRESGPGLVKPSETLSLSCTVSQDSRPSDHSWTW | 436 | WASSELTQPPSVSVSPGQTARITCSGAPLTSR |
| | | VRQSPGKALEWIGDIHYNGATTYNPSLRSRVRIELDQ | | FTYWYRQKPGQAPVLIISRSSQRSSGWSGRFS |
| | | SIPRFSLKMTSMTAADTGMYYCARNAIRIYGVVALGE | | ASWSGTTVTLTIRGVQADDEADYYCQSSDTSD |
| | | WFHYGMDVWGQGTAVTVSS | | SYKMFGGGTKLTVL |
| 169 | 437 | QVQLRESGPGLVKPSETLSLSCTVSNDSRPSDHSWTW | 438 | SSELTQPPSVSVSPGQTARITCSGAPLTSRFT |
| | | VRQSPGKALEWIGDIHYNGATTYNPSLRSRVRIELDQ | | YWYRQKPGQAPVLIISRSSQRSSGWSGRFSAS |
| | | SIPRFSLKMTSMTAADTGMYYCARNAIRIYGVVALGE | | WSGTTVTLTIRGVQADDEADYYCQSSDTSDSY |
| | | WFHYGMDVWGQGTAVTVSS | | KMFGGGTKLTVL |
| 170 | 439 | EVQLRESGPRLVKPSETLSLSCDVFGDSRPSDHSWTW | 440 | SSELTQAPSVSVSPGQTATIACSGPPLASRYT |
| | | VRQPPGKALEWIGDVHYNGDNTYNPSLRGRVKIDVDR | | YWYRQKPGQAPVLIIFRDRQFPSGVSGRFSAS |
| | | STHRFSLTLKSLTAADTGIYFCARNVIRVFGVISLGE | | KSGTTATLTIRDVQVEDEGDYYCQSSDTSDSY |
| | | WFHYGMDVWGPGTAVIVSS | | KMFGGGTTLTVL |
| 171 | 441 | EVQLRESGPGLVKPSETLSLSCDVFGDSRPSDHSWTW | 442 | SSELTQAPSVSVSPGQTATIACSGPPLASRYT |
| | | VRQPPGKALEWIGDVHYNGDTTYNPSLRGRVKIDVDR | | YWYRQKPGQAPVLIIFRDRQFPSGVSGRFSAS |
| | | STHRFSLTLNSLTAADTGIYFCARNVIRVFGVISLGE | | KSGTTATLTIRDVQVEDEGDYYCQSSDTSDSY |
| | | WFHYGMDVWGQGTAVTVSS | | KMFGGGTTLTVL |
| 172 | 443 | QVQLRESGPGLVKPSETLSLICTVSNDSRPSDHSWTW | 444 | SSELTQPPSVSVSPGQTAKITCSGAALTSRFT |
| | | VRQSPGKALEWIGDIHYNGATTYNPSLRSRVRIELDQ | | YWYRQKPGQAPVLIISRTSQRSSGWSGRFSAS |
| | | SIPRFSLKMTSMTAADTGMYYCARNAIRIYGVVALGE | | WSGTTVTLTIRGVQADDEGDYYCQSSDTSDSY |
| | | WFHYGMDVWGQGTAVTVSS | | KMFGGGTKLTVL |
| 173 | 445 | EVQLRESGPGLVKPSGNMALTCTISGDSRPSDHSWTW | 446 | SSELTQTPSVTVSPGETARIACSGPPLASRYC |
| | | VRQSPGKALEWIGDIHYGGDITYNPSLRSRVKLEVDT | | YWYRQKPGQAPVLIIFRDRQFSSGMSGRFASS |
| | | STNRFFLKMTSLTVADTGIYFCARNVIRVFGVIALGE | | HSGTTVTLTIRDVRVEDEADYYCQSSDINDSY |
| | | WFHYGMDVWGQGTAITVSP | | KMFGGGTKVTVL |
| 174 | 447 | EVQLRESGPGLVKPSGNMALTCTISGDSRPSDHSWTW | 448 | SSELTQTASVTVSPGETARIACSGPPLASRYC |
| | | VRQSPGKTLEWIGDIHYGGDITYNPSLRSRVKLEVDT | | YWYRQKPGQAPVLIIFRDRQFSSGISGRFSSS |
| | | SSNRFFLKMTSLTVADTGIYFCARNVIRVFGVIALGE | | QSGTTVTLTIRDVRVEDEADYYCQSSDTSDSF |
| | | WFHYGMDVWGQGTAITVSP | | KMFGGGTKLTVL |
| 175 | 449 | QVQLRESGPGLVKPSGNMALTCTISGDSRPSDHSWTW | 450 | SSELTQAPSVTVSPGDTARIACSGPPLATRYC |
| | | VRQSPGKALEWIGDIHYGGDITYNPSLRSRVELEVDR | | YWYRQKSGQAPVLIIFRDRQFSSGVSGRFSSS |
| | | STNRFFLKMTSLSVADTGMYFCARNVIRVFGVIALGE | | QSGSTVTLTIRDVRVEDEADYYCQSSDTSDSY |
| | | WFHYGMDVWGQGTAITVSP | | KMFGGGTKLTVL |
| 176 | 451 | QVQLRESGPGLVKPSETLSLSCDVFGDSRPSDHSWTW | 452 | SSELTQAPSVSVSPGQTARIACSGPPLASRYT |
| | | VRQPPGKALEWIGDIHYNGDKTYNPSLRGRVKIDVDR | | YWYRQKPGQAPVLIIFRDRQFPSGVSGRFSAS |
| | | STHRFSLTLNSLTAADTGMYFCARNVIRVFGVISLGE | | KSGTTGILTIRDVQAEDEGDYYCQSSDTSDSY |
| | | WFHYGMDVWGPGTAVTV | | KMFGGGTTLTVL |
| 177 | 453 | QVQLRESGPGLVKPSGNMALTCTISGDSRPSDHSWTW | 454 | SSELTQAPSVTLSPGETARIACSGPPLASRYC |
| | | VRQSPGKALEWIGDIHYGGDITYNPSLRSRVKLEVDT | | YWYRQKPGQAPVLIIFRDRQFSSGISGRFSSS |
| | | SSNRFFLKMTSLTVADTGIYFCARNVIRVFGVIALGE | | QSGTTVTLTIRDVRVEDEADYYCQSSDNSDSF |
| | | WFHYGMDVWGQGTAITVSP | | KMFGGGTKLTVL |
| 178 | 455 | AEQLVESGGGLVPPGRSLRLSCSASGFYFPDYAMAWV | 456 | DIHMTQSPVSLSASVGDRVTITCRASHFIANY |
| | | RQAPGQGLQWVGFMRGWAYGGSAQFAAFAVGKFAISR | | VNWYQQKPGKAPTLLIFESSTLQRGVPSRFSA |
| | | DDGRNVVYLDVKNPTFEDTGVYFCAREQRNKDYRYGQ | | YGDGTEFTLSINTLQPEDFASYICQQSHSPPV |
| | | EGFGYSYGMDVWGRGTTVVVST | | TFGAGTRVDQK |
| 179 | 457 | EERLVESGGGLVPPGRSLRLSCSAFDFYFPDYAMAWV | 458 | DILMTQSPVSLSASIGERITITCRASHFIANY |
| | | RQAPGKGLEWIGFIRGWAYGQAAQYGKSASGRMTISR | | VNWYQQRPGKAPKLLIFQSWTLNRGIPSRFSG |
| | | DDSRRVVYLDIKSPIEEDTGAYFCAREQRGGDGRYSG | | YGDGTEFTLSISALQSEDFGTYICQQSHSPPL |
| | | DGFGYPYGMDVWGRGTMVTVSA | | SFGGGTRVDQT |
| 180 | 459 | EERLVESGGGLVPPGRSLRLSCSAFDFYFPDYAMAWV | 460 | DIQMTQSPFTLSASVGERVTITCRASHFIANY |
| | | RQAPGRALEWIGFIRGWAYGQSAQYGKSASGRMTISR | | VNWYQQRPGRAPKLLIFESSTLNRGVPSRFSG |
| | | DDSRRVVYLDIKSPTHEDTGVYFCAREQRGANGRYGG | | SGDGTEFTLSISALQSEDFATYICQQSHSPPV |
| | | DGFGYSYGMDVWGRGTMVSVSA | | SFGGGTRVDQT |

In some embodiments, the anti-HIV gp120 V3 glycan-directed antibody or antigen-binding fragment thereof comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 7, 8, 9, 10, 11 and 12; SEQ ID NOs.: 7, 13, 9, 10, 11 and 12; SEQ ID NOs.: 14, 15, 16, 17, 11 and 18; SEQ ID NOs.: 14, 19, 20, 17, 11 and 18; SEQ ID NOs.: 21, 22, 23, 24, 25 and 26; SEQ ID NOs.: 21, 22, 27, 24, 25 and 26; SEQ ID NOs.: 28, 29, 30, 31, 32 and 33; SEQ ID NOs.: 34, 35, 36, 37, 25 and 38; SEQ ID NOs.: 39, 40, 41, 42, 43 and 44; SEQ ID NOs.: 45, 46, 47, 48, 49 and 50; SEQ ID NOs.: 45, 51, 52, 53, 49 and 54; SEQ ID NOs.: 55, 56, 57, 58, 59 and 44; SEQ ID NOs.: 61, 46, 63, 58, 49 and 44; SEQ ID NOs.: 64, 65, 66, 67, 68 and 69; SEQ ID NOs.: 70, 71, 72, 73, 74 and 75; SEQ ID NOs.: 76, 77, 78, 79, 80 and 75; SEQ ID NOs.: 81, 82, 83, 84, 85 and 75; SEQ ID NOs.: 85, 86, 87, 88, 89 and 90; SEQ ID NOs.: 85, 91, 92, 93, 94 and 90; SEQ ID NOs.: 85, 96, 92, 93, 94 and 90; SEQ ID NOs.: 85, 86, 87, 97, 98 and 90; SEQ ID NOs.: 85, 99, 100, 101, 102 and 95; SEQ ID NOs.: 85, 99, 100, 101, 102 and 103; SEQ ID NOs.: 85, 99, 100, 104, 102 and 90; SEQ ID NOs.: 85, 105, 92, 93, 94 and 90; SEQ ID NOs.: 85, 99, 100, 101, 102 and 107; SEQ ID NOs.: 108, 109, 110, 111, 112, 113; SEQ ID NOs.: 108, 114, 115, 111, 116 and 117; or SEQ ID NOs.: 108, 118, 119, 111, 120 and 121 (CDRs according to Kabat).

In some embodiments, the anti-HIV gp120 V3 glycan-directed antibody or antigen-binding fragment thereof comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 123, 124, 9, 10, 11 and 12; SEQ ID NOs.: 125, 126, 16, 17, 11 and 18; SEQ ID NOs.: 127, 128, 20, 17, 11 and 18; SEQ ID NOs.: 129, 130, 23, 24, 25 and 26; SEQ ID NOs.: 129, 130, 27, 24, 25 and 26; SEQ ID NOs.: 131, 132, 30, 31, 32 and 33; SEQ ID NOs.: 133, 134, 36, 37, 25 and 38; SEQ ID NOs.: 135, 136, 41, 42, 43 and 44; SEQ ID NOs.: 137, 138, 47, 48, 49 and 50; SEQ ID NOs.: 137, 138, 52, 53, 49 and 54; SEQ ID NOs.: 139, 56, 57, 58, 59 and 44; SEQ ID NOs.: 141, 138, 63, 58, 49 and 44; SEQ ID NOs.: 142, 143, 66, 67, 68 and 69; SEQ ID NOs.: 144, 145, 72, 73, 74 and 75; SEQ ID NOs.: 146, 147, 78, 79, 80 and 75; SEQ ID NOs.: 146, 147, 83, 84, 85 and 75; SEQ ID NOs.: 149, 150, 87, 88, 89 and 90; SEQ ID NOs.: 151, 150, 87, 88, 89 and 90; SEQ ID NOs.: 152, 153, 92, 93, 94 and 90; SEQ ID NOs.: 151, 150, 87, 97, 98 and 90; SEQ ID NOs.: 152, 153, 100, 101, 102 and 95; SEQ ID NOs.: 152, 153, 100, 101, 102 and 103; SEQ ID NOs.: 152, 153, 100, 104, 102 and 90; SEQ ID NOs.: 152, 153, 100, 101, 102 and 107; SEQ ID NOs.: 154, 155, 110, 111, 116 and 117; SEQ ID NOs.: 156, 157, 115, 111, 116 and 117; or SEQ ID NOs.: 158, 159, 119, 111, 120 and 121 (CDRs according to Chothia).

In some embodiments, the anti-HIV gp120 V3 glycan-directed antibody or antigen-binding fragment thereof comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 160, 161, 162, 163, 164 and 12; SEQ ID NOs.: 165, 166, 167, 168, 164 and 18; SEQ ID NOs.: 165, 166, 461, 168, 164 and 18; SEQ ID NOs.: 169, 170, 171, 168, 164 and 18; SEQ ID NOs.: 172, 173, 174, 175, 164 and 26; SEQ ID NOs.: 172, 173, 176, 175, 164 and 26; SEQ ID NOs.: 177, 178, 179, 180, 164 and 38; SEQ ID NOs.: 181, 182, 183, 184, 185 and 33; SEQ ID NOs.: 186, 187, 188, 189, 190 and 44; SEQ ID NOs.: 191, 192, 193, 194, 195 and 50; SEQ ID NOs.: 191, 196, 197, 198, 195 and 54; SEQ ID NOs.: 199, 200, 201, 202, 399 and 44; SEQ ID NOs.: 203, 204, 205, 202, 195 and 44; SEQ ID NOs.: 206, 207, 208, 209, 210 and 69; 211, 212, 213, 214, 215 and 75; SEQ ID NOs.: 216, 217, 218, 219, 220 and 75; SEQ ID NOs.: 221, 217, 83, 223, 224 and 75; SEQ ID NOs.: 225, 226, 87, 227, 228 and 90; SEQ ID NOs.: 229, 226, 87, 227, 228 and 90; SEQ ID NOs.: 230, 231, 92, 232, 233 and 90; SEQ ID NOs.: 230, 234, 92, 232, 233 and 90; SEQ ID NOs.: 229, 226, 87, 235, 398 and 90; SEQ ID NOs.: 230, 236, 100, 232, 233 and 95; SEQ ID NOs.: 230, 236, 100, 232, 233 and 103; SEQ ID NOs.: 230, 236, 100, 237, 233 and 90; SEQ ID NOs.: 230, 238, 92, 232, 233 and 107; SEQ ID NOs.: 239, 240, 110, 241, 242 and 113; SEQ ID NOs.: 243, 244, 115, 241, 245 and 117; or SEQ ID NOs.: 243, 246, 119, 241, 247 and 121 (CDRs according to IMGT).

In some embodiments, the anti-HIV gp120 V3 glycan-directed antibody or antigen-binding fragment thereof comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 248, 249, 250, 251, 252 and 253; SEQ ID NOs.: 248, 254, 250, 251 252 and 253; SEQ ID NOs.: 255, 256, 257, 258, 252 and 259; SEQ ID NOs.: 260, 261, 262, 258, 252 and 259; SEQ ID NOs.: 263, 264, 265, 266, 267 and 268; SEQ ID NOs.: 263, 264, 397, 266, 267 and 268; SEQ ID NOs.: 269, 270, 271, 272, 273 and 274; SEQ ID NOs.: 275, 276, 277, 278, 279 and 280; SEQ ID NOs.: 281, 282, 283, 284, 285 and 286; SEQ ID NOs.: 287, 288, 289, 290, 291 and 286; SEQ ID NOs.: 287, 292, 293, 294, 295 and 296; SEQ ID NOs.: 297, 298, 299, 300, 301 and 286; SEQ ID NOs.: 302, 288, 303, 300, 295 and 286; SEQ ID NOs.: 304, 305, 306, 307, 308 and 309; SEQ ID NOs.: 310, 311, 312, 313, 314 and 315; SEQ ID NOs.: 316, 316, 318, 319, 320 and 315; SEQ ID NOs.: 321, 322, 323, 324, 325 and 315; 326, 327, 328, 329, 330 and 331; SEQ ID NOs.: 332, 327, 328, 329, 330 and 331; SEQ ID NOs.: 333, 334, 335, 336, 337 and 338; SEQ ID NOs.: 333, 339, 335, 336, 337 and 338; SEQ ID NOs.: 332, 327, 328, 340, 341 and 338; SEQ ID NOs.: 342, 343, 344, 336, 345 and 346; SEQ ID NOs.: 342, 343, 344, 336, 347 and 348; SEQ ID NOs.: 342, 343, 344, 349, 350 and 338; SEQ ID NOs.: 333, 351, 335, 336, 337 and 338; SEQ ID NOs.: 342, 343, 344, 336, 347 and 352; SEQ ID NOs: 353, 354, 355, 356, 357 and 358; SEQ ID NOs: 359, 360, 361, 356, 362 and 363; or SEQ ID NOs: 359, 364, 365, 356, 366 and 358. (CDRs according to Honegger).

Illustrative embodiments of CDR sequences of an anti-HIV gp120 V3 glycan-directed antibody or antigen-binding fragment thereof, useful in the methods described herein, are provided in Tables A1-A4.

In some embodiments, the anti-HIV gp120 V3 glycan-directed antibody or antigen-binding fragment thereof comprises VH and VL comprising amino acid sequences that are at least 80%, at least 85%, at least 90%, at least 91%, at least 92%, at least 93%, at least 94%, at least 95%, at least 96%, at least 97%, at least 98%, at least 99%, or 100%, identical to the amino acid sequences set forth, respectively, as selected from: SEQ ID NOs.: 400 and 401; SEQ ID NOs.: 402 and 403; SEQ ID NOs.: 402 and 404; SEQ ID NOs.: 462 and 463; SEQ ID NOs.: 464 and 465; SEQ ID NOs.: 405 and 406; SEQ ID NOs.: 466 and 467; SEQ ID NOs.: 407 and 408; SEQ ID NOs.: 409 and 410; SEQ ID NOs.: 411 and 412; SEQ ID NOs.: 413 and 414; SEQ ID NOs.: 415 and 416; SEQ ID NOs.: 417 and 418; SEQ ID NOs.: 419 and 420; SEQ ID NOs.: 421 and 422; SEQ ID NOs.: 423 and 424; SEQ ID NOs.: 425 and 426; SEQ ID NOs.: 427 and 428; SEQ ID NOs.: 429 and 430; SEQ ID NOs.: 431 and 432; SEQ ID NOs.: 433 and 434; SEQ ID NOs.: 435 and 436; SEQ ID NOs.: 437 and 438; SEQ ID NOs.: 439 and 440; SEQ ID NOs.: 441 and 442; SEQ ID NOs.: 443 and 444; SEQ ID NOs.: 445 and 446; SEQ ID NOs.: 447 and 448; SEQ ID NOs.: 449 and 450; SEQ ID NOs.: 451 and 452; SEQ ID NOs.: 453 and 454; SEQ ID NOs.: 455 and 456; SEQ ID NOs.: 457 and 458; or SEQ ID NOs.: 459 and 460. Illustrative embodiments of variable domain VH and VL sequences of an anti-HIV gp120 V3 glycan-directed antibody or antigen-binding fragment thereof, useful in the methods described herein, are provided in Table B.

In some embodiments, the anti-HIV gp120 V3 glycan directed antibody comprises VH and VL amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth below, respectively, and comprising one or more of the following amino acids at the indicated positions (position numbering according to Kabat): SEQ ID NOs.: 400 or 402, comprising one or more of: Ser-Ser-Val (SSV) or Thr-Gly-Val (TGV) at positions 82a-82c, Gln (Q) at position 39, Asn (N) at position 60, His (H) at position 68, any one of Lys (K), His (H) or Thr (T) at position 105, Leu (L) at position 2, Ala (A) at position 32, and/or Ala (A) at position 95; and SEQ ID NOs.: 401, 403 or 404 comprising one or more of: Gly (G) at position 67, Tyr (Y), Phe (F) or Thr (T) at position 67a, Arg (R) at position 67b, Pro (P) at position 67c, and/or Lys (K) at position 103. In some embodiments, the anti-HIV gp120 V3 glycan directed antibody comprises VH and VL amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth below, respectively, and comprising one or more of the following amino acids at the indicated positions (position numbering according to Kabat): SEQ ID NO.: 400 or 402, comprising one or more of: Thr-Gly-Val (TGV) at positions 82a-82c, Asn (N) at position 60, His (H) at position 68, any one of Lys (K), His (H) and/or Thr (T) at position 105; and SEQ ID NOs.: 401, 403 or 404 comprising one or more of: Gly (G) at position 67, Tyr (Y), Phe (F) or Thr (T) at position 67a, Arg (R) at position 67b, Pro (P) at position 67c.

Fc Mutations that Increase Serum Half-Life

In some embodiments, the Fc region or Fc domain of the anti-HIV gp120 V3 glycan directed antibody comprise amino acid modifications that promote an increased serum half-life of the anti-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). Sec, e.g., U.S. Pat. No. 7,658,921. This type of mutant, designated as a “YTE mutant” exhibits a four-fold increased half-life relative to wild-type versions of the same antibody (Dall'Acqua, et al., J Biol Chem, 281:23514-24 (2006); Robbie, et al., Antimicrob Agents Chemotherap., 57 (12): 6147-6153 (2013)). In certain embodiments, the Fc region or Fc domain of one or both of the CD3-targeting heavy chain and the HIV antigen-targeting heavy chain comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen binding molecule. In other embodiments, the Fc region or Fc domain of one or both of the CD3-targeting heavy chain and the HIV antigen-targeting heavy chain comprise a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region or Fc domain of one or both of the CD3-targeting heavy chain and the HIV antigen-targeting heavy chain comprise H433K and N434F (EU numbering) mutations.

Fc Mutations that Enhance Effector Activity

In some embodiments, the Fc region or Fc domain of the anti-HIV gp120 V3 glycan directed antibody comprise post-translational and/or amino acid modifications that increase effector activity, e.g., have improved FcγIIIa binding and increased antibody-dependent cellular cytotoxicity (ADCC). In some embodiments, the Fc region or Fc domain of the anti-HIV gp120 V3 glycan directed antibody comprises DE modifications (i.e., S239D and 1332E by EU numbering) in the Fc region. In some embodiments, the Fc region or Fc domain of the anti-HIV gp120 V3 glycan directed antibody comprises DEL modifications (i.e., S239D, 1332E and A330L by EU numbering) in the Fc region. In some embodiments, the Fc region or Fc domain of the anti-HIV gp120 V3 glycan directed antibody comprises DEA modifications (i.e., S239D, 1332E and G236A by EU numbering) in the Fc region. In some embodiments, the Fc region or Fc domain of the anti-HIV gp120 V3 glycan directed antibody comprises DEAL modifications (i.e., S239D, 1332E, G236A and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved FcγIIIa binding and increased antibody-dependent cellular cytotoxicity (ADCC) include without limitation (EU numbering) F243L/R292P/Y300L/V305I/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase Clq binding and complement-dependent cytotoxicity (CDC) include without limitation (EU numbering) S267E/H268F/S324T or K326W/E333S. Fc region mutations that enhance effector activity are reviewed in, e.g., Wang, et al., Protein Cell (2018) 9 (1): 63-73; and Saunders, Front Immunol. (2019) 10:1296.

In other embodiments, the anti-HIV gp120 V3 glycan directed antibody or antigen-binding fragment thereof has modified glycosylation, which, e.g., may be introduced post-translationally or through genetic engineering. In some embodiments, the anti-HIV gp120 V3 glycan directed antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. Afucosylated monoclonal antibodies are monoclonal antibodies engineered so that the oligosaccharides in the Fc region of the antibody do not have any fucose sugar units.

In some embodiments, as appropriate, the Fc region or Fc domain of the anti-HIV gp120 V3 glycan directed antibody can comprise post-translational and/or amino acid modifications for increasing serum half-life and enhancing effector activity.

4. Combination Therapies with Two or More Anti-HIV Antibodies

In certain embodiments, this disclosure provides a method for treating or preventing an HIV infection in a human subject having, or at risk of having, the HIV infection. The method comprises administering to the human subject a therapeutically effective amount of an anti-HIV gp120 V3 glycan directed antibody or antigen-binding fragment, as disclosed herein, or a pharmaceutical composition thereof, in combination with a therapeutically effective amount of one or more (e.g., one, two, three, one or two, or one to three) additional therapeutic agents. In one embodiment, a method for treating an HIV infection in a human subject having or at risk of having the infection is provided, the method comprising administering to the human subject a therapeutically effective amount of an antibody or antibodies disclosed herein, or a pharmaceutically acceptable salt thereof, in combination with a therapeutically effective amount of one or more (e.g., one, two, three, one or two, or one to three) additional therapeutic agents.

### Antibody Combination Therapy

In some embodiments, the anti-V3-glycan antibody or antigen-binding fragment thereof is co-administered with a second anti-HIV antibody. In some embodiments, the anti-V3-glycan antibody or antigen-binding fragment thereof is co-administered with a second anti-HIV antibody that binds to an epitope or region of gp 120 selected from the group consisting of: (i) second variable loop (V2) and/or Env trimer apex; (ii) CD4 binding site (CD4bs); (iii) gp120/gp41 interface; or (v) silent face of gp120. The foregoing epitopes or regions of gp 120 bound by broadly neutralizing antibodies are described, e.g., in McCoy, Retrovirology (2018) 15:70; Sok and Burton, Nat Immunol. 2018 19 (11): 1179-1188; Possas, et al., Expert Opin Ther Pat. 2018 July; 28 (7): 551-560; and Stephenson and Barouch, Curr HIV/AIDS Rep (2016) 13:31-37, which are hereby incorporated herein by reference in their entirety for all purposes.

In some embodiments, the combination therapy entails co-administration of an anti-V3-glycan antibody or antigen-binding fragment thereof and another anti-HIV broadly neutralizing antibody or bNAb (i.e., a neutralizing antibody that neutralizes multiple HIV-1 viral strains). Various bNAbs are known in the art and may be used as a combining therapeutic agent. Additional illustrative bNAbs of use include, those that comprise VH and VL that bind to or compete with an epitope or region of gp 120 selected from the group consisting of: (i) second variable loop (V2) and/or Env trimer apex; (ii) CD4 binding site (CD4bs); (iii) gp120/gp41 interface; or (v) silent face of gp120. Illustrative bNAbs for use in anti-HIV antibody combination therapies include those that comprise VH and VL that bind to or compete with 2F5, 4E10, M66.6, CAP206-CH12, 10E8, 10E8v4, 10E8-5R-100° F., DH511.11P, 7b2, and LN01 (all of which bind the MPER of gp41); PG9, PG16, CH01-04 (all of which bind V1V2-glycan), 2G12 (which binds to outer domain glycan); b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N6LS (VRC-HIVMAB091-00-AB), N49-P7, NC-Cow1, IOMA, CH235 and CH235.12, N49P6, N49P7, N49P11, N49P9 and N60P25 (all of which bind to the CD4 binding site).

In some embodiments, the combination therapy includes an antibody that binds to an epitope or region of gp 120 in the second variable loop (V2) and/or Env trimer apex and competes with or comprises CDRs and/or VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01.

In some embodiments, the combination therapy includes an antibody that binds to an epitope or region of gp 120 in the CD4 binding site (CD4bs) and competes with or comprises CDRs and/or VH and VL regions from an antibody selected from the group consisting of b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N6LS (VRC-HIVMAB091-00-AB), N49-P7, NC-Cow1, IOMA, CH235 and CH235.12, N49P6, N49P7, N49P11, N49P9 and N60P25.

In some embodiments, the combination therapy includes an antibody that binds to an epitope or region of gp 120 in the gp120/gp41 interface and competes with or comprises CDRs and/or VH and VL regions from an antibody selected from the group consisting of PGT-151, CAP248-2B, 35022, 8ANC195, ACS202, VRC34 and VRC34.01.

In some embodiments, the combination therapy includes an antibody that binds to an epitope or region of the gp 120 silent face and competes with or comprises second VH and VL regions from antibody VRC-PG05.

In some embodiments, the combination therapy includes an antibody that binds to an epitope or region of gp41 in the membrane proximal region (MPER) and competes with or comprises second VH and VL regions from an antibody selected from the group consisting of 10E8, 10E8v4, 10E8-5R-100° F., 4E10, DH511.11P, 2F5, 7b2, and LN01. In some embodiments, the combination therapy includes an antibody that binds to an epitope or region of KLIC (“KLIC” disclosed as SEQ ID NO: 468), an immutable site of the transmembrane protein gp41 and competes with or comprises second VH and VL regions from Clone 3 human monoclonal antibody (C13hmAb) (Protheragen). See, e.g., Vanini, et al., AIDS. (1993) 7 (2): 167-74.

In some embodiments, the combination therapy includes an antibody that binds to and epitope or region of the gp41 fusion peptide and competes with or comprises second VH and VL regions from an antibody selected from the group consisting of VRC34 and ACS202.

In some embodiments, the combination therapy includes a multi-specific, e.g., a bispecific or tri-specific antibody that binds to an HIV antigen. Examples of HIV bispecific and trispecific antibodies include MGD014, B12BiTe, BiIA-SG, TMB-bispecific, SAR-441236, VRC-01/PGDM-1400/10E8v4, 10E8.4/iMab, and 10E8v4/PGT121-VRC01.

Prior to administration, the bNAbs may be improved to have enhanced drug-like-properties, reduced immunogenicity, enhanced ADCC, and suitable pharmacokinetic properties. Such antibodies were shown to bind to the HIV envelope glycoprotein expressed on the surface of virion or infected cells, and mediate both direct neutralization of the virus as well as potent NK, Monocyte and PBMC killing of these cells. This property allows the antibodies to treat HIV infections by neutralizing the virus, and also kill and eliminate latently HIV infected cells in infected individuals, potentially leading to a sterilizing cure for HIV.

In various embodiments, all antibodies administered in a combination anti-HIV antibody therapy can have Fc and/or post-translational modifications that increase serum half-life and/or enhance effector activity, as described above.

In various embodiments, the anti-HIV gp120 V3 glycan directed antibody or antigen-binding fragments, and optionally combined bNAbs, can be in vivo delivered, e.g., expressed in vivo from administered mRNA or engineered B-cells. Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

5. Combination Therapies with Other Anti-HIV Therapeutic Agents

In certain embodiments, a method for treating or preventing an HIV infection in a human having or at risk of having the infection is provided, comprising administering to the human a therapeutically effective amount of the anti-HIV gp120 V3 glycan directed antibody or antigen-binding fragments, as disclosed herein, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of the anti-HIV gp120 V3 glycan directed antibody or antigen-binding fragments, as disclosed herein, in combination with a therapeutically effective amount of one or more (e.g., one, two, three, one or two, or one to three) additional therapeutic agents.

In one embodiment, pharmaceutical compositions comprising the anti-HIV gp120 V3 glycan directed antibody or antigen-binding fragments, as disclosed herein, in combination with one or more (e.g., one, two, three, one or two, or one to three) additional therapeutic agents, and a pharmaceutically acceptable carrier, diluent, or excipient are provided.

In certain embodiments, provided are methods for treating an HIV infection, comprising administering to a patient in need thereof a therapeutically effective amount of the anti-HIV gp120 V3 glycan directed antibody or antigen-binding fragment thereof, as described herein, in combination with a therapeutically effective amount of one or more additional therapeutic agents which are suitable for treating an HIV infection.

In certain embodiments, the anti-HIV gp120 V3 glycan directed antibody or antigen-binding fragment thereof is combined with one, two, three, four, or more additional therapeutic agents. In certain embodiments, the anti-HIV gp120 V3 glycan directed antibody or antigen-binding fragment thereof is combined with two additional therapeutic agents. In other embodiments, the anti-HIV gp120 V3 glycan directed antibody or antigen-binding fragment thereof is combined with three additional therapeutic agents. In further embodiments, the anti-HIV gp120 V3 glycan directed antibody or antigen-binding fragment thereof is combined with four additional therapeutic agents. The one, two, three, four, or more additional therapeutic agents can be different therapeutic agents selected from the same class of therapeutic agents, (e.g., one or more anti-HIV broadly neutralizing antibodies), and/or they can be selected from different classes of therapeutic agents.

### Administration of HIV Combination Therapy

In certain embodiments, the anti-HIV gp120 V3 glycan directed antibody or antigen-binding fragment thereof, as described herein, is co-administered with one or more additional therapeutic agents. Co-administration of an anti-HIV gp120 V3 glycan directed antibodies or antigen-binding fragments disclosed herein with one or more additional therapeutic agents generally refers to simultaneous or sequential administration of an anti-HIV gp120 V3 glycan directed antibodies or antigen-binding fragments disclosed herein and one or more additional therapeutic agents, such that therapeutically effective amounts of the anti-HIV gp120 V3 glycan directed antibodies or antigen-binding fragments disclosed herein and the one or more additional therapeutic agents are both present in the body of the patient. When administered sequentially, the combination may be administered in two or more administrations.

Co-administration includes concurrent administration as well as administration of unit dosages of the anti-HIV gp120 V3 glycan directed antibody or antigen-binding fragment thereof, as described herein before or after administration of unit dosages of one or more additional therapeutic agents. For example, the anti-HIV gp120 V3 glycan directed antibody or antigen-binding fragment thereof, as described herein, may be administered within seconds, minutes, hours or days of the administration of the one or more additional therapeutic agents. In some embodiments, a unit dose of an anti-HIV gp120 V3 glycan directed antibodies or antigen-binding fragments disclosed herein is administered first, followed within seconds, minutes, hours or days by administration of a unit dose of one or more additional therapeutic agents. Alternatively, a unit dose of one or more additional therapeutic agents is administered first, followed by administration of a unit dose of an anti-HIV gp120 V3 glycan directed antibodies or antigen-binding fragments disclosed herein within seconds, minutes, hours or days. In other embodiments, a unit dose of an anti-HIV gp120 V3 glycan directed antibodies or antigen-binding fragments disclosed herein is administered first, followed, after a period of hours (e.g., 1-12 hours, 1-24 hours, 1-36 hours, 1-48 hours, 1-60 hours, 1-72 hours), by administration of a unit dose of one or more additional therapeutic agents. In yet other embodiments, a unit dose of one or more additional therapeutic agents is administered first, followed, after a period of hours (e.g., 1-12 hours, 1-24 hours, 1-36 hours, 1-48 hours, 1-60 hours, 1-72 hours), by administration of a unit dose of an anti-HIV gp120 V3 glycan directed antibodies or antigen-binding fragments disclosed herein.

In certain embodiments, an anti-HIV gp120 V3 glycan directed antibodies or antigen-binding fragments disclosed herein is combined with one or more additional therapeutic agents in a unitary dosage form for simultaneous administration to a patient, for example as a solid, liquid or suspension dosage form for oral, intravenous, intramuscular or subcutaneous administration.

In certain embodiments, the anti-HIV gp120 V3 glycan directed antibodies or antigen-binding fragments are formulated as a liquid solution or suspension which may optionally contain one or more other compounds useful for treating HIV. In certain embodiments, the liquid solution or suspension can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, such liquid solutions or suspensions are suitable for once daily, once weekly (i.e., QW), once bi-weekly (i.e. once every other week, or once every two weeks or Q2W), once monthly (i.e., QM) or once bi-monthly dosing (i.e. once every other month, or once every two months or Q2M) dosing or administration intervals. In some embodiments, the anti-HIV gp120 V3 glycan directed antibodies or antigen-binding fragments are administered once daily, once weekly (i.e., QW), once bi-weekly (i.e. once every other week, or once every two weeks or Q2W), once monthly (i.e., QM), once bi-monthly dosing (i.e. once every other month, or once every two months or Q2M), once every three months (i.e., Q3M), once every four months (i.e., Q4M),

### HIV Combination Therapy

In the above embodiments, the additional therapeutic agent may be an anti-HIV agent. HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, (e.g., immunostimulators), immunotherapeutic agents, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies), latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, TAT protein inhibitors, HIV-1 Nef modulators (e.g., Nef inhibitors), Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-4 inhibitors, CDK-6 inhibitors, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, TAT protein inhibitors, prolylendopeptidase inhibitors, Phospholipase A2 inhibitors, pharmacokinetic enhancers, HIV gene therapy, TNF alpha ligand inhibitors, IFN antagonists, HIV vaccines, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, HIV capsid inhibitors, HIV Tat or Rev inhibitors, immunomodulators, (e.g., immunostimulators), immunotherapeutic agents, immune-based therapies, PI3K inhibitors, HIV antibodies, and bispecific antibodies, and “antibody-like” therapeutic proteins, and combinations thereof.

### HIV Combination Drugs

In certain embodiments, the anti-HIV gp120 V3 glycan directed antibodies or antigen-binding fragments described herein are combined with one, two, three, four or more additional anti-HIV therapeutic agents. Example anti-HIV therapeutic agents that can be combined include without limitation ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir alafenamide and elvitegravir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; cabotegravir+rilpivirine; elpida (elsulfavirine; VM-1500; VM-1500A); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; efavirenz, lamivudine, and emtricitabine; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

### Other HIV Drugs

Examples of other drugs for treating HIV that can be combined with an agent of this disclosure include aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-cCD4-Ig gene therapy, MazF gene therapy, BlockAide, bevirimat derivatives, ABX-464, AG-1105, APH-0812, bryostatin analogs, BIT-225, CYT-107, CS-TATI-1, fluoro-beta-D-arabinose nucleic acid (FANA)-modified antisense oligonucleotides, FX-101, griffithsin, HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, M1-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, rfhSP-D, [¹⁸F]-MC-225, URMC-099-C, RES-529, and VIR-576.

### HIV Protease Inhibitors

In certain embodiments, the anti-HIV gp120 V3 glycan directed antibodies or antigen-binding fragments described herein are combined with an HIV protease inhibitor. Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, AEBL-2, DG-17, GS-1156, TMB-657 (PPL-100), T-169, BL-008, MK-8122, TMB-607, GRL-02031 and TMC-310911.

### HIV Ribonuclease H Inhibitors

In certain embodiments, the anti-HIV gp120 V3 glycan directed antibodies or antigen-binding fragments described herein are combined with an HIV ribonuclease H inhibitor. Examples of HIV ribonuclease H inhibitors that can be combined include NSC-727447.

### HIV Nef Inhibitors

In certain embodiments, the anti-HIV gp120 V3 glycan directed antibodies or antigen-binding fragments described herein are combined with an HIV Nef inhibitor. Examples of HIV Nef inhibitors that can be combined with include FP-1.

### HIV Reverse Transcriptase Inhibitors

In certain embodiments, the anti-HIV gp120 V3 glycan directed antibodies or antigen-binding fragments described herein are combined with a non-nucleoside or non-nucleotide inhibitor. Examples of HIV non-nucleoside or non-nucleotide inhibitors of reverse transcriptase include dapivirine, delavirdine, delavirdine mesylate, doravirine, efavirenz, etravirine, lentinan, nevirapine, rilpivirine, ACC-007, ACC-008, AIC-292, F-18, KM-023, PC-1005, VM-1500A-LAI, PF-3450074, elsulfavirine (sustained release oral, HIV infection), elsulfavirine (long-acting injectable nanosuspension, HIV infection), and elsulfavirine (VM-1500).

In certain embodiments, the anti-HIV gp120 V3 glycan directed antibodies or antigen-binding fragments described herein are combined with an HIV nucleoside or nucleotide inhibitor. Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500 and KP-1461.

### HIV Integrase Inhibitors

In certain embodiments, the anti-HIV gp120 V3 glycan directed antibodies or antigen-binding fragments described herein are combined with an HIV integrase inhibitor. Examples of HIV integrase inhibitors include elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T-169, STP-0404, VM-3500 and cabotegravir.

In certain embodiments, the anti-HIV gp120 V3 glycan directed antibodies or antigen-binding fragments described herein are combined with a HIV non-catalytic site, or allosteric, integrase inhibitor (NCINI). Examples of HIV non-catalytic site, or allosteric, integrase inhibitors (NCINI) include CX-05045, CX-05168, and CX-14442.

### HIV Entry Inhibitors

In certain embodiments, the anti-HIV gp120 V3 glycan directed antibodies or antigen-binding fragments described herein are combined with an HIV entry inhibitor. Examples of HIV entry (fusion) inhibitors include AAR-501, LBT-5001, cenicriviroc, CCR5 inhibitors, gp41 inhibitors, CD4 attachment inhibitors, gp120 inhibitors, gp160 inhibitors and CXCR4 inhibitors.

In certain embodiments, the anti-HIV gp120 V3 glycan directed antibodies or antigen-binding fragments described herein are combined with a CCR5 inhibitor. Examples of CCR5 inhibitors include aplaviroc, vicriviroc, maraviroc, maraviroc (long-acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

In certain embodiments, the anti-HIV gp120 V3 glycan directed antibodies or antigen-binding fragments described herein are combined with a CXCR4 inhibitor. Examples of CXCR4 inhibitors include plerixafor, ALT-1188, N15 peptide, and vMIP (Haimipu).

In certain embodiments, the anti-HIV gp120 V3 glycan directed antibodies or antigen-binding fragments described herein are combined with a gp41 inhibitor. Examples of gp41 inhibitors include albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, PIE-12 trimer and sifuvirtide.

In certain embodiments, the anti-HIV gp120 V3 glycan directed antibodies or antigen-binding fragments described herein are combined with a CD4 attachment inhibitor. Examples of CD4 attachment inhibitors include ibalizumab and CADA analogs

In certain embodiments, the anti-HIV gp120 V3 glycan directed antibodies or antigen-binding fragments described herein are combined with a gp 120 inhibitor. Examples of gp 120 inhibitors include anti-HIV microbicide, Radha-108 (receptol) 3B3-PE38, BanLec, bentonite-based nanomedicine, fostemsavir tromethamine, IQP-0831, VVX-004, and BMS-663068.

In certain embodiments, the anti-HIV gp120 V3 glycan directed antibodies or antigen-binding fragments described herein are combined with a gp160 inhibitor. Examples of gp 160 inhibitors that can be combined include fangchinoline.

### HIV Maturation Inhibitors

In certain embodiments, the anti-HIV gp120 V3 glycan directed antibodies or antigen-binding fragments described herein are combined with an HIV maturation inhibitor. Examples of HIV maturation inhibitors include BMS-955176, GSK-3640254 and GSK-2838232.

### Latency Reversing Agents

In certain embodiments, the anti-HIV gp120 V3 glycan directed antibodies or antigen-binding fragments described herein are combined with an HIV latency reversing agent. Examples of latency reversing agents that can be combined with the one or more multi-specific antigen binding molecules, described herein, include IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); toll-like receptor (TLR) agonists (including TLR7 agonists, e.g., GS-9620 and TLR8 agonists, e.g., GS-9688), histone deacetylase (HDAC) inhibitors, proteasome inhibitors such as velcade, protein kinase C (PKC) activators, Smyd2 inhibitors, BET-bromodomain 4 (BRD4) inhibitors, ionomycin, IAP antagonists (inhibitor of apoptosis proteins, such as APG-1387, LBW-242), SMAC mimetics (including TL32711, LCL161, GDC-0917, HGS1029, AT-406), Debio-1143, PMA, SAHA (suberanilohydroxamic acid, or suberoyl, anilide, and hydroxamic acid), NIZ-985, IL-15 modulating antibodies, (including IL-15, IL-15 fusion proteins and IL-15 receptor agonists, e.g., ALT-803), JQ1, disulfiram, amphotericin B, and ubiquitin inhibitors such as largazole analogs, APH-0812, and GSK-343. Examples of HDAC inhibitors include romidepsin, vorinostat, and panobinostat. Examples of PKC activators include indolactam, prostratin, ingenol B, and DAG-lactones.

### Toll-Like Receptor (TLR) Agonists

In certain embodiments, the anti-HIV gp120 V3 glycan directed antibodies or antigen-binding fragments described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR 10 (NCBI Gene ID: 81793). Example TLR7 agonists that can be co-administered or combined with the one or more multi-specific antigen binding molecules, described herein, include without limitation AL-034, DSP-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262, telratolimod and BDB-001. Example TLR8 agonists that can be co-administered or combined with the one or more multi-specific antigen binding molecules, described herein, include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091.

### Histone Deacetylase (HDAC) Inhibitors

In certain embodiments, the anti-HIV gp120 V3 glycan directed antibodies or antigen-binding fragments described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Cyclin-Dependent Kinase (CDK) Inhibitors or Antagonists

In certain embodiments, the anti-HIV gp120 V3 glycan directed antibodies or antigen-binding fragments described herein are combined with an inhibitor or antagonist of a cyclin-dependent kinase (CDK), e.g., cyclin dependent kinase 4 (CDK4; NCBI Gene ID: 1019), cyclin dependent kinase 6 (CDK6; NCBI Gene ID: 1021), cyclin dependent kinase 9 (CDK9; NCBI Gene ID: 1025). In some embodiments, the CDK4/CDK6/CDK9 inhibitor or antagonist is selected from the group consisting of VS2-370.

### Stimulator of Interferon Genes (STING) Agonists

In some embodiments, the anti-HIV gp120 V3 glycan directed antibodies or antigen-binding fragments described herein are combined with an stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

### RIG-I Agonists

In certain embodiments, the anti-HIV gp120 V3 glycan directed antibodies or antigen-binding fragments described herein are combined with an agonist of DExD/H-box helicase 58 (DDX58; a.k.a., RIG-I, RIG1, RIGI, RLR-1, SGMRT2; NCBI Gene ID: 23586). In some embodiments, the agents described herein are combined with a RIG-I modulator such as RGT-100, or NOD2 modulator, such as SB-9200 (a.k.a., GS 9992; inarigivir), and IR-103. An illustrative RIG-I agonist is KIN1148, described by Hemann, et al., J Immunol May 1, 2016, 196 (1 Supplement) 76.1. Additional RIG-I agonists are described, e.g., in Elion, et al., Cancer Res. (2018) 78 (21): 6183-6195; and Liu, et al., J Virol. (2016) 90 (20): 9406-19. RIG-I agonists are commercially available, e.g., from Invivogen (invivogen.com).

### LAG-3 and TIM-3 Inhibitors

In certain embodiments, the anti-HIV gp120 V3 glycan directed antibodies or antigen-binding fragments described herein are combined with an anti-TIM-3 (a.k.a., hepatitis A virus cellular receptor 2 antibody (HAVCR2; NCBI Gene ID: 84868), such as TSR-022, LY-3321367, MBG-453, INCAGN-2390. In some embodiments, the anti-HIV gp120 V3 glycan directed antibodies or antigen-binding fragments described herein are combined with an anti-LAG-3 (Lymphocyte-activation) (NCBI Gene ID: 3902) antibody, such as relatlimab (ONO-4482), LAG-525, MK-4280, REGN-3767, INCAGN2385.

### Capsid Inhibitors

In certain embodiments, the anti-HIV gp120 V3 glycan directed antibodies or antigen-binding fragments described herein are combined with a capsid inhibitor. Examples of capsid inhibitors that can be combined with an agent of this disclosure include capsid polymerization inhibitors or capsid disrupting compounds, HIV nucleocapsid p7 (NCp7) inhibitors such as azodicarbonamide, HIV p24 capsid protein inhibitors, GS-6207, GS-CA1, AVI-621, AVI-101, AVI-201, AVI-301, and AVI-CAN1-15 series, PF-3450074, and compounds described in Intl. Patent Publ. No. WO 2019/087016.

### Immune-Based Therapies

In certain embodiments, the anti-HIV gp120 V3 glycan directed antibodies or antigen-binding fragments described herein are combined with an immune-based therapy. Examples of immune-based therapies include toll-like receptor (TLR) modulators such as TLR1, TLR2, TLR3, TLR4, TLR5, TLR6, TLR7, TLR8, TLR9, TLR10, TLR11, TLR12, AND TLR13; programmed cell death protein 1 (PD-1) modulators; programmed death-ligand 1 (PD-L1) modulators; IL-15 modulators (e.g., IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethylencimine (PEI); gepon; IL-12; WF-10; VGV-1; MOR-22; BMS-936559; CYT-107, normferon, peginterferon alfa-2a, peginterferon alfa-2b, RPI-MN, STING modulators, RIG-I modulators, NOD2 modulators, SB-9200, and IR-103.

In certain embodiments, the anti-HIV gp 120 V3 glycan directed antibodies or antigen-binding fragments described herein are combined with a TLR agonist. Examples of TLR agonists include without limitation: vesatolimod (GS-9620), lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.

### Immune Checkpoint Receptor Protein Modulators

In various embodiments, the anti-HIV gp120 V3 glycan directed antibodies or antigen-binding fragments described herein are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18 (11): 671-688).

Examples of immune checkpoint proteins or receptors that can be combined with the anti-HIV gp120 V3 glycan directed antibodies or antigen-binding fragments described herein include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript IL (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor DI (KLRD1); and SLAM family member 7 (SLAMF7).

In various embodiments, the anti-HIV gp120 V3 glycan directed antibodies or antigen-binding fragments described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the anti-HIV gp120 V3 glycan directed antibodies or antigen-binding fragments described herein are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). Sec, e.g., Xu, et al., J Exp Clin Cancer Res. (2018) 37:110.

In various embodiments, the anti-HIV gp120 V3 glycan directed antibodies or antigen-binding fragments described herein are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor DI (KLRD1, CD94). In various embodiments, the anti-HIV gp120 V3 glycan directed antibodies or antigen-binding fragments described herein are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). Sec, e.g., Davis, et al., Semin Immunol. (2017) 31:64-75; Fang, et al., Semin Immunol. (2017) 31:37-54; and Chiossone, et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

In various embodiments, the antibodies or antigen-binding fragments as described herein are combined with anti-TIGIT antibodies, such as BMS-986207, RG-6058, AGEN-1307

### TNF Receptor Superfamily (TNFRSF) Member Agonists or Activators

In various embodiments, the anti-HIV gp120 V3 glycan directed antibodies or antigen-binding fragments described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Interleukin Receptor Agonists

In certain embodiments, the anti-HIV gp120 V3 glycan directed antibodies or antigen-binding fragments described herein are combined with an interleukin receptor agonist, such as IL-2, IL-7, IL-15, IL-10, IL-12 agonists; examples of IL-2 receptor agonists such as proleukin (aldesleukin, IL-2); pegylated IL-2 (e.g., NKTR-214); modified variants of IL-2 (e.g., THOR-707), bempegaldesleukin, AIC-284, ALKS-4230, CUI-101, Neo-2/15; IL-15 receptor agonists, such as ALT-803, NKTR-255, and hetIL-15, interleukin-15/Fc fusion protein, AM-0015, NIZ-985, SO-C101, IL-15 Synthorin (pegylated IL-15), P-22339, and a IL-15-PD-1 fusion protein N-809; examples of IL-7 include CYT-107.

Examples of additional interleukin receptor agonists that can be combined with the anti-HIV gp120 V3 glycan directed antibodies or antigen-binding fragments described herein include interferon alfa; interferon alfa-2b; interferon alfa-n3; pegylated interferon alfa; interferon gamma; Flt3 agonists such as CDX-301; gepon; normferon, peginterferon alfa-2a, peginterferon alfa-2b, RPI-MN.

### Bi- and Tri-Specific Natural Killer (NK)-Cell Engagers

In various embodiments, the anti-HIV gp120 V3 glycan directed antibodies or antigen-binding fragments described herein are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (4-1BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRIKE) include OXS-3550, and CD16-IL-15-B7H3 TriKe.

### Phosphatidylinositol 3-Kinase (PI3K) Inhibitors

In certain embodiments, the anti-HIV gp120 V3 glycan directed antibodies or antigen-binding fragments described herein are combined with a PI3K inhibitor. Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, tasclisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

In certain embodiments, the anti-HIV gp120 V3 glycan directed antibodies or antigen-binding fragments described herein are combined with an alpha-4/beta-7 antagonist. Examples of Integrin alpha-4/beta-7 antagonists include PTG-100, TRK-170, abrilumab, etrolizumab, carotegrast methyl, and vedolizumab.

### Pharmacokinetic Enhancers

In certain embodiments, the anti-HIV gp120 V3 glycan directed antibodies or antigen-binding fragments described herein are combined with a pharmacokinetic enhancer. Examples of pharmacokinetic enhancers include cobicistat and ritonavir.

### Additional Therapeutic Agents

Examples of additional therapeutic agents include the compounds disclosed in WO 2004/096286 (Gilead Sciences); WO 2006/015261 (Gilead Sciences); WO 2006/110157 (Gilead Sciences); WO 2012/003497 (Gilead Sciences); WO 2012/003498 (Gilead Sciences); WO 2012/145728 (Gilead Sciences); WO 2013/006738 (Gilead Sciences); WO 2013/159064 (Gilead Sciences); WO 2014/100323 (Gilead Sciences), US 2013/0165489 (University of Pennsylvania), US 2014/0221378 (Japan Tobacco), US 2014/0221380 (Japan Tobacco); WO 2009/062285 (Bochringer Ingelheim); WO 2010/130034 (Bochringer Ingelheim); WO 2013/006792 (Pharma Resources), US20140221356 (Gilead Sciences), US20100143301 (Gilead Sciences) and WO 2013/091096 (Bochringer Ingelheim).

### HIV Combination Therapy

In a particular embodiment, the anti-HIV gp120 V3 glycan directed antibodies or antigen-binding fragments described herein are combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

It will be appreciated by one of skill in the art that the additional therapeutic agents listed above may be included in more than one of the classes listed above. The particular classes are not intended to limit the functionality of those compounds listed in those classes.

In a specific embodiment, the anti-HIV gp120 V3 glycan directed antibodies or antigen-binding fragments described herein are combined with an HIV nucleoside or nucleotide inhibitor of reverse transcriptase and an HIV non-nucleoside inhibitor of reverse transcriptase. In another specific embodiment, the anti-HIV gp120 V3 glycan directed antibodies or antigen-binding fragments described herein are combined with an HIV nucleoside or nucleotide inhibitor of reverse transcriptase, and an HIV protease inhibiting compound. In an additional embodiment, the anti-HIV gp120 V3 glycan directed antibodies or antigen-binding fragments described herein are combined with an HIV nucleoside or nucleotide inhibitor of reverse transcriptase, an HIV non-nucleoside inhibitor of reverse transcriptase, and a pharmacokinetic enhancer. In certain embodiments, the anti-HIV gp120 V3 glycan directed antibodies or antigen-binding fragments described herein are combined with at least one HIV nucleoside inhibitor of reverse transcriptase, an integrase inhibitor, and a pharmacokinetic enhancer. In another embodiment, the anti-HIV gp120 V3 glycan directed antibodies or antigen-binding fragments described herein are combined with two HIV nucleoside or nucleotide inhibitors of reverse transcriptase.

In a particular embodiment, the anti-HIV gp120 V3 glycan directed antibodies or antigen-binding fragments described herein are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In a particular embodiment, the anti-HIV gp120 V3 glycan directed antibodies or antigen-binding fragments described herein are combined with tenofovir, tenofovir disoproxil, tenofovir disoproxil fumarate, tenofovir alafenamide, or tenofovir alafenamide hemifumarate.

In a particular embodiment, the anti-HIV gp120 V3 glycan directed antibodies or antigen-binding fragments described herein are combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In a particular embodiment, the anti-HIV gp120 V3 glycan directed antibodies or antigen-binding fragments described herein are combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In certain embodiments, the anti-HIV gp120 V3 glycan directed antibodies or antigen-binding fragments described herein are combined with one or more additional therapeutic agents in a therapeutically effective dosage amount in the range of e.g., from 1 mg to 50 mg, 75 mg, 100 mg, 150 mg, 200 mg, 250 mg, 300 mg, 400 mg, 500 mg, 1000 mg or 1500 mg of the anti-HIV gp120 V3 glycan directed antibody or antigen-binding fragment. In certain embodiments, the anti-HIV gp120 V3 glycan directed antibodies or antigen-binding fragments described herein are combined with one or more additional therapeutic agents in a therapeutically effective dosage amount in the range of e.g., from about 0.1 mg/kg to about 0.5 mg/kg, 1 mg/kg, 2 mg/kg, 3 mg/kg, 4 mg/kg, 5 mg/kg, 8 mg/kg, 10 mg/kg, 15 mg/kg, 20 mg/kg, 25 mg/kg, 30 mg/kg, 35 mg/kg, 40 mg/kg, 45 mg/kg or 50 mg/kg of the anti-HIV gp120 V3 glycan directed antibody or antigen-binding fragment. In certain embodiments, the anti-HIV gp120 V3 glycan directed antibodies or antigen-binding fragments described herein are combined with one or more additional therapeutic agents in a therapeutically effective dosage amount in the range of e.g., from about 5 mg to about 10 mg, 20 mg, 25 mg, 50 mg, 100 mg, 125 mg, 150 mg, 250 mg, 300 mg, 500 mg, 1000 mg or 1500 mg of the anti-HIV gp120 V3 glycan directed antibody or antigen-binding fragment.

In certain embodiments, the anti-HIV gp120 V3 glycan directed antibodies or antigen-binding fragments described herein are combined with 5-30 mg tenofovir alafenamide fumarate, tenofovir alafenamide hemifumarate, or tenofovir alafenamide, and 200 mg emtricitabine. In certain embodiments, the anti-HIV gp 120 V3 glycan directed antibodies or antigen-binding fragments described herein are combined with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In certain embodiments, the anti-HIV gp120 V3 glycan directed antibodies or antigen-binding fragments described herein are combined with 10 mg tenofovir alafenamide fumarate, tenofovir alafenamide hemifumarate, or tenofovir alafenamide, and 200 mg emtricitabine. In certain embodiments, the anti-HIV gp120 V3 glycan directed antibodies or antigen-binding fragments described herein are combined with 25 mg tenofovir alafenamide fumarate, tenofovir alafenamide hemifumarate, or tenofovir alafenamide, and 200 mg emtricitabine. In some embodiments, the anti-HIV gp120 V3 glycan directed antibodies or antigen-binding fragments described herein are combined with the agents provided herein in any dosage amount of the anti-HIV gp120 V3 glycan directed antibodies or antigen-binding fragments (e.g., from 1 mg to 500 mg of the anti-HIV gp120 V3 glycan directed antibodies or antigen-binding fragments, as described herein) the same as if each combination of dosages were specifically and individually listed.

In certain embodiments, the anti-HIV gp120 V3 glycan directed antibodies or antigen-binding fragments described herein are combined with 200-400 mg tenofovir disoproxil fumarate, tenofovir disoproxil hemifumarate, or tenofovir disoproxil, and 200 mg emtricitabine. In certain embodiments, the anti-HIV gp120 V3 glycan directed antibodies or antigen-binding fragments described herein are combined with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In certain embodiments, the anti-HIV gp120 V3 glycan directed antibodies or antigen-binding fragments described herein are combined with 300 mg tenofovir disoproxil fumarate, tenofovir disoproxil hemifumarate, or tenofovir disoproxil, and 200 mg emtricitabine. The anti-HIV gp120 V3 glycan directed antibodies or antigen-binding fragments may be combined with the agents provided herein in any dosage amount (e.g., from 1 mg to 500 mg of the anti-HIV gp120 V3 glycan directed antibodies or antigen-binding fragments) the same as if each combination of dosages were specifically and individually listed.

### Long-Acting HIV Inhibitors

In some embodiments, the anti-HIV gp120 V3 glycan directed antibodies or antigen-binding fragments described herein can be co-administered with a long-acting HIV inhibitor. Examples of drugs that are being developed as long acting HIV inhibitors include without limitation: cabotegravir LA, rilpivirine LA, any integrase LA, VM-1500 LAI, maraviroc (LAI), tenofovir implant, MK-8591 implant, long-acting dolutegravir.

In one embodiment, kits comprise the anti-HIV gp120 V3 glycan directed antibodies or antigen-binding fragments described herein in combination with one or more (e.g., one, two, three, one or two, or one to three) additional therapeutic agents.

### HIV Vaccines

In certain embodiments, the anti-HIV gp120 V3 glycan directed antibodies or antigen-binding fragments described herein are combined with an HIV vaccine. Examples of HIV vaccines include peptide vaccines, recombinant subunit protein vaccines, live vector vaccines, DNA vaccines, HIV MAG DNA vaccines, CD4-derived peptide vaccines, vaccine combinations, adenoviral vector vaccines (e.g., Ad5, Ad26 or Ad35), simian adenovirus (chimpanzee, gorilla, rhesus i.e., rhAd), adeno-associated virus vector vaccines, chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, bi-segmented or tri-segmented arenavirus based vaccines (e.g., LCMV, Pichinde), trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as Vesicular stomatitis virus (VSV) and marabavirus; recombinant human CMV (rhCMV) based vaccine, alphavirus-based vaccines, such as semliki forest virus, venezuelan equine encephalitis virus and sindbis virus (see, e.g., Lauer, et al., Clin Vaccine Immunol. (2017) 24 (1): e00298-16); LNP formulated mRNA based therapeutic vaccines; and LNP-formulated self-replicating RNA/self-amplifying RNA vaccines.

Examples of HIV vaccines include without limitation anti-CD40.Env-gp140 vaccine, Ad4-EnvC150, BG505 SOSIP.664 gp140 adjuvanted vaccine, BG505 SOSIP.GT1.1 gp 140 adjuvanted vaccine, Chimigen HIV vaccine, ConM SOSIP.v7 gp140, rgp120 (AIDSVAX), ALVAC HIV (vCP1521)/AIDSVAX B/E (gp120) (RV144), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-cnv Clade C+Ad4-mGag), Paxvax, EN41-UGR7C, EN41-FPA2, ENOB-HV-11, Pre VaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp 160 HIV vaccine, RNActive HIV vaccine, SCB-703, Tat Oyi vaccine, TBC-M4, therapeutic HIV vaccine, UBI HIV gp120, Vacc-4×+romidepsin, variant gp 120 polypeptide vaccine, rAd5 gag-pol cnv A/B/C vaccine,. DNA.HTI and MVA.HTI, VRC-HIVDNA016-00-VP+VRC-HIVADV014-00-VP, INO-6145, JNJ-9220, gp145 C.6980; cOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, MVA-BN HIV-1 vaccine regimen, UBI HIV gp120, mRNA based prophylactic vaccines, VPI-211, and TBL-1203HI.

### Birth Control (Contraceptive) Combination Therapy

In certain embodiments, the anti-HIV gp120 V3 glycan directed antibodies or antigen-binding fragments described herein are combined with a birth control or contraceptive regimen. Therapeutic agents used for birth control (contraceptive) include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the anti-HIV gp120 V3 glycan directed antibodies or antigen-binding fragments described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include LB-1903,ENOB-HV-01, GOVX-B01, and SupT1 cell based therapy. Examples of dendritic cell therapy include AGS-004. CCR5 gene editing agents include SB-728T. CCR5 gene inhibitors include Cal-1. In some embodiments, C34-CCR5/C34-CXCR4 expressing CD4-positive T-cells are co-administered with the anti-HIV gp120 V3 glycan directed antibodies or antigen-binding fragments. In some embodiments, the anti-HIV gp120 V3 glycan directed antibodies or antigen-binding fragments are co-administered with AGT-103-transduced autologous T-cell therapy or AAV-eCD4-Ig gene therapy.

### Gene Editors

In certain embodiments, the anti-HIV gp120 V3 glycan directed antibodies or antigen-binding fragments described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T-Cell Therapy

In some embodiments, the anti-HIV gp120 V3 glycan directed antibodies or antigen-binding fragments described herein can be co-administered with a population of immune effector cells engineered to express a chimeric antigen receptor (CAR), wherein the CAR comprises an HIV antigen binding domain. The HIV antigen include an HIV envelope protein or a portion thereof, gp 120 or a portion thereof, a CD4 binding site on gp 120, the CD4-induced binding site on gp120, N glycan on gp 120, the V2 of gp 120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include convertibleCAR-T, VC-CAR-T, anti-CD4 CART-cell therapy, autologous hematopoietic stem cells genetically engineered to express a CD4 CAR and the C46 peptide.

### TCR-T-Cell Therapy

In certain embodiments, the anti-HIV gp120 V3 glycan directed antibodies or antigen-binding fragments described herein are combined with a population of TCR-T-cells. TCR-T-cells are engineered to target HIV derived peptides present on the surface of virus-infected cells.

### 6. Kits

Further provided are kits for performing the diagnostic and treatment methods, as described herein. In some embodiments, the kit comprises primers for amplifying and sequencing at least the gp 120 V3 glycan region of HIV species in a biological sample. In some embodiments, the kit comprises a suite or set of nested primers for amplifying and sequencing at least the gp 120 V3 glycan region of HIV species in a biological sample. In some embodiments, the kit comprises a pair of primers or a set of nested primers for amplifying and sequencing the full length gp120. In some embodiments, the kit comprises sample preparation, nucleic acid quantification, amplification and/or sequencing reagents, e.g., nucleic acid isolation reagents to isolate RNA and/or DNA, protein denaturation solvents, buffers, dNTPs, reverse transcriptase enzyme, polymerase enzyme, and/or detection labels. In some embodiments, the kit comprises library preparation reagents, e.g., barcode reagents and/or target specific primers. In some embodiments, the kit comprises an analysis guide and/or software, e.g., to facilitate practicing the diagnostic methods, described herein. In some embodiments, the kit comprises instructions for sequencing at least the gp 120 V3 glycan region of HIV species in a biological sample and detecting or identifying HIV species expressing a gp 120 comprising: a glycosylated asparagine at the position corresponding to amino acid residue position 332 (N332glycan), an aspartate at the position corresponding to amino acid residue position 325 (D325), and one or more amino acid of: a threonine at the position corresponding to amino acid residue position 63 (T63), a leucine at the position corresponding to amino acid residue position 179 (L179), a threonine at the position corresponding to amino acid residue position 320 (T320), and a histidine at the position corresponding to amino acid residue position 330 (H330), wherein the amino acid positions are with reference to SEQ ID NO: 4 (i.e., residues 1-511 of NCBI Ref Seq No. NP_057856.1), as described herein.

In one embodiment, the kit comprises one or more pharmaceutical packs comprising one or more containers (e.g., vials, ampules, pre-loaded syringes) containing one or more of the ingredients of the pharmaceutical compositions described herein, such as an antibody, or antigen-binding fragment thereof, against the HIV gp120 V3 glycan region, or one or more polynucleotides encoding such antibody or antigen-binding fragment, as provided herein. In some instances, the kits contain a pharmaceutical composition described herein. In some embodiments, the kit comprises one or more containers comprising an antibody, or antigen-binding fragment thereof, against the HIV gp120 V3 glycan region, or one or more polynucleotides encoding such antibody or antigen-binding fragment, in an aqueous solution or in lyophilized form. Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

The following examples are offered to illustrate, but not to limit the claimed invention.

### Example 1

### Identification of HIV-Infected Patients Responsive to Therapy with an Anti-HIV Gp120 V3-Glycan Directed Antibody or Antigen-Binding Fragment Thereof

This Example demonstrates identification of Env genotypes associated with viral susceptibility to neutralization by PGT121 and its derivative, GS-9722 (elipovimab), for prescreening of HIV-infected subjects for susceptibility to PGT121/GS-9722.

High level of sequence diversity in the HIV envelope gene makes prescreening of subjects in clinical trials for broadly neutralizing antibodies (bNAbs) attractive to increase the likelihood of a high response rate. To identify an Env genotype that is predictive of viral susceptibility to PGT121 and GS-9722, we examined the PGT121 and GS-9722 neutralization data and corresponding Env sequence for 206 clade B Envs.

GS-9722 is a engineered variant of PGT121 that maintains the same neutralization activity as PGT121, as evidenced by a highly statistically significant correlation of PGT121 and GS-9722 neutralization IC50s among 397 HIV strains tested with PGT121 and GS-9722 (r²=0.9698, P<0.0001). We therefore combined the GS-9722 neutralization data obtained on 140 clade B Envs isolated from viremic subjects enrolled in Gilead-sponsored clinical trials, with publicly-available PGT121 neutralization data obtained from the Los Alamos HIV Sequence Database (n=66) to increase the statistical power.

Full length Env amino acid sequences were aligned using ClustalW and manually adjusted upon visual inspection. To identify genotypes associated with sensitivity to neutralization by PGT121/GS-9722, we compared the frequency of amino acids and potential N-linked glycosylation sites (PNGS) at each residue among PGT121/GS-9722-sensitive viruses to the frequency in PGT121/GS-9722-resistant viruses by Fisher's exact test. An N-linked glycosylation motif is N-X-S/T, where X is any residue except proline. Neutralization sensitivity to PGT121/GS-9722 was defined as IC50<1 μg/mL. For residues that were statistically significantly associated with sensitivity to PGT121/GS-9722, the positive predictive value (PPV; i.e., probability Env is sensitive to PGT121/GS-9722 when genotype is present) and sensitivity (i.e., probability that the genotype is present when Env is sensitive to PGT121/GS-9722) were calculated as described below:

TABLE 1
2 × 2 Table Used to Calculate PPV, NPV, Sensitivity and Specificity
for Genotypic Determinants of PGT121/GS-9722 Sensitivity

| | PGT121/GS-9722 sensitive | PGT121/GS-9722 resistant |
|---|---|---|
| Genotype (+) | a | c |
| Genotype (−) | b | d |

$PPV = \frac{a}{a + c}$ $Sensitivity = \frac{a}{a + b}$

A Mann-Whitney test was also applied to identify determinants of susceptibility independent of the 1 μg/mL cut-off for defining Envs as “susceptible” vs “resistant”.

Residues that were statistically associated with susceptibility to PGT121/GS-9722 and/or previously are reported to be associated with PGT121 susceptibility are listed in Table 2, ranked by descending PPV. Of the residues previously reported to confer susceptibility to PGT121, 3071, 295 PNGS and 300 PNGS were not statistically associated with susceptibility to PGT121/GS-9722 in this clade B dataset. We identified many previously unreported residues to be significantly associated with susceptibility to PGT121/GS-9722.

TABLE 2
Individual genotypes associated with susceptibility to
PGT121/GS-9722 neutralization among clade B Envs

| | | | Fisher's Exact | Mann-Whitney |
|---|---|---|---|---|
| Virus genotype¹ | PPV | Sensitivity | P value | P value |
| K677 | 78.8 | 31.8 | 0.005 | 0.002 |
| not_W17 | 75.3 | 47.3 | 0.0031 | 0.0001 |
| 332 glycan* | 75.1 | 98.4 | 0.0001 | 0.0001 |
| not_R747 | 74.4 | 51.9 | 0.0023 | 0.0075 |
| insertion_321.01 | 73.8 | 45.7 | 0.0118 | 0.0365 |
| E429 | 71.7 | 80.6 | 0.0001 | 0.0001 |
| Q442 | 70.7 | 50.4 | 0.0423 | 0.0155 |
| T63 | 69.6 | 86.8 | 0.0002 | 0.0009 |
| R335 | 69.3 | 47.3 | 0.1092 | 0.0035 |
| H330* | 68.9 | 87.6 | 0.0003 | 0.0009 |
| i165 | 68.3 | 66.7 | 0.0397 | 0.1486 |
| D325* | 67.3 | 89.1 | 0.0037 | 0.0033 |
| T320 | 66.5 | 86.0 | 0.0266 | 0.0193 |
| L179 | 66.0 | 81.4 | 0.0855 | 0.0123 |
| S393 | 65.2 | 82.9 | 0.1529 | 0.0165 |
| 301 glycan* | 64.5 | 98.4 | 0.0158 | 0.0147 |
| i307* | 64.1 | 91.5 | 0.2443 | 0.5291 |
| 295 glycan* | 63.9 | 76.7 | 0.617 | 0.1188 |
| N300* | 61.9 | 74.4 | 0.7423 | 0.0629 |
| no selection | 62.6 | 100 | na | na |

¹Virus genotype, indicates the presence of specific amino acid residues translated from the HIV envelope gene
*Residue reported in the literature to confer susceptibility to PGT121 neutralization (Julg et al, Sci Transl Med. (2017) 9(408)).

Since an epitope is comprised of more than one residue, combinations of genotypic determinants that were statistically associated with susceptibility to PGT121/GS-9722 were evaluated to see if combining individual genotypic determinants improved the PPV by preferentially enriching true positives over false positives. Consideration was also given to sensitivity since genotypes with low sensitivity will require screening of a larger number of subjects in order to enroll sufficient number of subjects in clinical trials.

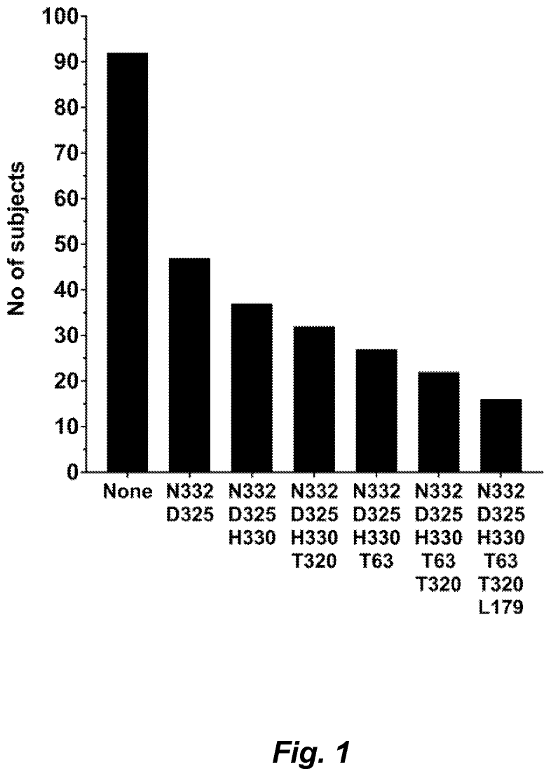

The combination genotypes that provided the highest PPV and sensitivity are listed in Table 3 and displayed in FIG. 1. Several combination genotypes that incorporated previously unreported genotypes associated with susceptibility to PGT121/GS-9722 neutralization provided higher PPV than was achievable using only previously described genotypes. The highest PPV obtained was 98.4% (for viruses containing the amino acids N332 glycan/D325/H330/T63/T320/L179), which represents a 57% increase over the positive predictive value of 62.6% with no genotype selection.

TABLE 3
Individual and combination genotypes associated with susceptibility to
PGT121/GS-9722 neutralization among clade B Envs

| | | | Fisher's Exact | Mann-Whitney |
|---|---|---|---|---|
| Virus genotype¹ | PPV | Sensitivity | P value | P value |
| N332glycan/D325/H330/T63/T320/L179 | 98.4 | 47.3 | 0.0001 | 0.0001 |
| N332glycan/D325/H330/T63/T320 | 93.7 | 57.4 | 0.0001 | 0.0001 |
| N332glycan/D325/H330/T320/L179 | 93.3 | 54.3 | 0.0001 | n.a. |
| N332glycan/D325/H330/T63 | 91.6 | 67.4 | 0.0001 | 0.0001 |
| N332glycan/D325/H330/T320 | 86.1 | 67.4 | 0.0001 | 0.0001 |
| 332PNGS/301PNGS/D325/H330* | 83.9 | 76.7 | 0.0001 | 0.0001 |
| N332glycan/D325/H330* | 83.5 | 78.3 | 0.0001 | 0.0001 |
| N332glycan/D325* | 80.7 | 87.6 | 0.0001 | 0.0001 |
| glycan332 | 75.1 | 98.4 | 0.0001 | 0.0001 |
| glycan301 | 64.5 | 98.4 | 0.0158 | 0.0147 |
| D325 | 67.3 | 89.1 | 0.0037 | 0.0033 |
| H330 | 68.9 | 87.6 | 0.0003 | 0.0009 |
| T63 | 69.6 | 86.8 | 0.0002 | 0.0009 |
| T320 | 66.5 | 86 | 0.0266 | 0.0193 |
| L179 | 66 | 81.4 | 0.0855 | 0.0123 |
| no selection² | 62.6 | 100 | n.a. | n.a. |

¹Virus genotype, indicates the presence of specific amino acid residues translated from the HIV envelope gene
³None, indicates 206 subtype B viruses without selection for specific amino acids in the HIV envelope gene
*indicates genotypes comprised of residues previously reported in the literature to be associated with susceptibility to PGT121. See, e.g., Julg et al, Sci Trans/Med. (2017) 9(408).

The combination genotypes for PGT121/GS-9722 in Table 3 for clade B were used to determine PPV, sensitivity and prevalence for clade A (Table 4) and clade C (Table 5) using neutralization data and corresponding Env sequence for 66 clade A Envs and 258 clade C Envs. The clade A and clade C datasets were publicly-available data obtained from the Los Alamos HIV Sequence Database. The highest PPV obtained for clade A was 93.8% (for viruses containing the amino acids N332glycan/D325/H330/T320/L179), which represents an 88% increase over the positive predictive value of 50% with no genotype selection. The highest PPV obtained for clade C was 89.3% (for viruses containing the amino acids N332glycan/D325/H330/T320/L179), which represents a 53% increase over the positive predictive value of 58.5% with no genotype selection.

TABLE 4
Individual and combination genotypes associated with susceptibility to
PGT121 neutralization among clade A Envs

| Virus genotype¹ | PPV | Sensitivity | Prevalence |
|---|---|---|---|
| N332glycan/D325/H330/T63/T320/L179 | 92.9 | 39.4 | 21.2 |
| N332glycan/D325/H330/T63/T320 | 70.8 | 51.5 | 36.4 |
| N332glycan/D325/H330/T63 | 69.2 | 54.6 | 39.4 |
| N332glycan/D325/H330/T320/L179 | 93.8 | 45.5 | 24.2 |
| N332glycan/D325/H330/T320 | 73.1 | 57.6 | 39.4 |
| N332glycan/D325/H330 | 71.4 | 60.6 | 42.4 |
| N332glycan/D325 | 68.8 | 66.7 | 48.5 |
| glycan332 | 68.4 | 78.8 | 57.6 |
| no selection² | 50 | 100 | 100 |

¹Virus genotype, indicates the presence of specific amino acid residues translated from the HIV envelope gene
²None, indicates 66 clade A viruses without selection for specific amino acids in the HIV envelope gene

TABLE 5
Individual and combination genotypes associated with susceptibility to
PGT121 neutralization among clade C Envs

| Virus genotype¹ | PPV | Sensitivity | Prevalence |
|---|---|---|---|
| N332glycan/D325/H330/T63/T320/L179 | 81.8 | 6.0 | 4.3 |
| N332glycan/D325/H330/T63/T320 | 85.7 | 8.0 | 5.4 |
| N332glycan/D325/H330/T63 | 86.7 | 8.6 | 5.8 |
| N332glycan/D325/H330/T320/L179 | 89.3 | 44.4 | 29.1 |
| N332glycan/D325/H330/T320 | 88.0 | 62.9 | 41.9 |
| N332glycan/D325/H330 | 86.6 | 72.9 | 49.2 |
| N332glycan/D325 | 81.1 | 82.1 | 59.3 |
| glycan332 | 73.7 | 92.7 | 73.6 |
| no selection² | 58.5 | 100 | 100 |

¹Virus genotype, indicates the presence of specific amino acid residues translated from the HIV envelope gene
²None, indicates 258 clade C viruses without selection for specific amino acids in the HIV envelope gene

The prevalence of individual amino acids (T63, L179, T320, D325, H330, N332, NotP333 and S/T334) used in the PGT121/GS-9722 combination genotypes were determined for the clade A, clade B and clade C virus sequences (Table 6). All amino acids show prevalence above 60% in clade B, in clade A except for L179 (51.5%), and in clade C except for T63 (10.1%).

TABLE 6
Prevalence of individual amino acids in clade A,
clade B and clade C viruses

| | Prevalence¹ | | |
|---|---|---|---|
| Position | clade A | clade B | clade C |
| T63 | 84.8 | 78.2 | 10.1 |
| L179 | 51.5 | 77.2 | 63.2 |
| T320 | 89.4 | 81.1 | 86 |
| D325 | 80.3 | 83 | 80.2 |
| H330 | 72.7 | 79.6 | 75.2 |
| N332 | 66.7 | 86.9 | 83.7 |
| NotP333 | 100 | 100 | 100 |
| S/T334 | 62.1 | 84 | 77.6 |

¹Analysis based on the 66 clade A, 206 clade B and 258 clade C viruses from the PGT121/GS-9722 datasets

10-1074 is a broadly neutralizing antibody that targets the V3 glycan region of HIV gp120 and that is related to PGT121/GS-9722. See, e.g., Mouquet, et al., Proc Natl Acad Sci USA. 2012 Nov. 20; 109 (47): E3268-77 and Walker, et al., Nature. 2011 Sep. 22; 477 (7365): 466-70. The combination genotypes for PGT121/GS-9722 in Table 3 were used to determine PPV, sensitivity and prevalence for 10-1074 using neutralization data and corresponding Env sequence for 315 clade B Envs (Table 7). The 315 clade B dataset consisted of 143 clade B Envs isolated from viremic subjects enrolled in Gilead-sponsored clinical trials and 172 clade B Envs from publicly-available data obtained from the Los Alamos HIV Sequence Database. The highest PPV obtained was 100% (for viruses containing the amino acids N332glycan/D325/H330/T63/T320/L179), which represents a 61% increase over the positive predictive value of 62.2% with no genotype selection.

TABLE 7
Individual and combination genotypes associated with susceptibility to
10-1074 neutralization among clade B Envs

| Virus genotype¹ | PPV | Sensitivity | Prevalence |
|---|---|---|---|
| N332glycan/D325/H330/T63/T320/L179 | 100.0 | 38.8 | 24.1 |
| N332glycan/D325/H330/T63/T320 | 99.0 | 51.5 | 32.4 |
| N332glycan/D325/H330/T63 | 98.5 | 65.8 | 41.6 |
| N332glycan/D325/H330/T320/L179 | 96.8 | 46.4 | 29.8 |
| N332glycan/D325/H330/T320 | 94.4 | 59.7 | 39.4 |
| N332glycan/D325/H330* | 93.6 | 75.0 | 49.8 |
| N332glycan/D325 | 92.2 | 84.7 | 57.1 |
| glycan332 | 86.9 | 98.0 | 70.2 |
| no selection² | 62.2 | 100 | 100 |

¹Virus genotype, indicates the presence of specific amino acid residues translated from the HIV envelope gene
²None, indicates 315 clade B viruses without selection for specific amino acids in the HIV envelope gene

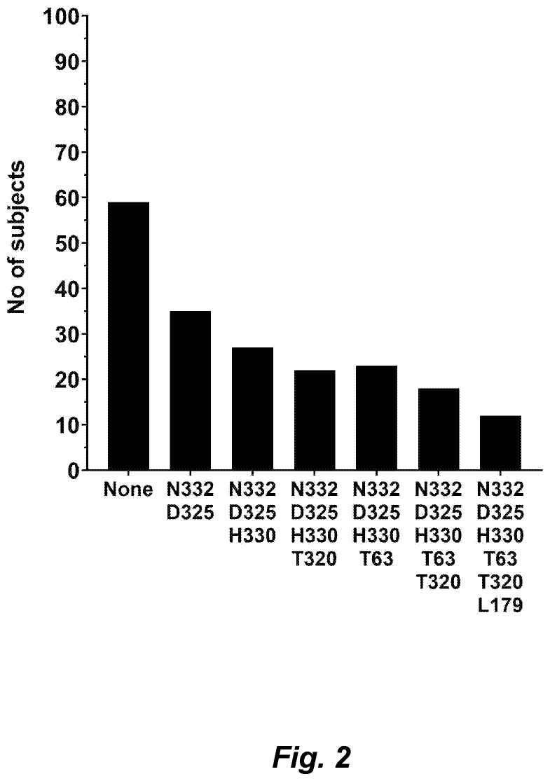

Subsequently, the highest scoring genotypic algorithms (Table 3) were applied to analyze pre-ART plasma samples from HIV infected individuals from the Zurich Primary HIV Infection Cohort Study (ZPHI) to predict whether they would be sensitive to GS-9722 treatment. A total of 92 individual plasma samples were analyzed in a NGS assay of the HIV envelope gene (GenoSure HIV Envelope RNA Assay, Monogram Biosciences, South San Francisco, CA). Subjects were characterized as positive for a given genotype if the derived virus sequences contained the amino acids specified by the algorithm without sequence variability (zero sequence variability on the specified positions). With these criteria, 47/92, 37/92, 32/92, 27/92, 22/92, and 16/92 subjects were predicted to be sensitivity to GS-9722 (FIG. 1) with corresponding positive predictive values of 80.7%, 83.5%, 86.1%, 91.6%, 93.7%, and 98.4%, respectively (Table 3). For clade B infected subjects (59 of the 92 subjects), 35/59, 27/59, 22/59, 23/59, 18/59, and 12/59 were predicted to be sensitivity to GS-9722 (FIG. 2) with corresponding positive predictive values of 80.7%, 83.5%, 86.1%, 91.6%, 93.7%, and 98.4%, respectively (Table 3).

The 100% conservation (zero sequence variability on the specified positions) of the individual amino acids (T63, L179, T320, D325, H330, N332, NotP333 and S/T334) used in the combination genotypes for GS-9722 sensitivity prediction was determined for pre-ART plasma samples for all subjects (n=92) and for the subset of subjects infected with clade B (n=59), (Table 8).

TABLE 8
100% conservation of individual amino acids in ZPHI subjects

| | 100% conservation (% of subjects) | |
|---|---|---|
| Position | All subjects | Clade B infected subjects |
| T63 | 64 | 75 |
| L179 | 59 | 58 |
| T320 | 86 | 85 |
| D325 | 70 | 73 |
| H330 | 65 | 71 |
| N332 | 76 | 85 |
| NotP333 | 100 | 100 |
| S/T334 | 74 | 85 |

¹Analysis based on 92 (all subjects) and 59 (clade B subjects) pre-ART plasma samples from ZPHI individuals

To confirm the genotypic prediction for sensitivity to GS-9722, virus swarms from pre-ART plasma samples from ZPHI were cloned and evaluated in a GS-9722 neutralization assay (PhenoSense HIV Entry Assay, Monogram Biosciences, South San Francisco, CA). Virus was derived from 29 clade B samples with positive predictive values of 80.7% or higher. The derived viruses were characterized as GS-9722 sensitive when IC50s were 1 μg/ml or below. With these criteria, 25/29, 20/22, 16/18, 18/20, 14/16, and 10/11 viruses were confirmed to be sensitivity to GS-9722 (FIG. 2) with corresponding positive predictive values of 80.7%, 83.5%, 86.1%, 91.6%, 93.7%, and 98.4%, respectively (Table 3).

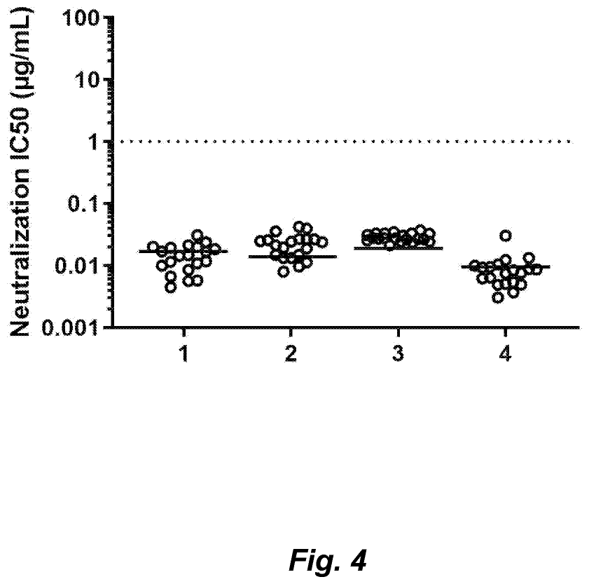

To further confirm the genotypic prediction and phenotypic sensitivity to GS-9722, 20 individual viruses from 4 virus swarms from pre-ART plasma samples from ZPHI were subcloned and evaluated in a GS-9722 neutralization assay (PhenoSense HIV Entry Assay, Monogram Biosciences, South San Francisco, CA). All individual viruses were sensitive to GS-9722 with comparable IC50s to the swarm virus (FIG. 4).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating or preventing HIV in a human subject in need thereof, the method comprising:
a) Identifying a human subject who is infected with an HIV or a population of HIV expressing a gp120 comprising the following amino acid residues: N332glycan, D325, and one or more amino acid residues selected from the group consisting of T63, L179, T320, and H330, wherein the amino acid positions are with reference to SEQ ID NO: 4; and
b) Administering to the subject an effective amount of an antibody or antigen-binding fragment thereof that competes with or comprises VH and VL regions that bind to an epitope of gp120 within the third variable loop (V3) and/or high mannose patch comprising a N332 oligomannose glycan; and
c) Administering to the subject a second antibody or antigen binding fragment thereof that binds to an epitope or region of gp120 selected from the group consisting of:
i. second variable loop (V2) and/or Env trimer apex;
ii. CD4 binding site (CD4bs);
iii. gp120/gp41 interface; or
iv. silent face of gp120.

2. The method of claim 1, comprising identifying a subject infected with an HIV or a population of HIV expressing a gp120 comprising the following amino acid residues:
i. N332glycan, D325 and T63;
ii. N332glycan, D325 and L179;
iii. N332glycan, D325 and T320;
iv. N332glycan, D325 and H330;
v. N332glycan, D325, T63 and L179;
vi. N332glycan, D325, T63 and T320;
vii. N332glycan, D325, T63 and H330;
viii. N332glycan, D325, L179 and T320;
ix. N332glycan, D325, L179 and H330;
x. N332glycan, D325, T320 and H330;
xi. N332glycan, D325, T63, T320 and H330;
xii. N332glycan, D325, T63, L179 and T320;
xiii. N332glycan, D325, T63, L179 and H330;
xiv. N332glycan, D325, L179, T320 and H330; or
xv. N332glycan, D325, T63, L179, T320 and H330.

3. The method of claim 1, comprising identifying a subject infected with an HIV or a population of HIV expressing a gp120 comprising the following amino acid residues:
i. N332glycan, D325 and T63;
ii. N332glycan, D325 and L179;
iii. N332glycan, D325 and T320; or
iv. N332glycan, D325 and H330.

4. The method of claim 1, comprising identifying a subject infected with an HIV or a population of HIV expressing a gp120 comprising the following amino acid residues:
i. N332glycan, D325, T63 and L179;
ii. N332glycan, D325, T63 and T320;
iii. N332glycan, D325, T63 and H330;
iv. N332glycan, D325, L179 and T320;
v. N332glycan, D325, L179 and H330; or
vi. N332glycan, D325, T320 and H330.

5. The method of claim 1, comprising identifying a subject infected with an HIV or a population of HIV expressing a gp120 comprising the following amino acid residues:
i. N332glycan, D325, L179, T320 and H330;
ii. N332glycan, D325, T63, T320 and H330;
iii. N332glycan, D325, T63, L179 and T320; or
iv. N332glycan, D325, T63, L179 and H330.

6. The method of claim 1, comprising identifying a subject infected with an HIV or a population of HIV expressing a gp120 comprising the following amino acid residues:
i. N332glycan, D325, T63 and H330;
ii. N332glycan, D325, T320 and H330;
iii. N332glycan, D325, L179, T320 and H330; or
iv. N332glycan, D325, T63, L179, T320 and H330.

7. (canceled)

8. The method of claim 1, wherein at least 90%, e.g., at least 91%, at least 92%, at least 93%, at least 94%, at least 95%, at least 96%, at least 97%, at least 98%, at least 99%, or 100%, of the HIV species in the population of HIV comprise the recited amino acid residues.

9. (canceled)

10. The method of claim 1, wherein the antibody or antigen-binding fragment thereof competes with or comprises VH and VL regions from an antibody selected from the group consisting of GS-9722, GS-9721, PGT-121, PGT-121.66, PGT-121.414, PGT-124, PGT-134, GS-2872, 10-1074, 10-1074-J, PGT-122 and PGT-123.

11. The method of claim 1, wherein the antibody comprises an Fc region comprising the following amino acids at the indicated positions (EU index numbering):
i. Tyrosine at position 252, threonine at position 254 and glutamic acid at position 256 (YTE); or
ii. Leucine at position 428 and serine at position 434 (LS).

12. The method of claim 1, wherein the antibody comprises an Fc region comprising the following amino acids at the indicated positions (EU index numbering):
i. Aspartate at position 239 and glutamate at position 332 (DE);
ii. Aspartate at position 239, glutamate at position 332 and leucine at position 330 (DEL);
iii. Aspartate at position 239, glutamate at position 332, alanine at position 236 (DEA); or
iv. Aspartate at position 239, glutamate at position 332, alanine at position 236 and leucine at position 330 (DEAL).

13. The method of claim 1, comprising administering an antigen binding fragment.

14. The method of claim 13, wherein the antigen binding fragment is selected from the group consisting of scFv, Fab, Fab2, Fab′, F(ab′)2, Fv, and a diabody.

15. The method of claim 1, wherein the antibody is a multi-specific antibody.

16. The method of claim 1, wherein the human subject is acutely infected with HIV.

17. The method of claim 16, wherein the antibody is administered to a human subject having an HIV infection of Fiebig stage IV or earlier.

18. The method of claim 16, wherein the antibody is administered to a human subject who has not seroconverted.

19. The method of claim 1, wherein the human subject is recently infected with HIV.

20. The method of claim 19, wherein the antibody is administered to a human subject having an HIV infection of Fiebig stage V or Fiebig stage VI.

21. The method of claim 1, wherein the human subject is chronically infected with HIV.

22. The method of claim 1, wherein the human subject is infected with HIV clade B viruses.

23. The method of claim 22, comprising identifying a subject infected with an HIV or a population of HIV expressing a gp120 comprising the following amino acid residues:
i. N332glycan, D325, T63 and H330;
ii. N332glycan, D325, L179, T320 and H330; or
iii. N332glycan, D325, T63, L179, T320 and H330.

24. The method of claim 1, wherein the human subject is infected with HIV clade A viruses.

25. The method of claim 1, wherein the human subject is infected with HIV clade C viruses.

26. The method of claim 1, further comprising administering to the subject one or more additional therapeutic agents for treating an HIV infection.

27. The method of claim 1, wherein the subject is not receiving antiretroviral therapy (ART) or ART is discontinued prior to administration of the antibody.

28. The method of claim 1, wherein ART is discontinued after one or more administrations of the antibody or antigen-binding fragment thereof.

29. The method of claim 1, further comprising administering one or more antiretroviral therapy (ART) agents to the subject.

30. (canceled)

31. The method of claim 1, wherein the second antibody or antigen-binding fragment thereof binds to an epitope or region of gp 120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01.

32. The method of claim 1, wherein the second antibody or antigen-binding fragment thereof binds to an epitope or region of gp 120 in the CD4 binding site (CD4bs) and competes with or comprises VH and VL regions from an antibody selected from the group consisting of b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N6LS (VRC-HIVMAB091-00-AB), N49-P7, NC-Cow1, IOMA, CH235 and CH235.12, N49P6, N49P7, N49P11, N49P9 and N60P25.

33. The method of claim 1, wherein the second antibody or antigen-binding fragment thereof binds to an epitope or region of gp 120 in the gp120/gp41 interface and competes with or comprises VH and VL regions from an antibody selected from the group consisting of PGT-151, CAP248-2B, 35022, 8ANC195, ACS202, VRC34 and VRC34.01.

34. The method of claim 1, wherein the second antibody or antigen-binding fragment thereof binds to an epitope or region of the gp 120 silent face and competes with or comprises VH and VL regions from antibody VRC-PG05.

35. The method of claim 1, wherein the second antibody or antigen-binding fragment thereof binds to an epitope or region of gp41 in the membrane proximal region (MPER) and competes with or comprises VH and VL regions from an antibody selected from the group consisting of 10E8, 10E8v4, 10E8-5R-100° F., 4E10, DH511.11P, 2F5, 7b2, and LN01.

36. The method of claim 1, wherein the second antibody or antigen-binding fragment thereof binds to an epitope or region of the gp41 fusion peptide and competes with or comprises VH and VL regions from an antibody selected from the group consisting of VRC34 and ACS202.

37. The method of claim 1, further comprising administering to the subject a TLR agonist.

38. The method of claim 37, wherein the TLR agonist is a TLR2 agonist, a TLR3 agonist, a TLR7 agonist, a TLR8 agonist or a TLR9 agonist.

39. The method of claim 38, wherein the TLR7 agonist is selected from the group consisting of vesatolimod, imiquimod, and resiquimod.

40. The method of claim 1, comprising multiple administrations of the antibody or antigen-binding fragment thereof, optionally with a TLR agonist, at predetermined intervals.

41. The method of claim 1, wherein, after one or more administrations of the antibody or antigen-binding fragment thereof, the subject does not exhibit symptoms of HIV or AIDS in the absence of anti-retroviral treatment (ART) for at least 6 months, at least 1 year, at least 2 years, at least 3 years, or more.

42. The method of claim 1, wherein, after one or more administrations of the antibody, the subject has a viral load copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

43. A method of identifying a human subject infected with an HIV or a population of HIV sensitive to an antibody or antigen-binding fragment thereof that competes with or comprises VH and VL regions that bind to an epitope of gp120 within the third variable loop (V3) and/or high mannose patch comprising a N332 oligomannose glycan, the method comprising identifying in a biological sample from the human subject an HIV expressing a gp120 comprising the following amino acid residues: N332glycan, D325, and one or more amino acid residues selected from the group consisting of T63, L179, T320, and H330, wherein the amino acid positions are with reference to SEQ ID NO: 4.

44-62. (canceled)

63. The method of claim 1, wherein the gp120 amino acids are identified in one or more gp120 polypeptide sequences expressed from an HIV or a population of HIV isolated from the subject.

64. The method of claim 1, wherein the gp120 amino acids are identified in one or more gp120 polynucleotide sequences from an HIV or a population of HIV isolated from the subject.

65. The method of claim 64, comprising performing next generation sequencing (NGS) on polynucleotide sequences encoding gp120 from a population of HIV.

66. The method of claim 65, wherein gp120 variants are detected to a frequency level of about 1% of the virus population.

67. The method of claim 1, wherein the gp120 amino acids are identified in one or more biological samples from the subject, wherein the one or more biological sample are obtained from blood, peripheral blood mononuclear cells (PBMCs), serum, plasma, semen or lymph nodes.

68. The method of claim 1, comprising identifying a population of HIV RNA in a serum or plasma sample.

69. The method of claim 1, further comprising the step of obtaining one or more biological samples from the subject.

70. The method of claim 69, wherein two or more biological samples are obtained from the subject.

71. The method of claim 70, wherein the two or more biological samples are obtained from the same tissue or fluid at two or more different time points.

72. The method of claim 70, wherein the two or more biological samples are obtained from different tissues or fluids, or from different anatomical locations.


SEQUENCE LISTING (468 sequences)

SEQ ID NO: 1 - protein, 35 aa, synthetic construct
  REGION 1..35: note = Description of Artificial Sequence: Synthetic polypeptide
CTRPNNNTRKSIHIGPGRAFYTTGEIIGDIRQAHC

SEQ ID NO: 2 - protein, 849 aa, synthetic construct
  REGION 1..849: note = Description of Artificial Sequence: Synthetic polypeptide
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 3 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 4 - protein, 511 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 5 - protein, 475 aa, Human immunodeficiency virus 1
AEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKR

SEQ ID NO: 6 - protein, 481 aa, Human immunodeficiency virus 1
TEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 7 - protein, 5 aa, synthetic construct
  REGION 1..5: note = Description of Artificial Sequence: Synthetic peptide
DSYWS

SEQ ID NO: 8 - protein, 16 aa, synthetic construct
  REGION 1..16: note = Description of Artificial Sequence: Synthetic peptide
YVHKSGDTNYSPSLKS

SEQ ID NO: 9 - protein, 24 aa, synthetic construct
  REGION 1..24: note = Description of Artificial Sequence: Synthetic peptide
TLHGRRIYGIVAFNEWFTYFYMDV

SEQ ID NO: 10 - protein, 11 aa, synthetic construct
  REGION 1..11: note = Description of Artificial Sequence: Synthetic peptide
GEKSLGSRAVQ

SEQ ID NO: 11 - protein, 7 aa, synthetic construct
  REGION 1..7: note = Description of Artificial Sequence: Synthetic peptide
NNQDRPS

SEQ ID NO: 12 - protein, 12 aa, synthetic construct
  REGION 1..12: note = Description of Artificial Sequence: Synthetic peptide
HIWDSRVPTKWV

SEQ ID NO: 13 - protein, 16 aa, synthetic construct
  REGION 1..16: note = Description of Artificial Sequence: Synthetic peptide
YVHKSGDTNYNPSLKS

SEQ ID NO: 14 - protein, 5 aa, synthetic construct
  REGION 1..5: note = Description of Artificial Sequence: Synthetic peptide
NYYWT

SEQ ID NO: 15 - protein, 16 aa, synthetic construct
  REGION 1..16: note = Description of Artificial Sequence: Synthetic peptide
YISDRESATYNPSLNS

SEQ ID NO: 16 - protein, 24 aa, synthetic construct
  REGION 1..24: note = Description of Artificial Sequence: Synthetic peptide
ARRGQRIYGVVSFGEFFYYYSMDV

SEQ ID NO: 17 - protein, 11 aa, synthetic construct
  REGION 1..11: note = Description of Artificial Sequence: Synthetic peptide
GRQALGSRAVQ

SEQ ID NO: 18 - protein, 12 aa, synthetic construct
  REGION 1..12: note = Description of Artificial Sequence: Synthetic peptide
HMWDSRSGFSWS

SEQ ID NO: 19 - protein, 16 aa, synthetic construct
  REGION 1..16: note = Description of Artificial Sequence: Synthetic peptide
YISDRETTTYNPSLNS

SEQ ID NO: 20 - protein, 24 aa, synthetic construct
  REGION 1..24: note = Description of Artificial Sequence: Synthetic peptide
ARRGQRIYGVVSFGEFFYYYYMDV

SEQ ID NO: 21 - protein, 5 aa, synthetic construct
  REGION 1..5: note = Description of Artificial Sequence: Synthetic peptide
GRFWS

SEQ ID NO: 22 - protein, 16 aa, synthetic construct
  REGION 1..16: note = Description of Artificial Sequence: Synthetic peptide
YFSDTDRSEYNPSLRS

SEQ ID NO: 23 - protein, 24 aa, synthetic construct
  REGION 1..24: note = Description of Artificial Sequence: Synthetic peptide
AQQGKRIYGIVSFGEFFYYYYMDA

SEQ ID NO: 24 - protein, 11 aa, synthetic construct
  REGION 1..11: note = Description of Artificial Sequence: Synthetic peptide
GERSRGSRAVQ

SEQ ID NO: 25 - protein, 7 aa, synthetic construct
  REGION 1..7: note = Description of Artificial Sequence: Synthetic peptide
NNQDRPA

SEQ ID NO: 26 - protein, 12 aa, synthetic construct
  REGION 1..12: note = Description of Artificial Sequence: Synthetic peptide
HYWDSRSPISWI

SEQ ID NO: 27 - protein, 24 aa, synthetic construct
  REGION 1..24: note = Description of Artificial Sequence: Synthetic peptide
AQQGKRIYGIVSFGELFYYYYMDA

SEQ ID NO: 28 - protein, 5 aa, synthetic construct
  REGION 1..5: note = Description of Artificial Sequence: Synthetic peptide
DNYWS

SEQ ID NO: 29 - protein, 16 aa, synthetic construct
  REGION 1..16: note = Description of Artificial Sequence: Synthetic peptide
YVHDSGDTNYNPSLKS

SEQ ID NO: 30 - protein, 24 aa, synthetic construct
  REGION 1..24: note = Description of Artificial Sequence: Synthetic peptide
TKHGRRIYGVVAFKEWFTYFYMDV

SEQ ID NO: 31 - protein, 11 aa, synthetic construct
  REGION 1..11: note = Description of Artificial Sequence: Synthetic peptide
GEESLGSRSVI

SEQ ID NO: 32 - protein, 7 aa, synthetic construct
  REGION 1..7: note = Description of Artificial Sequence: Synthetic peptide
NNNDRPS

SEQ ID NO: 33 - protein, 12 aa, synthetic construct
  REGION 1..12: note = Description of Artificial Sequence: Synthetic peptide
HIWDSRRPTNWV

SEQ ID NO: 34 - protein, 5 aa, synthetic construct
  REGION 1..5: note = Description of Artificial Sequence: Synthetic peptide
DAYWS

SEQ ID NO: 35 - protein, 16 aa, synthetic construct
  REGION 1..16: note = Description of Artificial Sequence: Synthetic peptide
YVHHSGDTNYNPSLKR

SEQ ID NO: 36 - protein, 24 aa, synthetic construct
  REGION 1..24: note = Description of Artificial Sequence: Synthetic peptide
ALHGKRIYGIVALGELFTYFYMDV

SEQ ID NO: 37 - protein, 11 aa, synthetic construct
  REGION 1..11: note = Description of Artificial Sequence: Synthetic peptide
GKESIGSRAVQ

SEQ ID NO: 38 - protein, 12 aa, synthetic construct
  REGION 1..12: note = Description of Artificial Sequence: Synthetic peptide
HIYDARGGTNWV

SEQ ID NO: 39 - protein, 7 aa, synthetic construct
  REGION 1..7: note = Description of Artificial Sequence: Synthetic peptide
ACTYFWG

SEQ ID NO: 40 - protein, 22 aa, synthetic construct
  REGION 1..22: note = Description of Artificial Sequence: Synthetic peptide
SLSHCQSFWGSGWTFHNPSLKS

SEQ ID NO: 41 - protein, 19 aa, synthetic construct
  REGION 1..19: note = Description of Artificial Sequence: Synthetic peptide
FDGEVLVYNHWPKPAWVDL

SEQ ID NO: 42 - protein, 9 aa, synthetic construct
  REGION 1..9: note = Description of Artificial Sequence: Synthetic peptide
NGTATNFVS

SEQ ID NO: 43 - protein, 7 aa, synthetic construct
  REGION 1..7: note = Description of Artificial Sequence: Synthetic peptide
GVDKRPP

SEQ ID NO: 44 - protein, 10 aa, synthetic construct
  REGION 1..10: note = Description of Artificial Sequence: Synthetic peptide
GSLVGNWDVI

SEQ ID NO: 45 - protein, 7 aa, synthetic construct
  REGION 1..7: note = Description of Artificial Sequence: Synthetic peptide
ACDYFWG

SEQ ID NO: 46 - protein, 22 aa, synthetic construct
  REGION 1..22: note = Description of Artificial Sequence: Synthetic peptide
GLSHCAGYYNTGWTYHNPSLKS

SEQ ID NO: 47 - protein, 19 aa, synthetic construct
  REGION 1..19: note = Description of Artificial Sequence: Synthetic peptide
FDGEVLVYHDWPKPAWVDL

SEQ ID NO: 48 - protein, 9 aa, synthetic construct
  REGION 1..9: note = Description of Artificial Sequence: Synthetic peptide
TGTSNRFVS

SEQ ID NO: 49 - protein, 7 aa, synthetic construct
  REGION 1..7: note = Description of Artificial Sequence: Synthetic peptide
GVNKRPS

SEQ ID NO: 50 - protein, 10 aa, synthetic construct
  REGION 1..10: note = Description of Artificial Sequence: Synthetic peptide
SSLVGNWDVI

SEQ ID NO: 51 - protein, 22 aa, synthetic construct
  REGION 1..22: note = Description of Artificial Sequence: Synthetic peptide
SLSHCAGYYNSGWTYHNPSLKS

SEQ ID NO: 52 - protein, 19 aa, synthetic construct
  REGION 1..19: note = Description of Artificial Sequence: Synthetic peptide
FGGDVLVYHDWPKPAWVDL

SEQ ID NO: 53 - protein, 9 aa, synthetic construct
  REGION 1..9: note = Description of Artificial Sequence: Synthetic peptide
TGNINNFVS

SEQ ID NO: 54 - protein, 10 aa, synthetic construct
  REGION 1..10: note = Description of Artificial Sequence: Synthetic peptide
GSLAGNWDVV

SEQ ID NO: 55 - protein, 7 aa, synthetic construct
  REGION 1..7: note = Description of Artificial Sequence: Synthetic peptide
ACNSFWG

SEQ ID NO: 56 - protein, 22 aa, synthetic construct
  REGION 1..22: note = Description of Artificial Sequence: Synthetic peptide
SLSHCASYWNRGWTYHNPSLKS

SEQ ID NO: 57 - protein, 19 aa, synthetic construct
  REGION 1..19: note = Description of Artificial Sequence: Synthetic peptide
FGGEVLRYTDWPKPAWVDL

SEQ ID NO: 58 - protein, 9 aa, synthetic construct
  REGION 1..9: note = Description of Artificial Sequence: Synthetic peptide
TGTSNNFVS

SEQ ID NO: 59 - protein, 7 aa, synthetic construct
  REGION 1..7: note = Description of Artificial Sequence: Synthetic peptide
DVNKRPS

SEQ ID NO: 60 - protein, 20 aa, synthetic construct
  REGION 1..20: note = Description of Artificial Sequence: Synthetic peptide
ARHRHHDVFMLVPIAGWFDV

SEQ ID NO: 61 - protein, 7 aa, synthetic construct
  REGION 1..7: note = Description of Artificial Sequence: Synthetic peptide
GCDYFWG

SEQ ID NO: 62 - protein, 7 aa, synthetic construct
  REGION 1..7: note = Description of Artificial Sequence: Synthetic peptide
ETYSKIA

SEQ ID NO: 63 - protein, 19 aa, synthetic construct
  REGION 1..19: note = Description of Artificial Sequence: Synthetic peptide
FDGEVLVYNDWPKPAWVDL

SEQ ID NO: 64 - protein, 7 aa, synthetic construct
  REGION 1..7: note = Description of Artificial Sequence: Synthetic peptide
TGHYYWG

SEQ ID NO: 65 - protein, 16 aa, synthetic construct
  REGION 1..16: note = Description of Artificial Sequence: Synthetic peptide
HIHYTTAVLHNPSLKS

SEQ ID NO: 66 - protein, 19 aa, synthetic construct
  REGION 1..19: note = Description of Artificial Sequence: Synthetic peptide
SGGDILYYYEWQKPHWFSP

SEQ ID NO: 67 - protein, 14 aa, synthetic construct
  REGION 1..14: note = Description of Artificial Sequence: Synthetic peptide
NGTSSDIGGWNFVS

SEQ ID NO: 68 - protein, 7 aa, synthetic construct
  REGION 1..7: note = Description of Artificial Sequence: Synthetic peptide
EVNKRPS

SEQ ID NO: 69 - protein, 10 aa, synthetic construct
  REGION 1..10: note = Description of Artificial Sequence: Synthetic peptide
SSLFGRWDVV

SEQ ID NO: 70 - protein, 12 aa, synthetic construct
  REGION 1..12: note = Description of Artificial Sequence: Synthetic peptide
GTDWGENDFHYG

SEQ ID NO: 71 - protein, 17 aa, synthetic construct
  REGION 1..17: note = Description of Artificial Sequence: Synthetic peptide
SIHWRGRTTHYKTSFRS

SEQ ID NO: 72 - protein, 18 aa, synthetic construct
  REGION 1..18: note = Description of Artificial Sequence: Synthetic peptide
HKYHDIFRVVPVAGWFDP

SEQ ID NO: 73 - protein, 11 aa, synthetic construct
  REGION 1..11: note = Description of Artificial Sequence: Synthetic peptide
RASQNVKNNLA

SEQ ID NO: 74 - protein, 7 aa, synthetic construct
  REGION 1..7: note = Description of Artificial Sequence: Synthetic peptide
DASSRAG

SEQ ID NO: 75 - protein, 9 aa, synthetic construct
  REGION 1..9: note = Description of Artificial Sequence: Synthetic peptide
QQYEEWPRT

SEQ ID NO: 76 - protein, 12 aa, synthetic construct
  REGION 1..12: note = Description of Artificial Sequence: Synthetic peptide
GGEWGDSDYHWG

SEQ ID NO: 77 - protein, 16 aa, synthetic construct
  REGION 1..16: note = Description of Artificial Sequence: Synthetic peptide
SIHWRGTTHYNAPFRG

SEQ ID NO: 78 - protein, 18 aa, synthetic construct
  REGION 1..18: note = Description of Artificial Sequence: Synthetic peptide
HKYHDIVMVVPIAGWFDP

SEQ ID NO: 79 - protein, 11 aa, synthetic construct
  REGION 1..11: note = Description of Artificial Sequence: Synthetic peptide
RASQSVKNNLA

SEQ ID NO: 80 - protein, 7 aa, synthetic construct
  REGION 1..7: note = Description of Artificial Sequence: Synthetic peptide
DTSSRAS

SEQ ID NO: 81 - protein, 12 aa, synthetic construct
  REGION 1..12: note = Description of Artificial Sequence: Synthetic peptide
GGEWGDKDYHWG

SEQ ID NO: 82 - protein, 16 aa, synthetic construct
  REGION 1..16: note = Description of Artificial Sequence: Synthetic peptide
SIHWRGTTHYKESLRR

SEQ ID NO: 83 - protein, 18 aa, synthetic construct
  REGION 1..18: note = Description of Artificial Sequence: Synthetic peptide
HRHHDVFMLVPIAGWFDV

SEQ ID NO: 84 - protein, 11 aa, synthetic construct
  REGION 1..11: note = Description of Artificial Sequence: Synthetic peptide
RASQNINKNLA

SEQ ID NO: 85 - protein, 6 aa, synthetic construct
  REGION 1..6: note = Description of Artificial Sequence: Synthetic peptide
SDHSWT

SEQ ID NO: 86 - protein, 16 aa, synthetic construct
  REGION 1..16: note = Description of Artificial Sequence: Synthetic peptide
DIHYNGATTYNPSLRS

SEQ ID NO: 87 - protein, 21 aa, synthetic construct
  REGION 1..21: note = Description of Artificial Sequence: Synthetic peptide
NAIRIYGVVALGEWFHYGMDV

SEQ ID NO: 88 - protein, 11 aa, synthetic construct
  REGION 1..11: note = Description of Artificial Sequence: Synthetic peptide
SGAPLTSRFTY

SEQ ID NO: 89 - protein, 7 aa, synthetic construct
  REGION 1..7: note = Description of Artificial Sequence: Synthetic peptide
RSSQRSS

SEQ ID NO: 90 - protein, 11 aa, synthetic construct
  REGION 1..11: note = Description of Artificial Sequence: Synthetic peptide
QSSDTSDSYKM

SEQ ID NO: 91 - protein, 16 aa, synthetic construct
  REGION 1..16: note = Description of Artificial Sequence: Synthetic peptide
DVHYNGDNTYNPSLRG

SEQ ID NO: 92 - protein, 21 aa, synthetic construct
  REGION 1..21: note = Description of Artificial Sequence: Synthetic peptide
NVIRVFGVISLGEWFHYGMDV

SEQ ID NO: 93 - protein, 11 aa, synthetic construct
  REGION 1..11: note = Description of Artificial Sequence: Synthetic peptide
SGPPLASRYTY

SEQ ID NO: 94 - protein, 7 aa, synthetic construct
  REGION 1..7: note = Description of Artificial Sequence: Synthetic peptide
RDRQFPS

SEQ ID NO: 95 - protein, 11 aa, synthetic construct
  REGION 1..11: note = Description of Artificial Sequence: Synthetic peptide
QSSDINDSYKM

SEQ ID NO: 96 - protein, 16 aa, synthetic construct
  REGION 1..16: note = Description of Artificial Sequence: Synthetic peptide
DVHYNGDTTYNPSLRG

SEQ ID NO: 97 - protein, 11 aa, synthetic construct
  REGION 1..11: note = Description of Artificial Sequence: Synthetic peptide
SGAALTSRFTY

SEQ ID NO: 98 - protein, 7 aa, synthetic construct
  REGION 1..7: note = Description of Artificial Sequence: Synthetic peptide
RTSQRSS

SEQ ID NO: 99 - protein, 16 aa, synthetic construct
  REGION 1..16: note = Description of Artificial Sequence: Synthetic peptide
DIHYGGDITYNPSLRS

SEQ ID NO: 100 - protein, 21 aa, synthetic construct
  REGION 1..21: note = Description of Artificial Sequence: Synthetic peptide
NVIRVFGVIALGEWFHYGMDV

SEQ ID NO: 101 - protein, 11 aa, synthetic construct
  REGION 1..11: note = Description of Artificial Sequence: Synthetic peptide
SGPPLASRYCY

SEQ ID NO: 102 - protein, 7 aa, synthetic construct
  REGION 1..7: note = Description of Artificial Sequence: Synthetic peptide
RDRQFSS

SEQ ID NO: 103 - protein, 11 aa, synthetic construct
  REGION 1..11: note = Description of Artificial Sequence: Synthetic peptide
QSSDTSDSFKM

SEQ ID NO: 104 - protein, 11 aa, synthetic construct
  REGION 1..11: note = Description of Artificial Sequence: Synthetic peptide
SGPPLATRYCY

SEQ ID NO: 105 - protein, 16 aa, synthetic construct
  REGION 1..16: note = Description of Artificial Sequence: Synthetic peptide
DIHYNGDKTYNPSLRG

SEQ ID NO: 106 - protein, 15 aa, synthetic construct
  REGION 1..15: note = Description of Artificial Sequence: Synthetic peptide
SLSHCAGYYNSGWTY

SEQ ID NO: 107 - protein, 11 aa, synthetic construct
  REGION 1..11: note = Description of Artificial Sequence: Synthetic peptide
QSSDNSDSFKM

SEQ ID NO: 108 - protein, 5 aa, synthetic construct
  REGION 1..5: note = Description of Artificial Sequence: Synthetic peptide
DYAMA

SEQ ID NO: 109 - protein, 19 aa, synthetic construct
  REGION 1..19: note = Description of Artificial Sequence: Synthetic peptide
FMRGWAYGGSAQFAAFAVG

SEQ ID NO: 110 - protein, 22 aa, synthetic construct
  REGION 1..22: note = Description of Artificial Sequence: Synthetic peptide
EQRNKDYRYGQEGFGYSYGMDV

SEQ ID NO: 111 - protein, 11 aa, synthetic construct
  REGION 1..11: note = Description of Artificial Sequence: Synthetic peptide
RASHFIANYVN

SEQ ID NO: 112 - protein, 7 aa, synthetic construct
  REGION 1..7: note = Description of Artificial Sequence: Synthetic peptide
ESSTLQR

SEQ ID NO: 113 - protein, 9 aa, synthetic construct
  REGION 1..9: note = Description of Artificial Sequence: Synthetic peptide
QQSHSPPVT

SEQ ID NO: 114 - protein, 19 aa, synthetic construct
  REGION 1..19: note = Description of Artificial Sequence: Synthetic peptide
FIRGWAYGQAAQYGKSASG

SEQ ID NO: 115 - protein, 22 aa, synthetic construct
  REGION 1..22: note = Description of Artificial Sequence: Synthetic peptide
EQRGGDGRYSGDGFGYPYGMDV

SEQ ID NO: 116 - protein, 7 aa, synthetic construct
  REGION 1..7: note = Description of Artificial Sequence: Synthetic peptide
QSWTLNR

SEQ ID NO: 117 - protein, 9 aa, synthetic construct
  REGION 1..9: note = Description of Artificial Sequence: Synthetic peptide
QQSHSPPLS

SEQ ID NO: 118 - protein, 19 aa, synthetic construct
  REGION 1..19: note = Description of Artificial Sequence: Synthetic peptide
FIRGWAYGQSAQYGKSASG

SEQ ID NO: 119 - protein, 22 aa, synthetic construct
  REGION 1..22: note = Description of Artificial Sequence: Synthetic peptide
EQRGANGRYGGDGFGYSYGMDV

SEQ ID NO: 120 - protein, 7 aa, synthetic construct
  REGION 1..7: note = Description of Artificial Sequence: Synthetic peptide
ESSTLNR

SEQ ID NO: 121 - protein, 9 aa, synthetic construct
  REGION 1..9: note = Description of Artificial Sequence: Synthetic peptide
QQSHSPPVS

SEQ ID NO: 122 - protein, 5 aa, synthetic construct
  REGION 1..5: note = Description of Artificial Sequence: Synthetic peptide
HYGGD

SEQ ID NO: 123 - protein, 6 aa, synthetic construct
  REGION 1..6: note = Description of Artificial Sequence: Synthetic peptide
GASISD

SEQ ID NO: 124 - protein, 9 aa, synthetic construct
  REGION 1..9: note = Description of Artificial Sequence: Synthetic peptide
YVHKSGDTN

SEQ ID NO: 125 - protein, 6 aa, synthetic construct
  REGION 1..6: note = Description of Artificial Sequence: Synthetic peptide
GDSMNN

SEQ ID NO: 126 - protein, 9 aa, synthetic construct
  REGION 1..9: note = Description of Artificial Sequence: Synthetic peptide
YISDRESAT

SEQ ID NO: 127 - protein, 6 aa, synthetic construct
  REGION 1..6: note = Description of Artificial Sequence: Synthetic peptide
GGSISN

SEQ ID NO: 128 - protein, 9 aa, synthetic construct
  REGION 1..9: note = Description of Artificial Sequence: Synthetic peptide
YISDRETTT

SEQ ID NO: 129 - protein, 6 aa, synthetic construct
  REGION 1..6: note = Description of Artificial Sequence: Synthetic peptide
NGSVSG

SEQ ID NO: 130 - protein, 9 aa, synthetic construct
  REGION 1..9: note = Description of Artificial Sequence: Synthetic peptide
YFSDTDRSE

SEQ ID NO: 131 - protein, 6 aa, synthetic construct
  REGION 1..6: note = Description of Artificial Sequence: Synthetic peptide
GTLVRD

SEQ ID NO: 132 - protein, 9 aa, synthetic construct
  REGION 1..9: note = Description of Artificial Sequence: Synthetic peptide
YVHDSGDTN

SEQ ID NO: 133 - protein, 6 aa, synthetic construct
  REGION 1..6: note = Description of Artificial Sequence: Synthetic peptide
GASIND

SEQ ID NO: 134 - protein, 9 aa, synthetic construct
  REGION 1..9: note = Description of Artificial Sequence: Synthetic peptide
YVHHSGDTN

SEQ ID NO: 135 - protein, 8 aa, synthetic construct
  REGION 1..8: note = Description of Artificial Sequence: Synthetic peptide
GESTGACT

SEQ ID NO: 136 - protein, 15 aa, synthetic construct
  REGION 1..15: note = Description of Artificial Sequence: Synthetic peptide
SLSHCQSFWGSGWTF

SEQ ID NO: 137 - protein, 8 aa, synthetic construct
  REGION 1..8: note = Description of Artificial Sequence: Synthetic peptide
GDSTAACD

SEQ ID NO: 138 - protein, 15 aa, synthetic construct
  REGION 1..15: note = Description of Artificial Sequence: Synthetic peptide
GLSHCAGYYNTGWTY

SEQ ID NO: 139 - protein, 8 aa, synthetic construct
  REGION 1..8: note = Description of Artificial Sequence: Synthetic peptide
GDSTAACN

SEQ ID NO: 140 - skipped

SEQ ID NO: 141 - protein, 8 aa, synthetic construct
  REGION 1..8: note = Description of Artificial Sequence: Synthetic peptide
GDSTAGCD

SEQ ID NO: 142 - protein, 8 aa, synthetic construct
  REGION 1..8: note = Description of Artificial Sequence: Synthetic peptide
GESINTGH

SEQ ID NO: 143 - protein, 9 aa, synthetic construct
  REGION 1..9: note = Description of Artificial Sequence: Synthetic peptide
HIHYTTAVL

SEQ ID NO: 144 - protein, 13 aa, synthetic construct
  REGION 1..13: note = Description of Artificial Sequence: Synthetic peptide
GGSMRGTDWGEND

SEQ ID NO: 145 - protein, 10 aa, synthetic construct
  REGION 1..10: note = Description of Artificial Sequence: Synthetic peptide
SIHWRGRTTH

SEQ ID NO: 146 - protein, 13 aa, synthetic construct
  REGION 1..13: note = Description of Artificial Sequence: Synthetic peptide
GGSIRGGEWGDSD

SEQ ID NO: 147 - protein, 9 aa, synthetic construct
  REGION 1..9: note = Description of Artificial Sequence: Synthetic peptide
SIHWRGTTH

SEQ ID NO: 148 - protein, 13 aa, synthetic construct
  REGION 1..13: note = Description of Artificial Sequence: Synthetic peptide
GDSIRGGEWGDKD

SEQ ID NO: 149 - protein, 8 aa, synthetic construct
  REGION 1..8: note = Description of Artificial Sequence: Synthetic peptide
QDSRPSDH

SEQ ID NO: 150 - protein, 5 aa, synthetic construct
  REGION 1..5: note = Description of Artificial Sequence: Synthetic peptide
HYNGA

SEQ ID NO: 151 - protein, 8 aa, synthetic construct
  REGION 1..8: note = Description of Artificial Sequence: Synthetic peptide
NDSRPSDH

SEQ ID NO: 152 - protein, 8 aa, synthetic construct
  REGION 1..8: note = Description of Artificial Sequence: Synthetic peptide
GDSRPSDH

SEQ ID NO: 153 - protein, 5 aa, synthetic construct
  REGION 1..5: note = Description of Artificial Sequence: Synthetic peptide
HYNGD

SEQ ID NO: 154 - protein, 7 aa, synthetic construct
  REGION 1..7: note = Description of Artificial Sequence: Synthetic peptide
GFYFPDY

SEQ ID NO: 155 - protein, 8 aa, synthetic construct
  REGION 1..8: note = Description of Artificial Sequence: Synthetic peptide
RGWAYGGS

SEQ ID NO: 156 - protein, 7 aa, synthetic construct
  REGION 1..7: note = Description of Artificial Sequence: Synthetic peptide
DFYFPDY

SEQ ID NO: 157 - protein, 8 aa, synthetic construct
  REGION 1..8: note = Description of Artificial Sequence: Synthetic peptide
RGWAYGQA

SEQ ID NO: 158 - protein, 7 aa, synthetic construct
  REGION 1..7: note = Description of Artificial Sequence: Synthetic peptide
DFYFPDY

SEQ ID NO: 159 - protein, 8 aa, synthetic construct
  REGION 1..8: note = Description of Artificial Sequence: Synthetic peptide
RGWAYGQS

SEQ ID NO: 160 - protein, 8 aa, synthetic construct
  REGION 1..8: note = Description of Artificial Sequence: Synthetic peptide
GASISDSY

SEQ ID NO: 161 - protein, 7 aa, synthetic construct
  REGION 1..7: note = Description of Artificial Sequence: Synthetic peptide
VHKSGDT

SEQ ID NO: 162 - protein, 26 aa, synthetic construct
  REGION 1..26: note = Description of Artificial Sequence: Synthetic peptide
ARTLHGRRIYGIVAFNEWFTYFYMDV

SEQ ID NO: 163 - protein, 6 aa, synthetic construct
  REGION 1..6: note = Description of Artificial Sequence: Synthetic peptide
SLGSRA

SEQ ID NO: 164 - skipped

SEQ ID NO: 165 - protein, 8 aa, synthetic construct
  REGION 1..8: note = Description of Artificial Sequence: Synthetic peptide
GDSMNNYY

SEQ ID NO: 166 - protein, 7 aa, synthetic construct
  REGION 1..7: note = Description of Artificial Sequence: Synthetic peptide
ISDRESA

SEQ ID NO: 167 - protein, 26 aa, synthetic construct
  REGION 1..26: note = Description of Artificial Sequence: Synthetic peptide
ATARRGQRIYGVVSFGEFFYYYSMDV

SEQ ID NO: 168 - protein, 6 aa, synthetic construct
  REGION 1..6: note = Description of Artificial Sequence: Synthetic peptide
ALGSRA

SEQ ID NO: 169 - protein, 8 aa, synthetic construct
  REGION 1..8: note = Description of Artificial Sequence: Synthetic peptide
GGSISNYY

SEQ ID NO: 170 - protein, 7 aa, synthetic construct
  REGION 1..7: note = Description of Artificial Sequence: Synthetic peptide
ISDRETT

SEQ ID NO: 171 - protein, 26 aa, synthetic construct
  REGION 1..26: note = Description of Artificial Sequence: Synthetic peptide
ATARRGQRIYGVVSFGEFFYYYYMDV

SEQ ID NO: 172 - protein, 8 aa, synthetic construct
  REGION 1..8: note = Description of Artificial Sequence: Synthetic peptide
NGSVSGRF

SEQ ID NO: 173 - protein, 7 aa, synthetic construct
  REGION 1..7: note = Description of Artificial Sequence: Synthetic peptide
FSDTDRS

SEQ ID NO: 174 - protein, 26 aa, synthetic construct
  REGION 1..26: note = Description of Artificial Sequence: Synthetic peptide
ARAQQGKRIYGIVSFGELFYYYYMDA

SEQ ID NO: 175 - protein, 6 aa, synthetic construct
  REGION 1..6: note = Description of Artificial Sequence: Synthetic peptide
SRGSRA

SEQ ID NO: 176 - protein, 26 aa, synthetic construct
  REGION 1..26: note = Description of Artificial Sequence: Synthetic peptide
ARAQQGKRIYGIVSFGEFFYYYYMDA

SEQ ID NO: 177 - protein, 8 aa, synthetic construct
  REGION 1..8: note = Description of Artificial Sequence: Synthetic peptide
GTLVRDNY

SEQ ID NO: 178 - protein, 7 aa, synthetic construct
  REGION 1..7: note = Description of Artificial Sequence: Synthetic peptide
VHDSGDT

SEQ ID NO: 179 - protein, 26 aa, synthetic construct
  REGION 1..26: note = Description of Artificial Sequence: Synthetic peptide
ATTKHGRRIYGVVAFKEWFTYFYMDV

SEQ ID NO: 180 - protein, 6 aa, synthetic construct
  REGION 1..6: note = Description of Artificial Sequence: Synthetic peptide
SIGSRA

SEQ ID NO: 181 - protein, 8 aa, synthetic construct
  REGION 1..8: note = Description of Artificial Sequence: Synthetic peptide
GASINDAY

SEQ ID NO: 182 - protein, 7 aa, synthetic construct
  REGION 1..7: note = Description of Artificial Sequence: Synthetic peptide
VHHSGDT

SEQ ID NO: 183 - protein, 26 aa, synthetic construct
  REGION 1..26: note = Description of Artificial Sequence: Synthetic peptide
ARALHGKRIYGIVALGELFTYFYMDV

SEQ ID NO: 184 - protein, 6 aa, synthetic construct
  REGION 1..6: note = Description of Artificial Sequence: Synthetic peptide
SLGSRS

SEQ ID NO: 185 - skipped

SEQ ID NO: 186 - protein, 10 aa, synthetic construct
  REGION 1..10: note = Description of Artificial Sequence: Synthetic peptide
GESTGACTYF

SEQ ID NO: 187 - protein, 13 aa, synthetic construct
  REGION 1..13: note = Description of Artificial Sequence: Synthetic peptide
LSHCQSFWGSGWT

SEQ ID NO: 188 - protein, 21 aa, synthetic construct
  REGION 1..21: note = Description of Artificial Sequence: Synthetic peptide
ARFDGEVLVYNHWPKPAWVDL

SEQ ID NO: 189 - protein, 4 aa, synthetic construct
  REGION 1..4: note = Description of Artificial Sequence: Synthetic peptide
ATNF

SEQ ID NO: 190 - skipped

SEQ ID NO: 191 - protein, 10 aa, synthetic construct
  REGION 1..10: note = Description of Artificial Sequence: Synthetic peptide
GDSTAACDYF

SEQ ID NO: 192 - protein, 13 aa, synthetic construct
  REGION 1..13: note = Description of Artificial Sequence: Synthetic peptide
LSHCAGYYNTGWT

SEQ ID NO: 193 - protein, 21 aa, synthetic construct
  REGION 1..21: note = Description of Artificial Sequence: Synthetic peptide
ARFDGEVLVYHDWPKPAWVDL

SEQ ID NO: 194 - protein, 4 aa, synthetic construct
  REGION 1..4: note = Description of Artificial Sequence: Synthetic peptide
SNRF

SEQ ID NO: 195 - skipped

SEQ ID NO: 196 - protein, 13 aa, synthetic construct
  REGION 1..13: note = Description of Artificial Sequence: Synthetic peptide
LSHCAGYYNSGWT

SEQ ID NO: 197 - protein, 21 aa, synthetic construct
  REGION 1..21: note = Description of Artificial Sequence: Synthetic peptide
ARFGGDVLVYHDWPKPAWVDL

SEQ ID NO: 198 - protein, 4 aa, synthetic construct
  REGION 1..4: note = Description of Artificial Sequence: Synthetic peptide
INNF

SEQ ID NO: 199 - protein, 10 aa, synthetic construct
  REGION 1..10: note = Description of Artificial Sequence: Synthetic peptide
GDSTAACNSF

SEQ ID NO: 200 - protein, 13 aa, synthetic construct
  REGION 1..13: note = Description of Artificial Sequence: Synthetic peptide
LSHCASYWNRGWT

SEQ ID NO: 201 - protein, 21 aa, synthetic construct
  REGION 1..21: note = Description of Artificial Sequence: Synthetic peptide
ARFGGEVLRYTDWPKPAWVDL

SEQ ID NO: 202 - protein, 4 aa, synthetic construct
  REGION 1..4: note = Description of Artificial Sequence: Synthetic peptide
SNNF

SEQ ID NO: 203 - protein, 10 aa, synthetic construct
  REGION 1..10: note = Description of Artificial Sequence: Synthetic peptide
GDSTAGCDYF

SEQ ID NO: 204 - protein, 13 aa, synthetic construct
  REGION 1..13: note = Description of Artificial Sequence: Synthetic peptide
LSHCAGYYNTGWT

SEQ ID NO: 205 - protein, 21 aa, synthetic construct
  REGION 1..21: note = Description of Artificial Sequence: Synthetic peptide
ARFDGEVLVYNDWPKPAWVDL

SEQ ID NO: 206 - protein, 10 aa, synthetic construct
  REGION 1..10: note = Description of Artificial Sequence: Synthetic peptide
GESINTGHYY

SEQ ID NO: 207 - protein, 7 aa, synthetic construct
  REGION 1..7: note = Description of Artificial Sequence: Synthetic peptide
IHYTTAV

SEQ ID NO: 208 - protein, 21 aa, synthetic construct
  REGION 1..21: note = Description of Artificial Sequence: Synthetic peptide
VRSGGDILYYYEWQKPHWFSP

SEQ ID NO: 209 - protein, 9 aa, synthetic construct
  REGION 1..9: note = Description of Artificial Sequence: Synthetic peptide
SSDIGGWNF

SEQ ID NO: 210 - skipped

SEQ ID NO: 211 - protein, 15 aa, synthetic construct
  REGION 1..15: note = Description of Artificial Sequence: Synthetic peptide
GGSMRGTDWGENDFH

SEQ ID NO: 212 - protein, 8 aa, synthetic construct
  REGION 1..8: note = Description of Artificial Sequence: Synthetic peptide
IHWRGRTT

SEQ ID NO: 213 - protein, 20 aa, synthetic construct
  REGION 1..20: note = Description of Artificial Sequence: Synthetic peptide
ARHKYHDIFRVVPVAGWFDP

SEQ ID NO: 214 - protein, 6 aa, synthetic construct
  REGION 1..6: note = Description of Artificial Sequence: Synthetic peptide
QNVKNN

SEQ ID NO: 215 - skipped

SEQ ID NO: 216 - protein, 15 aa, synthetic construct
  REGION 1..15: note = Description of Artificial Sequence: Synthetic peptide
GGSIRGGEWGDSDYH

SEQ ID NO: 217 - protein, 7 aa, synthetic construct
  REGION 1..7: note = Description of Artificial Sequence: Synthetic peptide
IHWRGTT

SEQ ID NO: 218 - protein, 20 aa, synthetic construct
  REGION 1..20: note = Description of Artificial Sequence: Synthetic peptide
VKHKYHDIVMVVPIAGWFDP

SEQ ID NO: 219 - protein, 6 aa, synthetic construct
  REGION 1..6: note = Description of Artificial Sequence: Synthetic peptide
QSVKNN

SEQ ID NO: 220 - skipped

SEQ ID NO: 221 - protein, 15 aa, synthetic construct
  REGION 1..15: note = Description of Artificial Sequence: Synthetic peptide
GDSIRGGEWGDKDYH

SEQ ID NO: 222 - skipped

SEQ ID NO: 223 - protein, 6 aa, synthetic construct
  REGION 1..6: note = Description of Artificial Sequence: Synthetic peptide
QNINKN

SEQ ID NO: 224 - skipped

SEQ ID NO: 225 - protein, 9 aa, synthetic construct
  REGION 1..9: note = Description of Artificial Sequence: Synthetic peptide
QDSRPSDHS

SEQ ID NO: 226 - protein, 7 aa, synthetic construct
  REGION 1..7: note = Description of Artificial Sequence: Synthetic peptide
IHYNGAT

SEQ ID NO: 227 - protein, 6 aa, synthetic construct
  REGION 1..6: note = Description of Artificial Sequence: Synthetic peptide
PLTSRF

SEQ ID NO: 228 - skipped

SEQ ID NO: 229 - protein, 9 aa, synthetic construct
  REGION 1..9: note = Description of Artificial Sequence: Synthetic peptide
NDSRPSDHS

SEQ ID NO: 230 - protein, 9 aa, synthetic construct
  REGION 1..9: note = Description of Artificial Sequence: Synthetic peptide
GDSRPSDHS

SEQ ID NO: 231 - protein, 7 aa, synthetic construct
  REGION 1..7: note = Description of Artificial Sequence: Synthetic peptide
VHYNGDN

SEQ ID NO: 232 - protein, 6 aa, synthetic construct
  REGION 1..6: note = Description of Artificial Sequence: Synthetic peptide
PLASRY

SEQ ID NO: 233 - skipped

SEQ ID NO: 234 - protein, 7 aa, synthetic construct
  REGION 1..7: note = Description of Artificial Sequence: Synthetic peptide
VHYNGDT

SEQ ID NO: 235 - protein, 6 aa, synthetic construct
  REGION 1..6: note = Description of Artificial Sequence: Synthetic peptide
ALTSRF

SEQ ID NO: 236 - protein, 7 aa, synthetic construct
  REGION 1..7: note = Description of Artificial Sequence: Synthetic peptide
IHYGGDI

SEQ ID NO: 237 - protein, 6 aa, synthetic construct
  REGION 1..6: note = Description of Artificial Sequence: Synthetic peptide
PLATRY

SEQ ID NO: 238 - protein, 7 aa, synthetic construct
  REGION 1..7: note = Description of Artificial Sequence: Synthetic peptide
IHYNGDK

SEQ ID NO: 239 - protein, 8 aa, synthetic construct
  REGION 1..8: note = Description of Artificial Sequence: Synthetic peptide
GFYFPDYA

SEQ ID NO: 240 - protein, 10 aa, synthetic construct
  REGION 1..10: note = Description of Artificial Sequence: Synthetic peptide
MRGWAYGGSA

SEQ ID NO: 241 - protein, 6 aa, synthetic construct
  REGION 1..6: note = Description of Artificial Sequence: Synthetic peptide
HFIANY

SEQ ID NO: 242 - skipped

SEQ ID NO: 243 - protein, 8 aa, synthetic construct
  REGION 1..8: note = Description of Artificial Sequence: Synthetic peptide
DFYFPDYA

SEQ ID NO: 244 - protein, 10 aa, synthetic construct
  REGION 1..10: note = Description of Artificial Sequence: Synthetic peptide
IRGWAYGQAA

SEQ ID NO: 245 - skipped

SEQ ID NO: 246 - protein, 10 aa, synthetic construct
  REGION 1..10: note = Description of Artificial Sequence: Synthetic peptide
IRGWAYGQSA

SEQ ID NO: 247 - skipped

SEQ ID NO: 248 - protein, 10 aa, synthetic construct
  REGION 1..10: note = Description of Artificial Sequence: Synthetic peptide
VSGASISDSY

SEQ ID NO: 249 - protein, 16 aa, synthetic construct
  REGION 1..16: note = Description of Artificial Sequence: Synthetic peptide
VHKSGDTNYSPSLKSR

SEQ ID NO: 250 - protein, 23 aa, synthetic construct
  REGION 1..23: note = Description of Artificial Sequence: Synthetic peptide
TLHGRRIYGIVAFNEWFTYFYMD

SEQ ID NO: 251 - protein, 8 aa, synthetic construct
  REGION 1..8: note = Description of Artificial Sequence: Synthetic peptide
EKSLGSRA

SEQ ID NO: 252 - protein, 12 aa, synthetic construct
  REGION 1..12: note = Description of Artificial Sequence: Synthetic peptide
NNQDRPSGIPER

SEQ ID NO: 253 - protein, 9 aa, synthetic construct
  REGION 1..9: note = Description of Artificial Sequence: Synthetic peptide
WDSRVPTKW

SEQ ID NO: 254 - protein, 16 aa, synthetic construct
  REGION 1..16: note = Description of Artificial Sequence: Synthetic peptide
VHKSGDTNYNPSLKSR

SEQ ID NO: 255 - protein, 10 aa, synthetic construct
  REGION 1..10: note = Description of Artificial Sequence: Synthetic peptide
VSGDSMNNYY

SEQ ID NO: 256 - protein, 16 aa, synthetic construct
  REGION 1..16: note = Description of Artificial Sequence: Synthetic peptide
ISDRESATYNPSLNSR

SEQ ID NO: 257 - protein, 23 aa, synthetic construct
  REGION 1..23: note = Description of Artificial Sequence: Synthetic peptide
ARRGQRIYGVVSFGEFFYYYSMD

SEQ ID NO: 258 - protein, 8 aa, synthetic construct
  REGION 1..8: note = Description of Artificial Sequence: Synthetic peptide
RQALGSRA

SEQ ID NO: 259 - protein, 9 aa, synthetic construct
  REGION 1..9: note = Description of Artificial Sequence: Synthetic peptide
WDSRSGFSW

SEQ ID NO: 260 - protein, 10 aa, synthetic construct
  REGION 1..10: note = Description of Artificial Sequence: Synthetic peptide
VSGGSISNYY

SEQ ID NO: 261 - protein, 16 aa, synthetic construct
  REGION 1..16: note = Description of Artificial Sequence: Synthetic peptide
ISDRETTTYNPSLNSR

SEQ ID NO: 262 - protein, 23 aa, synthetic construct
  REGION 1..23: note = Description of Artificial Sequence: Synthetic peptide
ARRGQRIYGVVSFGEFFYYYYMD

SEQ ID NO: 263 - protein, 10 aa, synthetic construct
  REGION 1..10: note = Description of Artificial Sequence: Synthetic peptide
VSNGSVSGRF

SEQ ID NO: 264 - protein, 16 aa, synthetic construct
  REGION 1..16: note = Description of Artificial Sequence: Synthetic peptide
FSDTDRSEYNPSLRSR

SEQ ID NO: 265 - protein, 23 aa, synthetic construct
  REGION 1..23: note = Description of Artificial Sequence: Synthetic peptide
AQQGKRIYGIVSFGELFYYYYMD

SEQ ID NO: 266 - protein, 8 aa, synthetic construct
  REGION 1..8: note = Description of Artificial Sequence: Synthetic peptide
ERSRGSRA

SEQ ID NO: 267 - protein, 12 aa, synthetic construct
  REGION 1..12: note = Description of Artificial Sequence: Synthetic peptide
NNQDRPAGVSER

SEQ ID NO: 268 - protein, 9 aa, synthetic construct
  REGION 1..9: note = Description of Artificial Sequence: Synthetic peptide
WDSRSPISW

SEQ ID NO: 269 - protein, 10 aa, synthetic construct
  REGION 1..10: note = Description of Artificial Sequence: Synthetic peptide
VSGASINDAY

SEQ ID NO: 270 - protein, 16 aa, synthetic construct
  REGION 1..16: note = Description of Artificial Sequence: Synthetic peptide
VHHSGDTNYNPSLKRR

SEQ ID NO: 271 - protein, 23 aa, synthetic construct
  REGION 1..23: note = Description of Artificial Sequence: Synthetic peptide
ALHGKRIYGIVALGELFTYFYMD

SEQ ID NO: 272 - protein, 8 aa, synthetic construct
  REGION 1..8: note = Description of Artificial Sequence: Synthetic peptide
KESIGSRA

SEQ ID NO: 273 - protein, 12 aa, synthetic construct
  REGION 1..12: note = Description of Artificial Sequence: Synthetic peptide
NNQDRPAGVPER

SEQ ID NO: 274 - protein, 9 aa, synthetic construct
  REGION 1..9: note = Description of Artificial Sequence: Synthetic peptide
YDARGGTNW

SEQ ID NO: 275 - protein, 10 aa, synthetic construct
  REGION 1..10: note = Description of Artificial Sequence: Synthetic peptide
VSGTLVRDNY

SEQ ID NO: 276 - protein, 16 aa, synthetic construct
  REGION 1..16: note = Description of Artificial Sequence: Synthetic peptide
VHDSGDTNYNPSLKSR

SEQ ID NO: 277 - protein, 23 aa, synthetic construct
  REGION 1..23: note = Description of Artificial Sequence: Synthetic peptide
TKHGRRIYGVVAFKEWFTYFYMD

SEQ ID NO: 278 - protein, 8 aa, synthetic construct
  REGION 1..8: note = Description of Artificial Sequence: Synthetic peptide
EESLGSRS

SEQ ID NO: 279 - protein, 12 aa, synthetic construct
  REGION 1..12: note = Description of Artificial Sequence: Synthetic peptide
NNNDRPSGIPDR

SEQ ID NO: 280 - protein, 9 aa, synthetic construct
  REGION 1..9: note = Description of Artificial Sequence: Synthetic peptide
WDSRRPTNW

SEQ ID NO: 281 - protein, 12 aa, synthetic construct
  REGION 1..12: note = Description of Artificial Sequence: Synthetic peptide
VSGESTGACTYF

SEQ ID NO: 282 - protein, 22 aa, synthetic construct
  REGION 1..22: note = Description of Artificial Sequence: Synthetic peptide
LSHCQSFWGSGWTFHNPSLKSR

SEQ ID NO: 283 - protein, 18 aa, synthetic construct
  REGION 1..18: note = Description of Artificial Sequence: Synthetic peptide
FDGEVLVYNHWPKPAWVD

SEQ ID NO: 284 - protein, 6 aa, synthetic construct
  REGION 1..6: note = Description of Artificial Sequence: Synthetic peptide
GTATNF

SEQ ID NO: 285 - protein, 12 aa, synthetic construct
  REGION 1..12: note = Description of Artificial Sequence: Synthetic peptide
GVDKRPPGVPDR

SEQ ID NO: 286 - protein, 7 aa, synthetic construct
  REGION 1..7: note = Description of Artificial Sequence: Synthetic peptide
LVGNWDV

SEQ ID NO: 287 - protein, 12 aa, synthetic construct
  REGION 1..12: note = Description of Artificial Sequence: Synthetic peptide
VSGDSTAACDYF

SEQ ID NO: 288 - protein, 22 aa, synthetic construct
  REGION 1..22: note = Description of Artificial Sequence: Synthetic peptide
LSHCAGYYNTGWTYHNPSLKSR

SEQ ID NO: 289 - protein, 18 aa, synthetic construct
  REGION 1..18: note = Description of Artificial Sequence: Synthetic peptide
FDGEVLVYHDWPKPAWVD

SEQ ID NO: 290 - protein, 6 aa, synthetic construct
  REGION 1..6: note = Description of Artificial Sequence: Synthetic peptide
GTSNRF

SEQ ID NO: 291 - protein, 12 aa, synthetic construct
  REGION 1..12: note = Description of Artificial Sequence: Synthetic peptide
GVNKRPSGVPDR

SEQ ID NO: 292 - protein, 22 aa, synthetic construct
  REGION 1..22: note = Description of Artificial Sequence: Synthetic peptide
LSHCAGYYNSGWTYHNPSLKSR

SEQ ID NO: 293 - protein, 18 aa, synthetic construct
  REGION 1..18: note = Description of Artificial Sequence: Synthetic peptide
FGGDVLVYHDWPKPAWVD

SEQ ID NO: 294 - protein, 6 aa, synthetic construct
  REGION 1..6: note = Description of Artificial Sequence: Synthetic peptide
GNINNF

SEQ ID NO: 295 - protein, 12 aa, synthetic construct
  REGION 1..12: note = Description of Artificial Sequence: Synthetic peptide
GVNKRPSGVPDR

SEQ ID NO: 296 - protein, 7 aa, synthetic construct
  REGION 1..7: note = Description of Artificial Sequence: Synthetic peptide
LAGNWDV

SEQ ID NO: 297 - protein, 12 aa, synthetic construct
  REGION 1..12: note = Description of Artificial Sequence: Synthetic peptide
VSGDSTAACNSF

SEQ ID NO: 298 - protein, 22 aa, synthetic construct
  REGION 1..22: note = Description of Artificial Sequence: Synthetic peptide
LSHCASYWNRGWTYHNPSLKSR

SEQ ID NO: 299 - protein, 18 aa, synthetic construct
  REGION 1..18: note = Description of Artificial Sequence: Synthetic peptide
FGGEVLRYTDWPKPAWVD

SEQ ID NO: 300 - protein, 6 aa, synthetic construct
  REGION 1..6: note = Description of Artificial Sequence: Synthetic peptide
GTSNNF

SEQ ID NO: 301 - protein, 12 aa, synthetic construct
  REGION 1..12: note = Description of Artificial Sequence: Synthetic peptide
DVNKRPSGVPDR

SEQ ID NO: 302 - protein, 12 aa, synthetic construct
  REGION 1..12: note = Description of Artificial Sequence: Synthetic peptide
VSGDSTAGCDYF

SEQ ID NO: 303 - protein, 18 aa, synthetic construct
  REGION 1..18: note = Description of Artificial Sequence: Synthetic peptide
FDGEVLVYNDWPKPAWVD

SEQ ID NO: 304 - protein, 12 aa, synthetic construct
  REGION 1..12: note = Description of Artificial Sequence: Synthetic peptide
VSGESINTGHYY

SEQ ID NO: 305 - protein, 16 aa, synthetic construct
  REGION 1..16: note = Description of Artificial Sequence: Synthetic peptide
IHYTTAVLHNPSLKSR

SEQ ID NO: 306 - protein, 18 aa, synthetic construct
  REGION 1..18: note = Description of Artificial Sequence: Synthetic peptide
SGGDILYYYEWQKPHWFS

SEQ ID NO: 307 - protein, 11 aa, synthetic construct
  REGION 1..11: note = Description of Artificial Sequence: Synthetic peptide
GTSSDIGGWNF

SEQ ID NO: 308 - protein, 12 aa, synthetic construct
  REGION 1..12: note = Description of Artificial Sequence: Synthetic peptide
EVNKRPSGVPGR

SEQ ID NO: 309 - protein, 7 aa, synthetic construct
  REGION 1..7: note = Description of Artificial Sequence: Synthetic peptide
LFGRWDV

SEQ ID NO: 310 - protein, 17 aa, synthetic construct
  REGION 1..17: note = Description of Artificial Sequence: Synthetic peptide
VSGGSMRGTDWGENDFH

SEQ ID NO: 311 - protein, 17 aa, synthetic construct
  REGION 1..17: note = Description of Artificial Sequence: Synthetic peptide
IHWRGRTTHYKTSFRSR

SEQ ID NO: 312 - protein, 17 aa, synthetic construct
  REGION 1..17: note = Description of Artificial Sequence: Synthetic peptide
HKYHDIFRVVPVAGWFD

SEQ ID NO: 313 - protein, 8 aa, synthetic construct
  REGION 1..8: note = Description of Artificial Sequence: Synthetic peptide
ASQNVKNN

SEQ ID NO: 314 - protein, 12 aa, synthetic construct
  REGION 1..12: note = Description of Artificial Sequence: Synthetic peptide
DASSRAGGIPDR

SEQ ID NO: 315 - protein, 6 aa, synthetic construct
  REGION 1..6: note = Description of Artificial Sequence: Synthetic peptide
YEEWPR

SEQ ID NO: 316 - protein, 17 aa, synthetic construct
  REGION 1..17: note = Description of Artificial Sequence: Synthetic peptide
ASGGSIRGGEWGDSDYH

SEQ ID NO: 317 - protein, 16 aa, synthetic construct
  REGION 1..16: note = Description of Artificial Sequence: Synthetic peptide
IHWRGTTHYNAPFRGR

SEQ ID NO: 318 - protein, 17 aa, synthetic construct
  REGION 1..17: note = Description of Artificial Sequence: Synthetic peptide
HKYHDIVMVVPIAGWFD

SEQ ID NO: 319 - protein, 8 aa, synthetic construct
  REGION 1..8: note = Description of Artificial Sequence: Synthetic peptide
ASQSVKNN

SEQ ID NO: 320 - protein, 12 aa, synthetic construct
  REGION 1..12: note = Description of Artificial Sequence: Synthetic peptide
DTSSRASGIPAR

SEQ ID NO: 321 - protein, 17 aa, synthetic construct
  REGION 1..17: note = Description of Artificial Sequence: Synthetic peptide
VSGDSIRGGEWGDKDYH

SEQ ID NO: 322 - protein, 16 aa, synthetic construct
  REGION 1..16: note = Description of Artificial Sequence: Synthetic peptide
IHWRGTTHYKESLRRR

SEQ ID NO: 323 - protein, 17 aa, synthetic construct
  REGION 1..17: note = Description of Artificial Sequence: Synthetic peptide
HRHHDVFMLVPIAGWFD

SEQ ID NO: 324 - protein, 8 aa, synthetic construct
  REGION 1..8: note = Description of Artificial Sequence: Synthetic peptide
ASQNINKN

SEQ ID NO: 325 - protein, 12 aa, synthetic construct
  REGION 1..12: note = Description of Artificial Sequence: Synthetic peptide
ETYSKIAAFPAR

SEQ ID NO: 326 - protein, 11 aa, synthetic construct
  REGION 1..11: note = Description of Artificial Sequence: Synthetic peptide
VSQDSRPSDHS

SEQ ID NO: 327 - protein, 16 aa, synthetic construct
  REGION 1..16: note = Description of Artificial Sequence: Synthetic peptide
IHYNGATTYNPSLRSR

SEQ ID NO: 328 - protein, 20 aa, synthetic construct
  REGION 1..20: note = Description of Artificial Sequence: Synthetic peptide
NAIRIYGVVALGEWFHYGMD

SEQ ID NO: 329 - protein, 8 aa, synthetic construct
  REGION 1..8: note = Description of Artificial Sequence: Synthetic peptide
GAPLTSRF

SEQ ID NO: 330 - protein, 12 aa, synthetic construct
  REGION 1..12: note = Description of Artificial Sequence: Synthetic peptide
RSSQRSSGWSGR

SEQ ID NO: 331 - protein, 8 aa, synthetic construct
  REGION 1..8: note = Description of Artificial Sequence: Synthetic peptide
SDTSDSYK

SEQ ID NO: 332 - protein, 11 aa, synthetic construct
  REGION 1..11: note = Description of Artificial Sequence: Synthetic peptide
VSNDSRPSDHS

SEQ ID NO: 333 - protein, 11 aa, synthetic construct
  REGION 1..11: note = Description of Artificial Sequence: Synthetic peptide
VFGDSRPSDHS

SEQ ID NO: 334 - protein, 16 aa, synthetic construct
  REGION 1..16: note = Description of Artificial Sequence: Synthetic peptide
VHYNGDNTYNPSLRGR

SEQ ID NO: 335 - protein, 20 aa, synthetic construct
  REGION 1..20: note = Description of Artificial Sequence: Synthetic peptide
NVIRVFGVISLGEWFHYGMD

SEQ ID NO: 336 - protein, 8 aa, synthetic construct
  REGION 1..8: note = Description of Artificial Sequence: Synthetic peptide
GPPLASRY

SEQ ID NO: 337 - protein, 12 aa, synthetic construct
  REGION 1..12: note = Description of Artificial Sequence: Synthetic peptide
RDRQFPSGVSGR

SEQ ID NO: 338 - protein, 8 aa, synthetic construct
  REGION 1..8: note = Description of Artificial Sequence: Synthetic peptide
SDTSDSYK

SEQ ID NO: 339 - protein, 16 aa, synthetic construct
  REGION 1..16: note = Description of Artificial Sequence: Synthetic peptide
VHYNGDTTYNPSLRGR

SEQ ID NO: 340 - protein, 8 aa, synthetic construct
  REGION 1..8: note = Description of Artificial Sequence: Synthetic peptide
GAALTSRF

SEQ ID NO: 341 - protein, 12 aa, synthetic construct
  REGION 1..12: note = Description of Artificial Sequence: Synthetic peptide
RTSQRSSGWSGR

SEQ ID NO: 342 - protein, 11 aa, synthetic construct
  REGION 1..11: note = Description of Artificial Sequence: Synthetic peptide
ISGDSRPSDHS

SEQ ID NO: 343 - protein, 16 aa, synthetic construct
  REGION 1..16: note = Description of Artificial Sequence: Synthetic peptide
IHYGGDITYNPSLRSR

SEQ ID NO: 344 - protein, 20 aa, synthetic construct
  REGION 1..20: note = Description of Artificial Sequence: Synthetic peptide
NVIRVFGVIALGEWFHYGMD

SEQ ID NO: 345 - protein, 12 aa, synthetic construct
  REGION 1..12: note = Description of Artificial Sequence: Synthetic peptide
RDRQFSSGMSGR

SEQ ID NO: 346 - protein, 8 aa, synthetic construct
  REGION 1..8: note = Description of Artificial Sequence: Synthetic peptide
SDINDSYK

SEQ ID NO: 347 - protein, 12 aa, synthetic construct
  REGION 1..12: note = Description of Artificial Sequence: Synthetic peptide
RDRQFSSGISGR

SEQ ID NO: 348 - protein, 8 aa, synthetic construct
  REGION 1..8: note = Description of Artificial Sequence: Synthetic peptide
SDTSDSFK

SEQ ID NO: 349 - protein, 8 aa, synthetic construct
  REGION 1..8: note = Description of Artificial Sequence: Synthetic peptide
GPPLATRY

SEQ ID NO: 350 - protein, 12 aa, synthetic construct
  REGION 1..12: note = Description of Artificial Sequence: Synthetic peptide
RDRQFSSGVSGR

SEQ ID NO: 351 - protein, 16 aa, synthetic construct
  REGION 1..16: note = Description of Artificial Sequence: Synthetic peptide
IHYNGDKTYNPSLRGR

SEQ ID NO: 352 - protein, 8 aa, synthetic construct
  REGION 1..8: note = Description of Artificial Sequence: Synthetic peptide
SDNSDSFK

SEQ ID NO: 353 - protein, 10 aa, synthetic construct
  REGION 1..10: note = Description of Artificial Sequence: Synthetic peptide
ASGFYFPDYA

SEQ ID NO: 354 - protein, 19 aa, synthetic construct
  REGION 1..19: note = Description of Artificial Sequence: Synthetic peptide
MRGWAYGGSAQFAAFAVGK

SEQ ID NO: 355 - protein, 21 aa, synthetic construct
  REGION 1..21: note = Description of Artificial Sequence: Synthetic peptide
EQRNKDYRYGQEGFGYSYGMD

SEQ ID NO: 356 - protein, 8 aa, synthetic construct
  REGION 1..8: note = Description of Artificial Sequence: Synthetic peptide
ASHFIANY

SEQ ID NO: 357 - protein, 12 aa, synthetic construct
  REGION 1..12: note = Description of Artificial Sequence: Synthetic peptide
ESSTLQRGVPSR

SEQ ID NO: 358 - protein, 6 aa, synthetic construct
  REGION 1..6: note = Description of Artificial Sequence: Synthetic peptide
SHSPPV

SEQ ID NO: 359 - protein, 10 aa, synthetic construct
  REGION 1..10: note = Description of Artificial Sequence: Synthetic peptide
AFDFYFPDYA

SEQ ID NO: 360 - protein, 19 aa, synthetic construct
  REGION 1..19: note = Description of Artificial Sequence: Synthetic peptide
IRGWAYGQAAQYGKSASGR

SEQ ID NO: 361 - protein, 21 aa, synthetic construct
  REGION 1..21: note = Description of Artificial Sequence: Synthetic peptide
EQRGGDGRYSGDGFGYPYGMD

SEQ ID NO: 362 - protein, 12 aa, synthetic construct
  REGION 1..12: note = Description of Artificial Sequence: Synthetic peptide
QSWTLNRGIPSR

SEQ ID NO: 363 - protein, 6 aa, synthetic construct
  REGION 1..6: note = Description of Artificial Sequence: Synthetic peptide
SHSPPL

SEQ ID NO: 364 - protein, 19 aa, synthetic construct
  REGION 1..19: note = Description of Artificial Sequence: Synthetic peptide
IRGWAYGQSAQYGKSASGR

SEQ ID NO: 365 - protein, 21 aa, synthetic construct
  REGION 1..21: note = Description of Artificial Sequence: Synthetic peptide
EQRGANGRYGGDGFGYSYGMD

SEQ ID NO: 366 - protein, 12 aa, synthetic construct
  REGION 1..12: note = Description of Artificial Sequence: Synthetic peptide
ESSTLNRGVPSR

SEQ ID NO: 367-396 - skipped (30 entries)

SEQ ID NO: 397 - protein, 23 aa, synthetic construct
  REGION 1..23: note = Description of Artificial Sequence: Synthetic peptide
AQQGKRIYGIVSFGEFFYYYYMD

SEQ ID NO: 398-399 - skipped (2 entries)

SEQ ID NO: 400 - protein, 132 aa, synthetic construct
  REGION 1..132: note = Description of Artificial Sequence: Synthetic polypeptide
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 401 - protein, 105 aa, synthetic construct
  REGION 1..105: note = Description of Artificial Sequence: Synthetic polypeptide
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 402 - protein, 132 aa, synthetic construct
  REGION 1..132: note = Description of Artificial Sequence: Synthetic polypeptide
QMQLQESGPGLVKPSETLSLTCSVSGASISDSYWSWIRRSPGKGLEWIGYVHKSGDTNYNPSLKSRVHLSLDTSKNQVSLSLTGVTAADSGKYYCARTLHGRRIYGIVAFNEWFTYFYMDVWGTGTQVTVSS

SEQ ID NO: 403 - protein, 105 aa, synthetic construct
  REGION 1..105: note = Description of Artificial Sequence: Synthetic polypeptide
SDISVAPGETARISCGEKSLGSRAVQWYQHRAGQAPSLIIYNNQDRPSGIPERFSGSPDFRPGTTATLTITSVEAGDEADYYCHIWDSRVPTKWVFGGGTTLTVL

SEQ ID NO: 404 - protein, 105 aa, synthetic construct
  REGION 1..105: note = Description of Artificial Sequence: Synthetic polypeptide
SDISVAPGETARISCGEKSLGSRAVQWYQHRAGQAPSLIIYNNQDRPSGIPERFSGSPDSRPGTTATLTITSVEAGDEADYYCHIWDSRVPTKWVFGGGTTLTVL

SEQ ID NO: 405 - protein, 132 aa, synthetic construct
  REGION 1..132: note = Description of Artificial Sequence: Synthetic polypeptide
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 406 - protein, 108 aa, synthetic construct
  REGION 1..108: note = Description of Artificial Sequence: Synthetic polypeptide
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 407 - protein, 132 aa, synthetic construct
  REGION 1..132: note = Description of Artificial Sequence: Synthetic polypeptide
QVQLQESGPGLVRPSETLSVTCIVSGGSISNYYWTWIRQSPGKGLEWIGYISDRETTTYNPSLNSRAVISRDTSKNQLSLQLRSVTTADTAIYFCATARRGQRIYGVVSFGEFFYYYYMDVWGKGTAVTVSS

SEQ ID NO: 408 - protein, 107 aa, synthetic construct
  REGION 1..107: note = Description of Artificial Sequence: Synthetic polypeptide
SYVSPLSVALGETARISCGRQALGSRAVQWYQHKPGQAPILLIYNNQDRPSGIPERFSGTPDINFGTTATLTISGVEVGDEADYYCHMWDSRSGFSWSFGGATRLTV

SEQ ID NO: 409 - protein, 132 aa, synthetic construct
  REGION 1..132: note = Description of Artificial Sequence: Synthetic polypeptide
QVHLQESGPGLVTPSETLSLTCTVSNGSVSGRFWSWIRQSPGRGLEWIGYFSDTDRSEYNPSLRSRLTLSVDRSKNQLSLRLKSVTAADSATYYCARAQQGKRIYGIVSFGEFFYYYYMDAWGKGTPVTVSS

SEQ ID NO: 410 - protein, 107 aa, synthetic construct
  REGION 1..107: note = Description of Artificial Sequence: Synthetic polypeptide
SLNPLSLAPGATAKIPCGERSRGSRAVQWYQQKPGQAPTLIIYNNQDRPAGVSERFSGNPDVAIGVTATLTISRVEVGDEADYYCHYWDSRSPISWIFGGGTQLTVL

SEQ ID NO: 411 - protein, 132 aa, synthetic construct
  REGION 1..132: note = Description of Artificial Sequence: Synthetic polypeptide
QVHLQESGPGLVTPSETLSLTCTVSNGSVSGRFWSWIRQSPGRGLEWIGYFSDTDRSEYNPSLRSRLTLSVDRSKNQLSLKLKSVTAADSATYYCARAQQGKRIYGIVSFGELFYYYYMDAWGKGTPVTVSS

SEQ ID NO: 412 - protein, 107 aa, synthetic construct
  REGION 1..107: note = Description of Artificial Sequence: Synthetic polypeptide
SLNPLSLAPGATAKIPCGERSRGSRAVQWYQQKPGQAPTLIIYNNQDRPAGVSERFSGNPDVAIGVTATLTISRVEVGDEGDYYCHYWDSRSPISWIFAGGTQLTVL

SEQ ID NO: 413 - protein, 132 aa, synthetic construct
  REGION 1..132: note = Description of Artificial Sequence: Synthetic polypeptide
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSS

SEQ ID NO: 414 - protein, 105 aa, synthetic construct
  REGION 1..105: note = Description of Artificial Sequence: Synthetic polypeptide
TFVSVAPGQTARITCGEESLGSRSVIWYQQRPGQAPSLIIYNNNDRPSGIPDRFSGSPGSTFGTTATLTITSVEAGDEADYYCHIWDSRRPTNWVFGEGTTLIVL

SEQ ID NO: 415 - protein, 132 aa, synthetic construct
  REGION 1..132: note = Description of Artificial Sequence: Synthetic polypeptide
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSS

SEQ ID NO: 416 - protein, 105 aa, synthetic construct
  REGION 1..105: note = Description of Artificial Sequence: Synthetic polypeptide
SSMSVSPGETAKISCGKESIGSRAVQWYQQKPGQPPSLIIYNNQDRPAGVPERFSASPDFRPGTTATLTITNVDAEDEADYYCHIYDARGGTNWVFDRGTTLTVL

SEQ ID NO: 417 - protein, 135 aa, synthetic construct
  REGION 1..135: note = Description of Artificial Sequence: Synthetic polypeptide
QSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVSS

SEQ ID NO: 418 - protein, 105 aa, synthetic construct
  REGION 1..105: note = Description of Artificial Sequence: Synthetic polypeptide
QSALTQPPSASGSPGQSITISCNGTATNFVSWYQQFPDKAPKLIIFGVDKRPPGVPDRFSGSRSGTTASLTVSRLQTDDEAVYYCGSLVGNWDVIFGGGTTLTVL

SEQ ID NO: 419 - protein, 135 aa, synthetic construct
  REGION 1..135: note = Description of Artificial Sequence: Synthetic polypeptide
QPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVSS

SEQ ID NO: 420 - protein, 105 aa, synthetic construct
  REGION 1..105: note = Description of Artificial Sequence: Synthetic polypeptide
QSALTQPPSASGSPGQSISISCTGTSNRFVSWYQQHPGKAPKLVIYGVNKRPSGVPDRFSGSKSGNTASLTVSGLQTDDEAVYYCSSLVGNWDVIFGGGTKLTVL

SEQ ID NO: 421 - protein, 133 aa, synthetic construct [sequence omitted]

SEQ ID NO: 422 - protein, 107 aa, synthetic construct
  REGION 1..107: note = Description of Artificial Sequence: Synthetic polypeptide
EIVMTQSPDTLSVSPGETVTLSCRASQNINKNLAWYQYKPGQSPRLVIFETYSKIAAFPARFVASGSGTEFTLTINNMQSEDVAVYYCQQYEEWPRTFGQGTKVDIK

SEQ ID NO: 423 - protein, 134 aa, synthetic construct
  REGION 1..134: note = Description of Artificial Sequence: Synthetic polypeptide
QLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSS

SEQ ID NO: 424 - protein, 107 aa, synthetic construct
  REGION 1..107: note = Description of Artificial Sequence: Synthetic polypeptide
EIVMTQSPPTLSVSPGETATLSCRASQNVKNNLAWYQLKPGQAPRLLIFDASSRAGGIPDRFSGSGYGTDFTLTVNSVQSEDFGDYFCQQYEEWPRTFGQGTKVDIK

SEQ ID NO: 425 - protein, 133 aa, synthetic construct
  REGION 1..133: note = Description of Artificial Sequence: Synthetic polypeptide
EVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSS

SEQ ID NO: 426 - protein, 107 aa, synthetic construct
  REGION 1..107: note = Description of Artificial Sequence: Synthetic polypeptide
EIMMTQSPAILSVSPGDRATLSCRASQSVKNNLAWYQKRPGQAPRLLIFDTSSRASGIPARFSGGGSGTEFTLTVNSMQSEDFATYYCQQYEEWPRTFGQGTKVEIK

SEQ ID NO: 427 - protein, 135 aa, synthetic construct
  REGION 1..135: note = Description of Artificial Sequence: Synthetic polypeptide
QPQLQESGPGLVEASETLSLTCTVSGDSTAACDYFWGWVRQPPGKGLEWIGSLSHCAGYYNSGWTYHNPSLKSRLTISLDTPKNQVFLKLNSVTAADTAIYYCARFGGDVLVYHDWPKPAWVDLWGRGVLVTVSS

SEQ ID NO: 428 - protein, 105 aa, synthetic construct
  REGION 1..105: note = Description of Artificial Sequence: Synthetic polypeptide
QSALTQPPSASGSPGQSITISCTGNINNFVSWYQQHPGKAPKLVIYGVNKRPSGVPDRFSGSKSGNAASLTVSGLQTDDEAVYYCGSLAGNWDVVFGGGTKLTVL

SEQ ID NO: 429 - protein, 135 aa, synthetic construct
  REGION 1..135: note = Description of Artificial Sequence: Synthetic polypeptide
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSS

SEQ ID NO: 430 - protein, 105 aa, synthetic construct
  REGION 1..105: note = Description of Artificial Sequence: Synthetic polypeptide
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVL

SEQ ID NO: 431 - protein, 135 aa, synthetic construct
  REGION 1..135: note = Description of Artificial Sequence: Synthetic polypeptide
QPQLQESGPGLVEASETLSLTCTVSGDSTAGCDYFWGWVRQPPGKGLEWIGGLSHCAGYYNTGWTYHNPSLKSRLTISLDTPKNQVFLKLNSVTAADTAIYYCARFDGEVLVYNDWPKPAWVDLWGRGTLVTVSS

SEQ ID NO: 432 - protein, 105 aa, synthetic construct
  REGION 1..105: note = Description of Artificial Sequence: Synthetic polypeptide
QSALTQPPSASGSPGQSITISCTGTSNNFVSWYQQHPAKAPKLVIYGVNKRPSGVPDRFSGSKSGNTASLTVSGLQTDDEAVYYCGSLVGNWDVIFGGGTKLTVL

SEQ ID NO: 433 - protein, 129 aa, synthetic construct
  REGION 1..129: note = Description of Artificial Sequence: Synthetic polypeptide
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSS

SEQ ID NO: 434 - protein, 110 aa, synthetic construct
  REGION 1..110: note = Description of Artificial Sequence: Synthetic polypeptide
QSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVL

SEQ ID NO: 435 - protein, 130 aa, synthetic construct
  REGION 1..130: note = Description of Artificial Sequence: Synthetic polypeptide
QVQLRESGPGLVKPSETLSLSCTVSQDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 436 - protein, 110 aa, synthetic construct
  REGION 1..110: note = Description of Artificial Sequence: Synthetic polypeptide
WASSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVL

SEQ ID NO: 437 - protein, 130 aa, synthetic construct
  REGION 1..130: note = Description of Artificial Sequence: Synthetic polypeptide
QVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 438 - protein, 108 aa, synthetic construct
  REGION 1..108: note = Description of Artificial Sequence: Synthetic polypeptide
SSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVL

SEQ ID NO: 439 - protein, 130 aa, synthetic construct
  REGION 1..130: note = Description of Artificial Sequence: Synthetic polypeptide
EVQLRESGPRLVKPSETLSLSCDVFGDSRPSDHSWTWVRQPPGKALEWIGDVHYNGDNTYNPSLRGRVKIDVDRSTHRFSLTLKSLTAADTGIYFCARNVIRVFGVISLGEWFHYGMDVWGPGTAVIVSS

SEQ ID NO: 440 - protein, 108 aa, synthetic construct
  REGION 1..108: note = Description of Artificial Sequence: Synthetic polypeptide
SSELTQAPSVSVSPGQTATIACSGPPLASRYTYWYRQKPGQAPVLIIFRDRQFPSGVSGRFSASKSGTTATLTIRDVQVEDEGDYYCQSSDTSDSYKMFGGGTTLTVL

SEQ ID NO: 441 - protein, 130 aa, synthetic construct
  REGION 1..130: note = Description of Artificial Sequence: Synthetic polypeptide
EVQLRESGPGLVKPSETLSLSCDVFGDSRPSDHSWTWVRQPPGKALEWIGDVHYNGDTTYNPSLRGRVKIDVDRSTHRFSLTLNSLTAADTGIYFCARNVIRVFGVISLGEWFHYGMDVWGQGTAVTVSS

SEQ ID NO: 442 - protein, 108 aa, synthetic construct; identical to SEQ ID NO: 440
  REGION 1..108: note = Description of Artificial Sequence: Synthetic polypeptide

SEQ ID NO: 443 - protein, 130 aa, synthetic construct
  REGION 1..130: note = Description of Artificial Sequence: Synthetic polypeptide
QVQLRESGPGLVKPSETLSLTCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 444 - protein, 108 aa, synthetic construct
  REGION 1..108: note = Description of Artificial Sequence: Synthetic polypeptide
SSELTQPPSVSVSPGQTAKITCSGAALTSRFTYWYRQKPGQAPVLIISRTSQRSSGWSGRFSASWSGTTVTLTIRGVQADDEGDYYCQSSDTSDSYKMFGGGTKLTVL

SEQ ID NO: 445 - protein, 130 aa, synthetic construct
  REGION 1..130: note = Description of Artificial Sequence: Synthetic polypeptide
EVQLRESGPGLVKPSGNMALTCTISGDSRPSDHSWTWVRQSPGKALEWIGDIHYGGDITYNPSLRSRVKLEVDTSTNRFFLKMTSLTVADTGIYFCARNVIRVFGVIALGEWFHYGMDVWGQGTAITVSP

SEQ ID NO: 446 - protein, 108 aa, synthetic construct
  REGION 1..108: note = Description of Artificial Sequence: Synthetic polypeptide
SSELTQTPSVTVSPGETARIACSGPPLASRYCYWYRQKPGQAPVLIIFRDRQFSSGMSGRFASSHSGTTVTLTIRDVRVEDEADYYCQSSDINDSYKMFGGGTKVTVL

SEQ ID NO: 447 - protein, 130 aa, synthetic construct
  REGION 1..130: note = Description of Artificial Sequence: Synthetic polypeptide
EVQLRESGPGLVKPSGNMALTCTISGDSRPSDHSWTWVRQSPGKTLEWIGDIHYGGDITYNPSLRSRVKLEVDTSSNRFFLKMTSLTVADTGIYFCARNVIRVFGVIALGEWFHYGMDVWGQGTAITVSP

SEQ ID NO: 448 - protein, 108 aa, synthetic construct
  REGION 1..108: note = Description of Artificial Sequence: Synthetic polypeptide
SSELTQTASVTVSPGETARIACSGPPLASRYCYWYRQKPGQAPVLIIFRDRQFSSGISGRFSSSQSGTTVTLTIRDVRVEDEADYYCQSSDTSDSFKMFGGGTKLTVL

SEQ ID NO: 449 - protein, 130 aa, synthetic construct
  REGION 1..130: note = Description of Artificial Sequence: Synthetic polypeptide
QVQLRESGPGLVKPSGNMALTCTISGDSRPSDHSWTWVRQSPGKALEWIGDIHYGGDITYNPSLRSRVELEVDRSTNRFFLKMTSLSVADTGMYFCARNVIRVFGVIALGEWFHYGMDVWGQGTAITVSP

SEQ ID NO: 450 - protein, 108 aa, synthetic construct
  REGION 1..108: note = Description of Artificial Sequence: Synthetic polypeptide
SSELTQAPSVTVSPGDTARIACSGPPLATRYCYWYRQKSGQAPVLIIFRDRQFSSGVSGRFSSSQSGSTVTLTIRDVRVEDEADYYCQSSDTSDSYKMFGGGTKLTVL

SEQ ID NO: 451 - protein, 128 aa, synthetic construct
  REGION 1..128: note = Description of Artificial Sequence: Synthetic polypeptide
QVQLRESGPGLVKPSETLSLSCDVFGDSRPSDHSWTWVRQPPGKALEWIGDIHYNGDKTYNPSLRGRVKIDVDRSTHRFSLTLNSLTAADTGMYFCARNVIRVFGVISLGEWFHYGMDVWGPGTAVTV

SEQ ID NO: 452 - protein, 108 aa, synthetic construct
  REGION 1..108: note = Description of Artificial Sequence: Synthetic polypeptide
SSELTQAPSVSVSPGQTARIACSGPPLASRYTYWYRQKPGQAPVLIIFRDRQFPSGVSGRFSASKSGTTGTLTIRDVQAEDEGDYYCQSSDTSDSYKMFGGGTTLTVL

SEQ ID NO: 453 - protein, 130 aa, synthetic construct
  REGION 1..130: note = Description of Artificial Sequence: Synthetic polypeptide
QVQLRESGPGLVKPSGNMALTCTISGDSRPSDHSWTWVRQSPGKALEWIGDIHYGGDITYNPSLRSRVKLEVDTSSNRFFLKMTSLTVADTGIYFCARNVIRVFGVIALGEWFHYGMDVWGQGTAITVSP

SEQ ID NO: 454 - protein, 108 aa, synthetic construct
  REGION 1..108: note = Description of Artificial Sequence: Synthetic polypeptide
SSELTQAPSVTLSPGETARIACSGPPLASRYCYWYRQKPGQAPVLIIFRDRQFSSGISGRFSSSQSGTTVTLTIRDVRVEDEADYYCQSSDNSDSFKMFGGGTKLTVL

SEQ ID NO: 455 - protein, 133 aa, synthetic construct
  REGION 1..133: note = Description of Artificial Sequence: Synthetic polypeptide
AEQLVESGGGLVPPGRSLRLSCSASGFYFPDYAMAWVRQAPGQGLQWVGFMRGWAYGGSAQFAAFAVGKFAISRDDGRNVVYLDVKNPTFEDTGVYFCAREQRNKDYRYGQEGFGYSYGMDVWGRGTTVVVST

SEQ ID NO: 456 - protein, 107 aa, synthetic construct
  REGION 1..107: note = Description of Artificial Sequence: Synthetic polypeptide
DIHMTQSPVSLSASVGDRVTITCRASHFIANYVNWYQQKPGKAPTLLIFESSTLQRGVPSRFSAYGDGTEFTLSINTLQPEDFASYICQQSHSPPVTFGAGTRVDQK

SEQ ID NO: 457 - protein, 133 aa, synthetic construct
  REGION 1..133: note = Description of Artificial Sequence: Synthetic polypeptide
EERLVESGGGLVPPGRSLRLSCSAFDFYFPDYAMAWVRQAPGKGLEWIGFIRGWAYGQAAQYGKSASGRMTISRDDSRRVVYLDIKSPIEEDTGAYFCAREQRGGDGRYSGDGFGYPYGMDVWGRGTMVTVSA

SEQ ID NO: 458 - protein, 107 aa, synthetic construct
  REGION 1..107: note = Description of Artificial Sequence: Synthetic polypeptide
DILMTQSPVSLSASIGERITITCRASHFIANYVNWYQQRPGKAPKLLIFQSWTLNRGIPSRFSGYGDGTEFTLSISALQSEDFGTYICQQSHSPPLSFGGGTRVDQT

SEQ ID NO: 459 - protein, 133 aa, synthetic construct
  REGION 1..133: note = Description of Artificial Sequence: Synthetic polypeptide
EERLVESGGGLVPPGRSLRLSCSAFDFYFPDYAMAWVRQAPGRALEWIGFIRGWAYGQSAQYGKSASGRMTISRDDSRRVVYLDIKSPTHEDTGVYFCAREQRGANGRYGGDGFGYSYGMDVWGRGTMVSVSA

SEQ ID NO: 460 - protein, 107 aa, synthetic construct
  REGION 1..107: note = Description of Artificial Sequence: Synthetic polypeptide
DIQMTQSPFTLSASVGERVTITCRASHFIANYVNWYQQRPGRAPKLLIFESSTLNRGVPSRFSGSGDGTEFTLSISALQSEDFATYICQQSHSPPVSFGGGTRVDQT

SEQ ID NO: 461 - protein, 26 aa, synthetic construct
  REGION 1..26: note = Description of Artificial Sequence: Synthetic peptide
ARARRGQRIYGVVSFGEFFYYYSMDV

SEQ ID NO: 462 - protein, 132 aa, synthetic construct; identical to SEQ ID NO: 402
  REGION 1..132: note = Description of Artificial Sequence: Synthetic polypeptide

SEQ ID NO: 463 - protein, 105 aa, synthetic construct; identical to SEQ ID NO: 404
  REGION 1..105: note = Description of Artificial Sequence: Synthetic polypeptide

SEQ ID NO: 464 - protein, 132 aa, synthetic construct
  REGION 1..132: note = Description of Artificial Sequence: Synthetic polypeptide
QMQLQESGPGLVKPSETLSLTCSVSGASISDSYWSWIRQPPGKGLEWIGYVHKSGDTNYSPSLKSRVNLSLDTSKNQVSLSLSAATAADSGVYYCARTLHGRRIYGIVAFNEWFTYFYMDVWGNGTQVTVSS

SEQ ID NO: 465 - protein, 105 aa, synthetic construct
  REGION 1..105: note = Description of Artificial Sequence: Synthetic polypeptide
SDISVAPGETARISCGEKSLGSRAVQWYQQRAGQAPSLIIYNNQDRPSGIPERFSGSPDSGFGTTATLTITSVEAGDEADYYCHIWDSRVPTKWVFGGGTTLTVL

SEQ ID NO: 466 - protein, 132 aa, synthetic construct
  REGION 1..132: note = Description of Artificial Sequence: Synthetic polypeptide
QVQLQESGPGLVKPSETLSVTCSVSGDSMNNYYWTWIRQSPGKGLEWIGYISDRESATYNPSLNSRVTISRDTSKNQFSLKLNSVTPADTAVYYCARARRGQRIYGVVSFGEFFYYYSMDVWGKGTTVTVSS

SEQ ID NO: 467 - protein, 108 aa, synthetic construct
  REGION 1..108: note = Description of Artificial Sequence: Synthetic polypeptide
SPVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 468 - protein, 4 aa, Human immunodeficiency virus 1
KLIC